(19) United States
(12) Patent Application Publication
(10) Pub. No.: US 2025/0368741 A1
(43) Pub. Date: Dec. 4, 2025

(54) FCRN ANTAGONIST MOLECULES AND METHODS OF USE THEREOF

(71) Applicant: argenx BV, Ghent (BE)

(72) Inventors: Hilde Stals, Ghent (BE); Kris Meerschaert, Ghent (BE); Valérie Hanssens, Ghent (BE); Filip Borgions, Ghent (BE)

(21) Appl. No.: 19/207,833

(22) Filed: May 14, 2025

Related U.S. Application Data
(63) Continuation of application No. PCT/IB2023/000696, filed on Nov. 14, 2023.
(60) Provisional application No. 63/383,599, filed on Nov. 14, 2022.

(51) Int. Cl. C07K 16/28; A61K 9/08; A61K 47/02; A61K 47/18; A61K 47/26

(52) U.S. Cl. CPC C07K 16/283; A61K 9/08; A61K 47/02; A61K 47/183; A61K 47/26; C07K 2317/52; C07K 2317/72

(57) ABSTRACT

The disclosure provides populations of FcRn antagonist molecules, mixtures of these populations and methods of using these populations to reduce the level of serum IgG autoantibodies in a subject.

### RELATED APPLICATIONS

This application is a Continuation of International Patent Application No. PCT/IB2023/000696, filed Nov. 14, 2023, which claims priority to U.S. Provisional Patent Application Ser. No. 63/383,599, filed Nov. 14, 2022, the entire disclosure of each of which is hereby incorporated herein by reference.

### REFERENCE TO SEQUENCE LISTING

This application contains a sequence listing which has been submitted electronically in ST.26 format and is hereby incorporated by reference in its entirety (said ST.26 copy, created on Nov. 7, 2023, is named “404373_T2213WO.xml” and is 39,782 bytes in size).

### FIELD

The present disclosure relates to FcRn antagonist molecules, compositions comprising these FcRn antagonist molecules, and methods of reducing the level of serum IgG antibodies (e.g., autoantibodies) in a subject using these FcRn antagonist molecules and compositions.

### BACKGROUND

It is estimated that more than 2.5% of the human population is affected by autoantibody-driven autoimmune diseases, in which autoreactive antibodies are directly pathogenic. The half-life of IgG in the serum is prolonged relative to the serum half-life of other plasma proteins (Roopenian et al., J. Immunology 170:3528 (2003); Junghans and Anderson, Proc. Natl. Acad. Sci. USA 93:5512 (1996)). This long half-life is due, in part, to the binding of the Fc region of IgG to the Fc receptor, FcRn. Although FcRn was originally characterized as a neonatal transport receptor for maternal IgG, it also functions in adults to protect IgG from degradation. FcRn binds to pinocytosed IgG and protects the IgG from transport to degradative lysosomes by recycling it back to the extracellular compartment. This recycling is facilitated by the pH dependent binding of IgG to FcRn, where the IgG/FcRn interaction is stronger at acidic endosomal pH than at extracellular physiological pH.

When the serum concentration of IgG reaches a level that exceeds available FcRn molecules, unbound IgG is not protected from degradative mechanisms and will consequently have a reduced serum half-life. Thus, inhibition of IgG binding to FcRn reduces the serum half-life of IgG by preventing IgG endosomal recycling of IgG. Accordingly, agents that antagonize the binding of IgG to FcRn may be useful for regulating, treating or preventing antibody-mediated disorders, such as autoimmune diseases, inflammatory diseases, etc.

There is a need in the art for agents that antagonize FcRn binding to IgG for use in the treatment of antibody-mediated disorders.

### SUMMARY

The present disclosure is directed to novel FcRn antagonist molecules, compositions comprising these FcRn antagonist molecules, and methods of reducing the level of serum IgG antibodies (e.g., autoantibodies) in a subject using these FcRn antagonist molecules and compositions. Nucleic acids encoding the FcRn antagonist molecules as well as vectors, host cells, methods of manufacture, and methods for their use in treating IgG antibody-mediated disorders are also provided herein. The FcRn antagonist molecules provided herein are particularly advantageous in that they are all capable of rapidly reducing the level of serum IgG antibodies in a subject and exhibit long term stability in aqueous formulations.

The present disclosure provides a composition comprising a population of FcRn antagonist molecules, wherein at least a portion of the FcRn antagonist molecules in the population consist of a variant Fc region comprising a dimer of a first Fc domain and a second Fc domain, wherein the amino acid sequence of the first and second Fc domain consists of SEQ ID NO: 1, provided that the population is not a homogeneous population of homodimeric FcRn antagonist molecules in which the amino acid sequence of both the first and the second Fc domain consists of SEQ ID NO: 2, 3, 20, or 21. In some embodiments, each FcRn antagonist molecule in the population consists of a variant Fc region comprising a dimer of a first Fc domain and a second Fc domain, wherein the amino acid sequence of the first and second Fc domain consists of SEQ ID NO: 1.

In some embodiments, the amino acid sequences of the first and the second Fc domains consist of SEQ ID NO: 3 and SEQ ID NO: 12, respectively. In some embodiments, the amino acid sequences of the first and the second Fc domains consist of SEQ ID NO: 3 and SEQ ID NO: 9, respectively. In some embodiments, the amino acid sequences of the first and the second Fc domains consist of SEQ ID NO: 2 and SEQ ID NO: 3, respectively. In some embodiments, the amino acid sequences of the first and the second Fc domains consist of SEQ ID NO: 3 and SEQ ID NO: 6, respectively.

In some embodiments, the amino acid sequence of the first Fc domain consists of any one of SEQ ID NOs: 2-22, and the amino acid sequence of the second Fc domain consists of any one of SEQ ID NOs: 2-22. In some embodiments, the amino acid sequence of both the first and the second Fc domain consists of SEQ ID NO: 5. In some embodiments, the amino acid sequence of both the first and the second Fc domain consists of SEQ ID NO: 6. In some embodiments, the amino acid sequence of both the first and the second Fc domain consists of SEQ ID NO: 7. In some embodiments, the amino acid sequence of both the first and the second Fc domain consists of SEQ ID NO: 8. In some embodiments, the amino acid sequence of both the first and the second Fc domain consists of SEQ ID NO: 9. In some embodiments, the amino acid sequence of both the first and the second Fc domain consists of SEQ ID NO: 10. In some embodiments, the amino acid sequence of both the first and the second Fc domain consists of SEQ ID NO: 11. In some embodiments, the amino acid sequence of both the first and the second Fc domain consists of SEQ ID NO: 12. In some embodiments, the amino acid sequence of both the first and the second Fc domain consists of SEQ ID NO: 13. In some embodiments, the amino acid sequence of both the first and the second Fc domain consists of SEQ ID NO: 14. In some embodiments, the amino acid sequence of both the first and the second Fc domain consists of SEQ ID NO: 15. In some embodiments, the amino acid sequence of both the first and the second Fc domain consists of SEQ ID NO: 16. In some embodiments, the amino acid sequence of both the first and the second Fc domain consists of SEQ ID NO: 17. In some embodiments, the amino acid sequence of both the first and the second Fc domain consists of SEQ ID NO: 18. In some embodiments, the amino acid sequence of both the first and the second Fc domain consists of SEQ ID NO: 19. In some embodiments, the amino acid sequence of both the first and the second Fc domain consists of SEQ ID NO: 22.

In some embodiments, the population comprises: a first subpopulation of FcRn antagonist molecules, wherein the amino acid sequences of both the first and the second Fc domains of the FcRn antagonist molecules in the first subpopulation consist of SEQ ID NO: 3; and at least one of: a second subpopulation of FcRn antagonist molecules, wherein the amino acid sequences of the first and the second Fc domains of the FcRn antagonist molecules in the second subpopulation consist of SEQ ID NO: 3 and SEQ ID NO: 12, respectively; a third subpopulation of FcRn antagonist molecules, wherein the amino acid sequences of the first and the second Fc domains of the FcRn antagonist molecules in the third subpopulation consist of SEQ ID NO: 3 and SEQ ID NO: 9, respectively; a fourth subpopulation of FcRn antagonist molecules, wherein the amino acid sequences of both the first and the second Fc domains of the FcRn antagonist molecules in the fourth subpopulation consist of SEQ ID NO: 3, and wherein two asparagine residues in each FcRn antagonist molecule in the fourth subpopulation are deaminated; a fifth subpopulation of FcRn antagonist molecules, wherein the amino acid sequences of the first and the second Fc domains of the FcRn antagonist molecules in the fifth subpopulation consist of SEQ ID NO: 3 and SEQ ID NO: 9, respectively, and wherein one asparagine residue in each FcRn antagonist molecule in the fifth subpopulation is deaminated; a sixth subpopulation of FcRn antagonist molecules, wherein the amino acid sequences of the first and the second Fc domains of the FcRn antagonist molecules in the sixth subpopulation consist of SEQ ID NO: 2 and SEQ ID NO: 3, respectively; a seventh subpopulation of FcRn antagonist molecules, wherein the amino acid sequences of the first and the second Fc domains of the FcRn antagonist molecules in the seventh subpopulation consist of SEQ ID NO: 2 and SEQ ID NO: 3, respectively, and wherein one methionine residue or one tryptophan residue in each FcRn antagonist molecule in the seventh subpopulation is oxidized; an eighth subpopulation of FcRn antagonist molecules, wherein the amino acid sequences of both the first and the second Fc domains of the FcRn antagonist molecules in the eighth subpopulation consist of SEQ ID NO: 2; a ninth subpopulation of FcRn antagonist molecules, wherein the amino acid sequences of the first and the second Fc domains of the FcRn antagonist molecules in the ninth subpopulation consist of SEQ ID NO: 3 and SEQ ID NO: 6, respectively; a tenth subpopulation of FcRn antagonist molecules, wherein the amino acid sequences of the first and the second Fc domains of the FcRn antagonist molecules in the tenth subpopulation consist of SEQ ID NO: 2 and SEQ ID NO: 3, respectively, and wherein one methionine residue or one tryptophan residue in each FcRn antagonist molecule in the tenth subpopulation is oxidized; and an eleventh subpopulation of FcRn antagonist molecules, wherein the amino acid sequences of both the first and the second Fc domains of the FcRn antagonist molecules in the eleventh subpopulation consist of SEQ ID NO: 3, and wherein two amino acid residues, independently selected from a methionine residue and a tryptophan, in each FcRn antagonist molecule in the eleventh subpopulation are oxidized.

In some embodiments, the population comprises 2, 3, 4, 5, 6, 7, 8, 9, 10, or all of the subpopulations set forth above. In some embodiments, the population comprises the seventh, ninth, or eleventh subpopulations. In some embodiments, the population comprises the seventh, ninth, and eleventh subpopulations.

In some embodiments, the first subpopulation is at least 55% of the population, optionally the first subpopulation is 60% to 70% of the population. In some embodiments, the second subpopulation is no more than 2.5% of the population, optionally the second subpopulation is 1% to 2.5% of the population. In some embodiments, the third subpopulation is no more than 2.5% of the population, optionally the third subpopulation is 1% to 2.5% of the population. In some embodiments, the fourth subpopulation is no more than 5% of the population, optionally the fourth subpopulation is 2% to 5% of the population. In some embodiments, the fifth subpopulation is no more than 10% of the population, optionally the fifth subpopulation is 7% to 10% of the population. In some embodiments, the sixth subpopulation is no more than 20% of the population, optionally the sixth subpopulation is 7% to 14% of the population. In some embodiments, the seventh subpopulation is no more than 6% of the population, optionally the seventh subpopulation is 1.5% to 2.5% of the population. In some embodiments, the eighth subpopulation is no more than 8% of the population, optionally the eighth subpopulation is 3.5% to 7.5% of the population. In some embodiments, the ninth subpopulation is no more than 3.5% of the population, optionally the ninth subpopulation is 0.5% to 3.5% of the population. In some embodiments, the tenth subpopulation is no more than 1% of the population. In some embodiments, the eleventh subpopulation is no more than 1% of the population.

In some embodiments, at least 97%, optionally 97% to 99%, of the Fc domains in the population comprise an N-glycan at EU position 297. In some embodiments, at least 50%, optionally 50% to 70%, of the Fc domains in the population comprise a G0F N-glycan at EU position 297. In some embodiments, at least 20%, optionally 20% to 30%, of the Fc domains in the population comprise a G1F N-glycan at EU position 297. In some embodiments, at least 5%, optionally 8% to 10%, of the Fc domains in the population comprise a G2F N-glycan at EU position 297. In some embodiments, at least 2%, optionally 2% to 5%, of the Fc domains in the population comprise a G0 N-glycan at EU position 297.

In some embodiments, at least 40%, optionally 40% to 55%, of the population comprise a first Fc domain comprising a G0F N-glycan at EU position 297 and a second Fc domain comprising a G0F N-glycan at EU position 297. In some embodiments, at least 20%, optionally 20% to 25%, of the population comprise a first Fc domain comprising a G0F N-glycan at EU position 297 and a second Fc domain comprising a G1F N-glycan at EU position 297. In some embodiments, at least 10%, optionally 10% to 15%, of the population comprise either a first Fc domain comprising a G1F N-glycan at EU position 297 and a second Fc domain comprising a GIF N-glycan at EU position 297, or a first Fc domain comprising G0F N-glycan at EU position 297 and a second Fc domain comprising a G2F N-glycan at EU position 297. In some embodiments, at least 5%, optionally 5% to 10%, of the population comprise a first Fc domain comprising a GIF N-glycan at EU position 297 and a second Fc domain comprising a G2F N-glycan at EU position 297. In some embodiments, at least 2%, optionally 2% to 4%, of the population comprise a first Fc domain comprising a G2F N-glycan at EU position 297 and a second Fc domain comprising a G2F N-glycan at EU position 297. In some embodiments, at least 4%, optionally 4% to 6%, of the population comprise a first Fc domain comprising a G0F N-glycan at EU position 297 and a second Fc domain comprising a G0 N-glycan at EU position 297.

The disclosure also provides a composition comprising an FcRn antagonist molecule consisting of a variant Fc region comprising a dimer of a first Fc domain and a second Fc domain, wherein the amino acid sequence of the first and second Fc domain consists of SEQ ID NO: 1, and wherein at least one Fc domain comprises a G0F N-glycan at EU position 297, a G1F N-glycan at EU position 297, a G2F N-glycan at EU position 297, or a G0 N-glycan at EU position 297.

In some embodiments, the first Fc domain comprises a G0F N-glycan at EU position 297, and the second Fc domain comprises a G0F N-glycan at EU position 297. In some embodiments, the first Fc domain comprises a G0F N-glycan at EU position 297, and the second Fc domain comprises a G1F N-glycan at EU position 297. In some embodiments, the first Fc domain comprises a G0F N-glycan at EU position 297, and the second Fc domain comprises a G2F N-glycan at EU position 297. In some embodiments, the first Fc domain comprises a G1F N-glycan at EU position 297, and the second Fc domain comprises a G1F N-glycan at EU position 297. In some embodiments, the first Fc domain comprises a G2F N-glycan at EU position 297, and the second Fc domain comprises a G2F N-glycan at EU position 297. In some embodiments, the first Fc domain comprises a G0F N-glycan at EU position 297, and the second Fc domain comprises a G0 N-glycan at EU position 297. In some embodiments, the first Fc domain comprises a G0 N-glycan at EU position 297, and the second Fc domain comprises a G0 N-glycan at EU position 297. In some embodiments, the first Fc domain comprises a G1F N-glycan at EU position 297, and the second Fc domain comprises a G2F+NANA N-glycan at EU position 297. In some embodiments, the first Fc domain comprises a G2F N-glycan at EU position 297, and the second Fc domain comprises a G2F+2×NANA N-glycan at EU position 297.

In some embodiments, the amino acid sequences of the first and the second Fc domains consist of SEQ ID NO: 3 and SEQ ID NO: 12, respectively. In some embodiments, the amino acid sequences of the first and the second Fc domains consist of SEQ ID NO: 3 and SEQ ID NO: 9, respectively. In some embodiments, the amino acid sequences of the first and the second Fc domains consist of SEQ ID NO: 2 and SEQ ID NO: 3, respectively. In some embodiments, the amino acid sequences of the first and the second Fc domains consist of SEQ ID NO: 3 and SEQ ID NO: 6, respectively.

In some embodiments, the amino acid sequence of the first Fc domain consists of any one of SEQ ID NOs: 2-22, and the amino acid sequence of the second Fc domain consists of any one of SEQ ID NOs: 2-22. In some embodiments, the amino acid sequence of both the first and the second Fc domain consists of SEQ ID NO: 2. In some embodiments, the amino acid sequence of both the first and the second Fc domain consists of SEQ ID NO: 3. In some embodiments, the amino acid sequence of both the first and the second Fc domain consists of SEQ ID NO: 4. In some embodiments, the amino acid sequence of both the first and the second Fc domain consists of SEQ ID NO: 5. In some embodiments, the amino acid sequence of both the first and the second Fc domain consists of SEQ ID NO: 6. In some embodiments, the amino acid sequence of both the first and the second Fc domain consists of SEQ ID NO: 7. In some embodiments, the amino acid sequence of both the first and the second Fc domain consists of SEQ ID NO: 8. In some embodiments, the amino acid sequence of both the first and the second Fc domain consists of SEQ ID NO: 9. In some embodiments, the amino acid sequence of both the first and the second Fc domain consists of SEQ ID NO: 10. In some embodiments, the amino acid sequence of both the first and the second Fc domain consists of SEQ ID NO: 11. In some embodiments, the amino acid sequence of both the first and the second Fc domain consists of SEQ ID NO: 12. In some embodiments, the amino acid sequence of both the first and the second Fc domain consists of SEQ ID NO: 13. In some embodiments, the amino acid sequence of both the first and the second Fc domain consists of SEQ ID NO: 14. In some embodiments, the amino acid sequence of both the first and the second Fc domain consists of SEQ ID NO: 15. In some embodiments, the amino acid sequence of both the first and the second Fc domain consists of SEQ ID NO: 16. In some embodiments, the amino acid sequence of both the first and the second Fc domain consists of SEQ ID NO: 17. In some embodiments, the amino acid sequence of both the first and the second Fc domain consists of SEQ ID NO: 18. In some embodiments, the amino acid sequence of both the first and the second Fc domain consists of SEQ ID NO: 19. In some embodiments, the amino acid sequence of both the first and the second Fc domain consists of SEQ ID NO: 20. In some embodiments, the amino acid sequence of both the first and the second Fc domain consists of SEQ ID NO: 21. In some embodiments, the amino acid sequence of both the first and the second Fc domain consists of SEQ ID NO: 22.

In some embodiments, at least 85%, optionally 85% to 95%, of the Fc domains in the population lack an amino acid at EU position 441. In some embodiments, no more than 15%, optionally 5% to 15%, of the Fc domains in the population have glycine and lysine at EU positions 440 and 441, respectively. In some embodiments, no more than 1% of the Fc domains in the population lack amino acids at EU positions 440 and 441 and comprise amidated proline at EU position 439.

In some embodiments, at least 95%, optionally 95% to 99%, of the Fc domains in the population have aspartate, lysine, threonine, histidine, threonine, and cysteine, at EU positions 221, 222, 223, 224, 225, and 226, respectively. In some embodiments, no more than 1% of the Fc domains in the population lack an amino acid at EU position 221, and have lysine, threonine, histidine, threonine, and cysteine at EU positions 222, 223, 224, 225, and 226, respectively. In some embodiments, no more than 1% of the Fc domains in the population lack amino acids at EU positions 221 and 222, and have threonine, histidine, threonine, and cysteine at EU positions 223, 224, 225, and 226, respectively. In some embodiments, no more than 2% of the Fc domains in the population lack amino acids at EU positions 221-224, and have threonine, and cysteine at EU positions 225 and 226, respectively. In some embodiments, no more than 1% of the Fc domains in the population lack amino acids at EU positions 221, 222, 223, 224, 225, and 226.

In some embodiments, no more than 1% of the Fc domains in the population have isomerization of the aspartate at EU position 280 or 401. In some embodiments, no more than 10% of the Fc domains in the population have deamidation of the asparagine at EU position 384, 389, or 390. In some embodiments, no more than 3% of the Fc domains in the population have deamidation of the asparagine at EU position 315. In some embodiments, no more than 3% of the Fc domains in the population have deamidation of the asparagine at EU position 361. In some embodiments, no more than 1% of the Fc domains in the population have deamidation of the asparagine at EU position 276 or 286. In some embodiments, no more than 5% of the Fc domains have oxidization of the methionine at EU position 428. In some embodiments, no more than 1% of the Fc domains have amidation of the proline at EU position 445. In some embodiments, no more than 1% of the Fc domains have oxidization of the tryptophan at EU position 277.

In some embodiments, no more than 0.5% of the FcRn antagonist molecules in the population are aggregated. In some embodiments, at least 95%, optionally at least 99%, of the dimers in the population are linked by at least one disulfide bond. In some embodiments, the average molecular weight of non-aggregated FcRn antagonist molecules in the population is 54 to 56 kDa, optionally 54.4 to 54.7 kDa. In some embodiments, the percentage of free thiol groups in the population is no more than 1%.

In some embodiments, at least 35%, optionally 35% to 55%, of the Fc domains in the population comprise galactose. In some embodiments, at least 90%, optionally 90% to 98%, of the Fc domains in the population comprise fucose. In some embodiments, at most 1.5%, optionally 0.5% to 1.5%, of the Fc domains in the population comprise sialic acid.

In some embodiments, the composition described above or herein comprises an aqueous solution comprising about 25 mM sodium phosphate, about 100 mM sodium chloride, and about 150 mM L-arginine, and about 0.02% (w/v) polysorbate 80, wherein the composition has a pH of about 6.7. In some embodiments, this composition comprises 20 mg/ml of the population of FcRn antagonist molecules.

In some embodiments, the composition described above or herein comprises an aqueous solution comprising about 4 mM sodium phosphate, about 146 mM sodium chloride, and about 24 mM L-arginine, and about 0.0032% (w/v) polysorbate 80, wherein the composition has a pH of about 6.7. In some embodiments, this composition comprises about 3.2 mg/ml of the population of FcRn antagonist molecules.

In some embodiments, the composition described above or herein comprises an aqueous solution comprising about 20 mM L-histidine, about 100 mM sodium chloride, about 60 mM sucrose, about 10 mM L-methionine, and about 0.04% (w/v) polysorbate 20, wherein the composition has a pH of about 6.0. In some embodiments, this composition comprises about 180 mg/ml of the population of FcRn antagonist molecules.

In some embodiments, the composition described above or herein comprises an aqueous solution comprising about 20 mM L-histidine, about 50 mM L-arginine, about 100 mM sodium chloride, about 60 mM sucrose, about 10 mM L-methionine, and about 0.04% (w/v) polysorbate 80, wherein the composition has a pH of about 6.0. In some embodiments, this composition comprises about 200 mg/ml of the population of FcRn antagonist molecules.

The present disclosure also provides an FcRn antagonist molecule consisting of a variant Fc region comprising a homodimer of a first Fc domain and a second Fc domain, wherein the amino acid sequence of both the first and the second Fc domain consists of any one of SEQ ID NOs: 5-20 and 22. The present disclosure also provides a polynucleotide encoding the FcRn antagonist molecule. The present disclosure also provides a vector comprising the polynucleotide.

The present disclosure also provides a cell comprising the polynucleotide. The present disclosure also provides a method of making an FcRn antagonist molecule, the method comprising culturing the cell described above and herein under conditions such that the polynucleotide described above and herein is expressed and the FcRn antagonist molecule is produced. In some embodiments, the method also includes isolating the FcRn antagonist molecule from the cell.

The present disclosure also provides a method comprising mixing the compositions provided above and herein, the FcRn antagonists provided above and herein, the polynucleotides provided above and herein, the vectors provided above and herein, or the cells provided above and herein with one or more pharmaceutically acceptable excipients.

The present disclosure also provides a method of reducing the level of serum IgG autoantibodies in a subject, the method comprising administering to the subject any one or more of the compositions provided above and herein, any one or more of the FcRn antagonists provided above and herein, any one or more of the polynucleotides provided above and herein, any one or more of the vectors provided above and herein, or any one or more of the cells provided above and herein.

The present disclosure also provides a method of treating an autoimmune disease in a subject, the method comprising administering to the subject any one or more of the compositions provided above and herein, any one or more of the FcRn antagonists provided above and herein, any one or more of the polynucleotides provided above and herein, any one or more of the vectors provided above and herein, or any one or more of the cells provided above and herein.

The present disclosure also provides any one or more of the compositions provided above and herein, any one or more of the FcRn antagonists provided above and herein, any one or more of the polynucleotides provided above and herein, any one or more of the vectors provided above and herein, or any one or more of the cells provided above and herein, for use in the treatment of an autoimmune disease.

The present disclosure also provides a use of any one or more of the compositions provided above and herein, any one or more of the FcRn antagonists provided above and herein, any one or more of the polynucleotides provided above and herein, any one or more of the vectors provided above and herein, or any one or more of the cells provided above and herein, for the treatment of an autoimmune disease.

The present disclosure also provides any one or more of the composition provided above and herein, any one or more of the FcRn antagonists provided above and herein, any one or more of the polynucleotides provided above and herein, any one or more of the vectors provided above and herein, or any one or more of the cells provided above and herein, for use in the manufacture of a medicament for the treatment of an autoimmune disease.

The present disclosure also provides any one or more of the compositions provided above and herein, any one or more of the FcRn antagonists provided above and herein, any one or more of the polynucleotides provided above and herein, any one or more of the vectors provided above and herein, or any one or more of the cells provided above and herein, for use in medicine.

### BRIEF DESCRIPTION OF THE DRAWINGS

FIG. 1 depicts chromatograms of the cation exchange high performance liquid chromatography (CEX HPLC) fractionation of FcRn antagonist reference standards Batch 2 and Batch 3. In each case, absorbance at 220 nm (AU) is plotted against relative column retention time.

FIGS. 2A, 2B, and 2C depict a chromatogram of the CEX HPLC fractionation of FcRn antagonist for population of FcRn antagonist molecules manufactured from ARFCB11 cell line. In each case, absorbance at 220 nm (AU) is plotted against relative column retention time.

FIG. 3 depicts chromatograms of imaged capillary isoelectric focusing (icIEF) results for reference standards Batch 2 and Batch 3.

FIG. 4 depicts line graphs showing % area under the curve for charged variant 4 over storage time in months at −70° C., +5° C. and +25° C.

FIG. 5 depicts line graphs showing % area under the curve for charged variant 5 over storage time in months at −70° C., +5° C. and +25° C.

FIG. 6 depicts line graphs showing % area under the curve for charged variant 9 over storage time in months at −70° C., +5° C. and +25° C.

FIG. 7 depicts line graphs showing % area under the curve for charged variant 11 over storage time in months at −70° C., +5° C. and +25° C.

FIG. 8 depicts line graphs showing % area under the curve for charged isoform 1 over storage time in months at −70° C., +5° C. and +25° C.

FIG. 9 depicts line graphs showing % area under the curve for charged isoform 2 over storage time in months at −70° C., +5° C. and +25° C.

FIG. 10 depicts a hydrophilic interaction liquid chromatography (HILIC) chromatogram of 2-aminobenzamide (2-AB) labeled reference samples.

FIG. 11 depicts an enlarged version of a portion of FIG. 10.

FIG. 12 depicts a bar graph showing relative percent intensity of 2-AB labeled glycans in reference samples.

FIG. 13 depicts a bar graph showing relative percent intensity of galactosylation, fucosylation and sialyation in reference samples.

FIG. 14 depicts a set of absorbance plots of reversed-phase liquid chromatography with UV detection (RPLC-UV) 214 nm spectra showing electrospray ionization mass spectrometry (ESI-MS) results for reference standards Batch 1, Batch 2, and Batch 3.

FIG. 15 depicts deconvoluted spectra of the main peak observed in the RPLC-UV 214 nm profiles of reference samples with annotation of the glycoforms for samples Batch 1, Batch 2 and Batch 3.

FIG. 16 depicts a bar graph showing graphical representation of the N-glycosylation for samples Batch 1, Batch 2 and Batch 3.

FIG. 17 depicts the gel permeation high performance liquid chromatography (GP-HPLC) profile for reference standards Batch 2 and Batch 3.

FIG. 18 depicts a non-reducing capillary electrophoresis sodium dodecyl sulfate (CE-SDS) profile for reference standards Batch 2 and Batch 3.

### DETAILED DESCRIPTION

The present disclosure provides FcRn antagonist molecules and compositions comprising these FcRn antagonist molecules. The FcRn antagonist molecules and compositions provided herein are capable of reducing the serum level of IgG antibodies (e.g., IgG autoantibodies) in a subject. Nucleic acids encoding the FcRn antagonist molecules as well as vectors, host cells, methods of manufacture, and methods for their use in treating IgG antibody-mediated disorders are also provided herein.

### Definitions

As used herein, the term “FcRn” refers to a neonatal Fc receptor. Exemplary FcRn molecules include human FcRn encoded by the FCGRT gene as set forth in RefSeq NM 004107. The amino acid sequence of the corresponding protein is set forth in RefSeq NP_004098.

As used herein, the term “FcRn antagonist molecule” refers to any agent that specifically binds to FcRn and inhibits the binding of immunoglobulin to FcRn (e.g., human FcRn). In an embodiment, the FcRn antagonist comprises an Fc region (e.g., a variant Fc region disclosed herein) that specifically binds to FcRn and inhibits the binding of IgG to FcRn. In an embodiment, the FcRn antagonist is not a full-length IgG antibody. In an embodiment, the FcRn antagonist comprises an antigen-binding domain that binds a target antigen and a variant Fc region. In an embodiment, the term “FcRn antagonist molecule” refers to an antibody or antigen-binding fragment thereof that specifically binds to FcRn via its antigen binding domain and/or via its Fc region and inhibits the binding of the Fc region of immunoglobulin (e.g., IgG autoantibodies) to FcRn.

As used herein, the term “affinity” or “binding affinity” refers to the strength of the binding interaction between two molecules.

As used herein, the term “specifically binds” refers to the ability of any molecule to preferentially bind with a given target. For example, a molecule that specifically binds to a given target can bind to other molecules, generally with lower affinity as determined by, e.g., immunoassays, BIAcore™, KinExA 3000 instrument (Sapidyne Instruments, Boise, Id.), or other assays known in the art. In a specific embodiment, molecules that specifically bind to a given target bind to the antigen with a KD that is at least 2 logs, 2.5 logs, 3 logs, 4 logs or less than the KD when the molecules bind non-specifically to another target.

As used herein, the term “operably linked” refers to a linkage of polynucleotide sequence elements in a functional relationship. For example, a polynucleotide sequence is operably linked when it is placed into a functional relationship with another polynucleotide sequence. In some embodiments, a transcription regulatory polynucleotide sequence, e.g., a promoter, enhancer, or other expression control element is operably linked to a polynucleotide sequence that encodes a protein if it affects the transcription of the polynucleotide sequence that encodes the protein. Operably linked elements may be contiguous or non-contiguous.

As used herein, the term “linked” refers to a physical linkage (e.g., directly or indirectly linked) between amino acid sequences (e.g., different segments, regions, or domains). Linked regions, domains, and segments of the FcRn antagonist molecules of the disclosure may be contiguous or non-contiguous (e.g., linked to one another through a linker). In some embodiments, linkages are covalent. In some embodiments, linkages are non-covalent.

As used herein, the term “covalently linked” refers to the linkage of two molecules or chemical moieties by a covalent bond. In some embodiments, the covalent bond is a peptide bond or a disulfide bond. As used herein, the term “fused” refers to the linkage of two peptides by a peptide bond or a peptide linker. In some embodiments, two proteins are directly and contiguously fused together by a peptide bond. In some embodiments, two proteins are indirectly and non-contiguously fused through a peptide linker. In some embodiments, one protein is fused to a peptide linker by a peptide bond at a first position, and a second protein is fused to a peptide linker by a peptide bond at a second position. As used herein, the term “non-covalently linked” refers to the linkage of two molecules or chemical moieties by a non-covalent interaction or bond. In some embodiments, non-covalent interactions or bonds include hydrogen bonds, electrostatic bonds or interactions, halogen bonds, pi stacking, and van der Waals interactions.

As used herein, the terms “antibody” and “antibodies” include full-length antibodies, antigen-binding fragments of full-length antibodies, and molecules comprising antibody CDRs, VH regions, or VL regions. Examples of antibodies include monoclonal antibodies, recombinantly produced antibodies, monospecific antibodies, multi-specific antibodies (including bispecific antibodies), human antibodies, humanized antibodies, chimeric antibodies, immunoglobulins, synthetic antibodies, tetrameric antibodies comprising two heavy chain and two light chain molecules, an antibody light chain monomer, an antibody heavy chain monomer, an antibody light chain dimer, an antibody heavy chain dimer, an antibody light chain-antibody heavy chain pair, intrabodies, heteroconjugate antibodies, antibody-drug conjugates, single-domain antibodies (sdAb), monovalent antibodies, single chain antibodies or single-chain Fvs (scFv), camelid antibodies, single-domain antibodies (sdAb), humanized antibodies, affibody molecules, VHH fragments, Fab fragments, F(ab′)2 fragments, disulfide-linked Fvs (sdFv), anti-idiotypic (anti-Id) antibodies (including, e.g., anti-anti-Id antibodies), and antigen-binding fragments of any of the above. Antibodies can be of any isotype (e.g., IgG, IgE, IgM, IgD, IgA, or IgY), any subclass (e.g., IgG1, IgG2, IgG3, IgG4, IgA1, or IgA2), or species (e.g., mouse IgG2a or IgG2b) of immunoglobulin molecule.

As used herein, the term “Fc region” refers to the portion of an immunoglobulin formed by the Fc domains of its two heavy chains. The Fc region can be a wild-type Fc region (native Fc region) or a variant Fc region. A native Fc region is homodimeric. The Fc region can be derived from any native immunoglobulin. In some embodiments, the Fc region is formed from an IgA, IgD, IgE, or IgG heavy chain constant region. In some embodiments, the Fc region is formed from an IgG heavy chain constant region. In some embodiments, the IgG heavy chain is an IgG1, IgG2, IgG3, or IgG4 heavy chain constant region. In some embodiments, the Fc region is formed from an IgG1 heavy chain constant region. In some embodiments, the IgG1 heavy chain constant region comprises a G1m1(a), G1m2(x), G1m3(f), or G1m17(z) allotype. See, e.g., Jefferis and Lefranc, (2009) mAbs 1 (4): 332-338, and de Taeye et al., (2020) Front Immunol. 11:740, incorporated herein by reference in their entirety.

As used herein, the term “variant Fc region” refers to a variant of an Fc region with one or more alteration(s) relative to a native Fc region. Alterations can include amino acid substitutions, additions and/or deletions, linkage of additional moieties, and/or alteration of the native glycans. The term encompasses heterodimeric Fc regions where each of the constituent Fc domains is different. The term also encompasses single chain Fc regions where the constituent Fc domains are linked together by a linker moiety.

As used herein, the term “Fc domain” refers to the portion of a single immunoglobulin heavy chain comprising both the CH2 and CH3 domains of the antibody. In some embodiments, the Fc domain comprises at least a portion of a hinge (e.g., upper, middle, and/or lower hinge region) region, a CH2 domain, and a CH3 domain. In some embodiments, the Fc domain does not include the hinge region.

As used herein, the term “hinge region” refers to the portion of a heavy chain molecule that joins the CH1 domain to the CH2 domain. In some embodiments, the hinge region is at most, 70 amino acid residues in length. In some embodiments, this hinge region comprises approximately 11-17 amino acid residues and is flexible, thus allowing the two N-terminal antigen binding regions to move independently. In some embodiments, the hinge region is 12 amino acids in length. In some embodiments, the hinge region is 15 amino acids in length. In some embodiments, the hinge region is 62 amino acids in length. Hinge regions can be subdivided into three distinct domains: upper, middle, and lower hinge domains. The FcRn antagonist molecules of the instant disclosure can include all or any portion of a hinge region. In some embodiments, the hinge region is from an IgG1 antibody. In some embodiments, the hinge region comprises the amino acid sequence of EPKSCDKTHTCPPCP (SEQ ID NO: 23).

As used herein, the term “EU position” refers to the amino acid position in the EU numbering convention for the Fc region described in Edelman, G M et al., Proc. Natl. Acad. USA, 63, 78-85 (1969) and Kabat et al., in “Sequences of Proteins of Immunological Interest,” U.S. Dept. Health and Human Services, 5th edition, 1991.

As used herein, the term “antibody-mediated disorder” refers to any disorder wherein the symptoms of the disorder are caused by abnormal levels of one or more antibodies in a subject. As used herein, the term “autoantibody-mediated disorder” refers to any disease or disorder in which the underlying pathology is caused, at least in part, by pathogenic IgG autoantibodies.

As used herein, the term “treat,” “treating,” and “treatment” refer to therapeutic or preventative measures described herein. The methods of “treatment” employ administration of a polypeptide to a subject having a disease or disorder, or predisposed to having such a disease or disorder, in order to prevent, cure, delay, reduce the severity of, or ameliorate one or more symptoms of the disease or disorder or recurring disease or disorder, or in order to prolong the survival of a subject beyond that expected in the absence of such treatment.

As used herein, the term “effective amount” in the context of the administration of a therapy to a subject refers to the amount of a therapy that achieves a desired prophylactic or therapeutic effect.

As used herein, the term “dose” or “dosing” refers to an amount of an agent administered to a subject in a single administration.

As used herein, the term “subject” or “patient” or “participant” includes any human or non-human animal. In an embodiment, the subject or patient or participant is a human or non-human mammal. In an embodiment, the subject or patient or participant is a human.

As used herein, the term “about” or “approximately” when referring to a measurable value, such as a dosage, encompasses variations of ±5% of a given value or range.

As used herein, the term “molecular weight” can refer to a “predicted molecular weight” or an “observed molecular weight.” The “predicted molecular weight” of a protein is a sum of the molecular weights of all the amino acids in the protein. In certain circumstances the “predicted molecular weight” can differ from the “observed molecular weight” of a molecule. In some embodiments, these differences can occur in a protein because of changes in glycosylation, glycanation, ubiquitination, phosphorylation, or protein cleavage of the protein or complexes of additional proteins with a given protein.

### FcRn Antagonist Molecules

FcRn antagonist molecules disclosed herein comprise or consist of at least one Fc domain comprising or consisting of the amino acid sequence of SEQ ID NO:1, provided below.

TABLE 1

| | SEQ |
|---|---|
| Amino Acid Sequence | ID NO: |
| X₁X₂X₃X₄X₅X₆PPCPAPELLGGPSVFLFPPKPKDTLYITR | 1 |
| EPEVTCVVVDVSHEDPEVKFNWYVDGVEVHNAKTKPREEQY | |
| NSTYRVVSVLTVLHQDWLNGKEYKCKVSNKALPAPIEKTIS | |
| KAKGQPREPQVYTLPPSRDELTKNQVSLTCLVKGFYPSDIA | |
| VEWESNGQPENNYKTTPPVLDSDGSFFLYSKLTVDKSRWQQ | |
| GNVFSCSVMHEALKFHYTQKSLSLSPX₇X₈ | |

wherein:
X₁ is D or absent;
X₂ is K or absent;
X₃ is T or absent;
X₄ is H or absent;
X₅ is T or absent;
X₆ is C or absent;
X₇ is G or absent;
X₈ is K or absent.

In some embodiments, the FcRn antagonist molecules disclosed herein comprise or consist of a variant Fc region comprising or consisting of a dimer of a first Fc domain and a second Fc domain, wherein the amino acid sequence of the first and second Fc domain comprises or consists of the amino acid sequence of SEQ ID NO: 1.

The disclosure also provides a population of FcRn antagonist molecules, wherein FcRn antagonist molecules in the population comprise or consist of a variant Fc region comprising or consisting of a dimer of a first Fc domain and a second Fc domain, wherein the amino acid sequence of the first and second Fc domain comprises or consists of the amino acid sequence of SEQ ID NO: 1, provided that the population is not a homogeneous population of homodimeric FcRn antagonist molecules in which the amino acid sequence of both the first and the second Fc domain comprises or consists of the amino acid sequence of SEQ ID NO: 2, 3, 20, or 21, as set forth in Table 2.

TABLE 2

| Amino Acid Sequence | SEQ ID NO: |
|---|---|
| DKTHTCPPCPAPELLGGPSVFLFPPKPKDTLYITREPEVTCVVVDVSHEDP | 2 |
| EVKFNWYVDGVEVHNAKTKPREEQYNSTYRVVSVLTVLHQDWLNGKEYKCK | |
| VSNKALPAPIEKTISKAKGQPREPQVYTLPPSRDELTKNQVSLTCLVKGFY | |
| PSDIAVEWESNGQPENNYKTTPPVLDSDGSFFLYSKLTVDKSRWQQGNVFS | |
| CSVMHEALKFHYTQKSLSLSPGK | |
| DKTHTCPPCPAPELLGGPSVFLFPPKPKDTLYITREPEVTCVVVDVSHEDP | 3 |
| EVKFNWYVDGVEVHNAKTKPREEQYNSTYRVVSVLTVLHQDWLNGKEYKCK | |
| VSNKALPAPIEKTISKAKGQPREPQVYTLPPSRDELTKNQVSLTCLVKGFY | |
| PSDIAVEWESNGQPENNYKTTPPVLDSDGSFFLYSKLTVDKSRWQQGNVFS | |
| CSVMHEALKFHYTQKSLSLSPG | |
| CPPCPAPELLGGPSVFLFPPKPKDTLYITREPEVTCVVVDVSHEDPEVKFN | 20 |
| WYVDGVEVHNAKTKPREEQYNSTYRVVSVLTVLHQDWLNGKEYKCKVSNKA | |
| LPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVMH | |
| EALKFHYTQKSLSLSPG | |
| CPPCPAPELLGGPSVFLFPPKPKDTLYITREPEVTCVVVDVSHEDPEVKFN | 21 |
| WYVDGVEVHNAKTKPREEQYNSTYRVVSVLTVLHQDWLNGKEYKCKVSNKA | |
| LPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVMH | |
| EALKFHYTQKSLSLSPGK | |

In some embodiments, each FcRn antagonist molecule in the population comprises or consists of an amino acid sequence at least 70%, 75%, 80%, 85%, 90%, 95%, 96%, 97%, 98%, or 99% identical to the amino acid sequence of a variant Fc region comprising or consisting of a dimer of a first Fc domain and a second Fc domain, wherein the amino acid sequence of the first and second Fc domain comprises or consists of the amino acid sequence of SEQ ID NOs: 2-22, provided that the population is not a homogeneous population of homodimeric FcRn antagonist molecules in which the amino acid sequence of both the first and the second Fc domain comprises or consists of the amino acid sequence of SEQ ID NO: 2, 3, 20, or 21. In some embodiments, each FcRn antagonist molecule in the population comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 2-22, provided that the population is not a homogeneous population of homodimeric FcRn antagonist molecules in which the amino acid sequence of both the first and the second Fc domain comprises or consists of the amino acid sequence of SEQ ID NO: 2, 3, 20, or 21. The amino acid sequences of SEQ ID NOs: 4-19 and 22 as set forth in Table 3.

TABLE 3

| | SEQ |
|---|---|
| Amino Acid Sequence | ID NO: |
| DKTHTCPPCPAPELLGGPSVFLFPPKPKDTLYITREPEVTCVVVDVSHEDP | 4 |
| EVKFNWYVDGVEVHNAKTKPREEQYNSTYRVVSVLTVLHQDWLNGKEYKCK | |
| VSNKALPAPIEKTISKAKGQPREPQVYTLPPSRDELTKNQVSLTCLVKGFY | |
| PSDIAVEWESNGQPENNYKTTPPVLDSDGSFFLYSKLTVDKSRWQQGNVFS | |
| CSVMHEALKFHYTQKSLSLSP | |
| KTHTCPPCPAPELLGGPSVFLFPPKPKDTLYITREPEVTCVVVDVSHEDPE | 5 |
| VKFNWYVDGVEVHNAKTKPREEQYNSTYRVVSVLTVLHQDWLNGKEYKCKV | |
| SNKALPAPIEKTISKAKGQPREPQVYTLPPSRDELTKNQVSLTCLVKGFYP | |
| SDIAVEWESNGQPENNYKTTPPVLDSDGSFFLYSKLTVDKSRWQQGNVFSC | |
| SVMHEALKFHYTQKSLSLSPGK | |
| KTHTCPPCPAPELLGGPSVFLFPPKPKDTLYITREPEVTCVVVDVSHEDPE | 6 |
| VKFNWYVDGVEVHNAKTKPREEQYNSTYRVVSVLTVLHQDWLNGKEYKCKV | |
| SNKALPAPIEKTISKAKGQPREPQVYTLPPSRDELTKNQVSLTCLVKGFYP | |
| SDIAVEWESNGQPENNYKTTPPVLDSDGSFFLYSKLTVDKSRWQQGNVFSC | |
| SVMHEALKFHYTQKSLSLSPG | |
| KTHTCPPCPAPELLGGPSVFLFPPKPKDTLYITREPEVTCVVVDVSHEDPE | 7 |
| VKFNWYVDGVEVHNAKTKPREEQYNSTYRVVSVLTVLHQDWLNGKEYKCKV | |
| SNKALPAPIEKTISKAKGQPREPQVYTLPPSRDELTKNQVSLTCLVKGFYP | |
| SDIAVEWESNGQPENNYKTTPPVLDSDGSFFLYSKLTVDKSRWQQGNVFSC | |
| SVMHEALKFHYTQKSLSLSP | |
| THTCPPCPAPELLGGPSVFLFPPKPKDTLYITREPEVTCVVVDVSHEDPEV | 8 |
| KFNWYVDGVEVHNAKTKPREEQYNSTYRVVSVLTVLHQDWLNGKEYKCKVS | |
| NKALPAPIEKTISKAKGQPREPQVYTLPPSRDELTKNQVSLTCLVKGFYPS | |
| DIAVEWESNGQPENNYKTTPPVLDSDGSFFLYSKLTVDKSRWQQGNVFSCS | |
| VMHEALKFHYTQKSLSLSPGK | |
| THTCPPCPAPELLGGPSVFLFPPKPKDTLYITREPEVTCVVVDVSHEDPEV | 9 |
| KFNWYVDGVEVHNAKTKPREEQYNSTYRVVSVLTVLHQDWLNGKEYKCKVS | |
| NKALPAPIEKTISKAKGQPREPQVYTLPPSRDELTKNQVSLTCLVKGFYPS | |
| DIAVEWESNGQPENNYKTTPPVLDSDGSFFLYSKLTVDKSRWQQGNVFSCS | |
| VMHEALKFHYTQKSLSLSPG | |
| THTCPPCPAPELLGGPSVFLFPPKPKDTLYITREPEVTCVVVDVSHEDPEV | 10 |
| KFNWYVDGVEVHNAKTKPREEQYNSTYRVVSVLTVLHQDWLNGKEYKCKVS | |
| NKALPAPIEKTISKAKGQPREPQVYTLPPSRDELTKNQVSLTCLVKGFYPS | |
| DIAVEWESNGQPENNYKTTPPVLDSDGSFFLYSKLTVDKSRWQQGNVFSCS | |
| VMHEALKFHYTQKSLSLSP | |
| TCPPCPAPELLGGPSVFLFPPKPKDTLYITREPEVTCVVVDVSHEDPEVKF | 11 |
| NWYVDGVEVHNAKTKPREEQYNSTYRVVSVLTVLHQDWLNGKEYKCKVSNK | |
| ALPAPIEKTISKAKGQPREPQVYTLPPSRDELTKNQVSLTCLVKGFYPSDI | |
| AVEWESNGQPENNYKTTPPVLDSDGSFFLYSKLTVDKSRWQQGNVFSCSVM | |
| HEALKFHYTQKSLSLSPGK | |
| TCPPCPAPELLGGPSVFLFPPKPKDTLYITREPEVTCVVVDVSHEDPEVKF | 12 |
| NWYVDGVEVHNAKTKPREEQYNSTYRVVSVLTVLHQDWLNGKEYKCKVSNK | |
| ALPAPIEKTISKAKGQPREPQVYTLPPSRDELTKNQVSLTCLVKGFYPSDI | |
| AVEWESNGQPENNYKTTPPVLDSDGSFFLYSKLTVDKSRWQQGNVFSCSVM | |
| HEALKFHYTQKSLSLSPG | |
| TCPPCPAPELLGGPSVFLFPPKPKDTLYITREPEVTCVVVDVSHEDPEVKF | 13 |
| NWYVDGVEVHNAKTKPREEQYNSTYRVVSVLTVLHQDWLNGKEYKCKVSNK | |
| ALPAPIEKTISKAKGQPREPQVYTLPPSRDELTKNQVSLTCLVKGFYPSDI | |
| AVEWESNGQPENNYKTTPPVLDSDGSFFLYSKLTVDKSRWQQGNVFSCSVM | |
| HEALKFHYTQKSLSLSP | |
| PPCPAPELLGGPSVFLFPPKPKDTLYITREPEVTCVVVDVSHEDPEVKFNW | 14 |
| YVDGVEVHNAKTKPREEQYNSTYRVVSVLTVLHQDWLNGKEYKCKVSNKAL | |
| PAPIEKTISKAKGQPREPQVYTLPPSRDELTKNQVSLTCLVKGFYPSDIAV | |
| EWESNGQPENNYKTTPPVLDSDGSFFLYSKLTVDKSRWQQGNVFSCSVMHE | |
| ALKFHYTQKSLSLSPGK | |
| PPCPAPELLGGPSVFLFPPKPKDTLYITREPEVTCVVVDVSHEDPEVKFNW | 15 |
| YVDGVEVHNAKTKPREEQYNSTYRVVSVLTVLHQDWLNGKEYKCKVSNKAL | |
| PAPIEKTISKAKGQPREPQVYTLPPSRDELTKNQVSLTCLVKGFYPSDIAV | |
| EWESNGQPENNYKTTPPVLDSDGSFFLYSKLTVDKSRWQQGNVFSCSVMHE | |
| ALKFHYTQKSLSLSPG | |
| PPCPAPELLGGPSVFLFPPKPKDTLYITREPEVTCVVVDVSHEDPEVKFNW | 16 |
| YVDGVEVHNAKTKPREEQYNSTYRVVSVLTVLHQDWLNGKEYKCKVSNKAL | |
| PAPIEKTISKAKGQPREPQVYTLPPSRDELTKNQVSLTCLVKGFYPSDIAV | |
| EWESNGQPENNYKTTPPVLDSDGSFFLYSKLTVDKSRWQQGNVFSCSVMHE | |
| ALKFHYTQKSLSLSP | |
| HTCPPCPAPELLGGPSVFLFPPKPKDTLYITREPEVTCVVVDVSHEDPEVK | 17 |
| FNWYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSD | |
| IAVEWESNGQPENNYKTTPPVLDSDGSFFLYSKLTVDKSRWQQGNVFSCSV | |
| MHEALKFHYTQKSLSLSPGK | |
| HTCPPCPAPELLGGPSVFLFPPKPKDTLYITREPEVTCVVVDVSHEDPEVK | 18 |
| FNWYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSD | |
| IAVEWESNGQPENNYKTTPPVLDSDGSFFLYSKLTVDKSRWQQGNVFSCSV | |
| MHEALKFHYTQKSLSLSPG | |
| HTCPPCPAPELLGGPSVFLFPPKPKDTLYITREPEVTCVVVDVSHEDPEVK | 19 |
| FNWYVDGVEVHNAKTKPREEQYNSTYRVVSVLTVLHQDWLNGKEYKCKVSN | |
| KALPAPIEKTISKAKGQPREPQVYTLPPSRDELTKNQVSLTCLVKGFYPSD | |
| IAVEWESNGQPENNYKTTPPVLDSDGSFFLYSKLTVDKSRWQQGNVFSCSV | |
| MHEALKFHYTQKSLSLSP | |
| CPPCPAPELLGGPSVFLFPPKPKDTLYITREPEVTCVVVDVSHEDPEVKFN | 22 |
| WYVDGVEVHNAKTKPREEQYNSTYRVVSVLTVLHQDWLNGKEYKCKVSNKA | |
| LPAPIEKTISKAKGQPREPQVYTLPPSRDELTKNQVSLTCLVKGFYPSDIA | |
| VEWESNGQPENNYKTTPPVLDSDGSFFLYSKLTVDKSRWQQGNVFSCSVMH | |
| EALKFHYTQKSLSLSP | |

Embodiments for a variety of FcRn antagonist molecules are shown below in Table 4. The amino acid sequence of each member of the dimer of a first Fc domain (I) and a second Fc domain (II) are shown by SEQ ID NO. So, for example, I=2, and II=3 shown in the second cell of Table 4 represents an FcRn antagonist molecule in the population that comprises or consists of a variant Fc region comprising or consisting of a dimer of a first Fc domain (I) and a second Fc domain (II), wherein the amino acid sequence of the first and second Fc domain comprises or consists of the amino acid sequence of SEQ ID NOs: 2 and 3, respectively.

TABLE 4
SEQ ID NOs of first (I) and second (II) Fc domains in variant Fc region dimers

| I | II | I | II | I | II | I | II | I | II | I | II | I | II | I | II |
|---|---|---|---|---|---|---|---|---|---|---|---|---|---|---|---|
| 2 | 2 | 2 | 3 | 2 | 4 | 2 | 5 | 2 | 6 | 2 | 7 | 2 | 8 | 2 | 9 |
| 2 | 10 | 2 | 11 | 2 | 12 | 2 | 13 | 2 | 14 | 2 | 15 | 2 | 16 | 2 | 17 |
| 2 | 18 | 2 | 19 | 2 | 20 | 2 | 21 | 2 | 22 | 3 | 3 | 3 | 4 | 3 | 5 |
| 3 | 6 | 3 | 7 | 3 | 8 | 3 | 9 | 3 | 10 | 3 | 11 | 3 | 12 | 3 | 13 |
| 3 | 14 | 3 | 15 | 3 | 16 | 3 | 17 | 3 | 18 | 3 | 19 | 3 | 20 | 3 | 21 |
| 3 | 22 | 4 | 4 | 4 | 5 | 4 | 6 | 4 | 7 | 4 | 8 | 4 | 9 | 4 | 10 |
| 4 | 11 | 4 | 12 | 4 | 13 | 4 | 14 | 4 | 15 | 4 | 16 | 4 | 17 | 4 | 18 |
| 4 | 19 | 4 | 20 | 4 | 21 | 4 | 22 | 5 | 5 | 5 | 6 | 5 | 7 | 5 | 8 |
| 5 | 9 | 5 | 10 | 5 | 11 | 5 | 12 | 5 | 13 | 5 | 14 | 5 | 15 | 5 | 16 |
| 5 | 17 | 5 | 18 | 5 | 19 | 5 | 20 | 5 | 21 | 5 | 22 | 6 | 6 | 6 | 7 |
| 6 | 8 | 6 | 9 | 6 | 10 | 6 | 11 | 6 | 12 | 6 | 13 | 6 | 14 | 6 | 15 |
| 6 | 16 | 6 | 17 | 6 | 18 | 6 | 19 | 6 | 20 | 6 | 21 | 6 | 22 | 7 | 7 |
| 7 | 8 | 7 | 9 | 7 | 10 | 7 | 11 | 7 | 12 | 7 | 13 | 7 | 14 | 7 | 15 |
| 7 | 16 | 7 | 17 | 7 | 18 | 7 | 19 | 7 | 20 | 7 | 21 | 7 | 22 | 8 | 8 |
| 8 | 9 | 8 | 10 | 8 | 11 | 8 | 12 | 8 | 13 | 8 | 14 | 8 | 15 | 8 | 16 |
| 8 | 17 | 8 | 18 | 8 | 19 | 8 | 20 | 8 | 21 | 8 | 22 | 9 | 9 | 9 | 10 |
| 9 | 11 | 9 | 12 | 9 | 13 | 9 | 14 | 9 | 15 | 9 | 16 | 9 | 17 | 9 | 18 |
| 9 | 19 | 9 | 20 | 9 | 21 | 9 | 22 | 10 | 10 | 10 | 11 | 10 | 12 | 10 | 13 |
| 10 | 14 | 10 | 15 | 10 | 16 | 10 | 17 | 10 | 18 | 10 | 19 | 10 | 20 | 10 | 21 |
| 10 | 22 | 11 | 11 | 11 | 12 | 11 | 13 | 11 | 14 | 11 | 15 | 11 | 16 | 11 | 17 |
| 11 | 18 | 11 | 19 | 11 | 20 | 11 | 21 | 11 | 22 | 12 | 12 | 12 | 13 | 12 | 14 |
| 12 | 15 | 12 | 16 | 12 | 17 | 12 | 18 | 12 | 19 | 12 | 20 | 12 | 21 | 12 | 22 |
| 13 | 13 | 13 | 14 | 13 | 15 | 13 | 16 | 13 | 17 | 13 | 18 | 13 | 19 | 13 | 20 |
| 13 | 21 | 13 | 22 | 14 | 14 | 14 | 15 | 14 | 16 | 14 | 17 | 14 | 18 | 14 | 19 |
| 14 | 20 | 14 | 21 | 14 | 22 | 15 | 15 | 15 | 16 | 15 | 17 | 15 | 18 | 15 | 19 |
| 15 | 20 | 15 | 21 | 15 | 22 | 16 | 16 | 16 | 17 | 16 | 18 | 16 | 19 | 16 | 20 |
| 16 | 21 | 16 | 22 | 17 | 17 | 17 | 18 | 17 | 19 | 17 | 20 | 17 | 21 | 17 | 22 |
| 18 | 18 | 18 | 19 | 18 | 20 | 18 | 21 | 18 | 22 | 19 | 19 | 19 | 20 | 19 | 21 |
| 19 | 22 | 20 | 20 | 20 | 21 | 20 | 22 | 21 | 21 | 21 | 22 | 22 | 22 | | |

In some embodiments, a population of FcRn antagonist molecules described herein is not a homogeneous population of homodimeric FcRn antagonist molecules in which the amino acid sequence of both the first and the second Fc domain consists of the amino acid sequence of SEQ ID NO: 2, 3, 20, or 21. In some embodiments, populations of FcRn antagonist molecules described herein include homodimeric FcRn antagonist molecules in which the amino acid sequence of both the first and the second Fc domain consists of the amino acid sequence of SEQ ID NO: 2, 3, 20, or 21, however, these populations further comprise other FcRn antagonist molecules. In some embodiments, these other FcRn antagonist molecules are presented in Table 4 above, i.e., excluding when: I=2 and II=2; I=3 and II=3, I=20 and II=20, and I=21 and II=21.

In some embodiments, each FcRn antagonist molecule in the population comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, or 99% identical to the amino acid sequence of the amino acid sequence of SEQ ID NOs: 3 and 12, respectively. In some embodiments, each FcRn antagonist molecule in the population comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 3 and 12, respectively. In some embodiments, each FcRn antagonist molecule in the population comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, or 99% identical to the amino acid sequence of SEQ ID NOs: 3 and 9, respectively. In some embodiments, each FcRn antagonist molecule in the population comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 3 and 9, respectively. In some embodiments, each FcRn antagonist molecule in the population comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, or 99% identical to the amino acid sequence of SEQ ID NOs: 2 and 3, respectively. In some embodiments, each FcRn antagonist molecule in the population comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 2 and 3, respectively. In some embodiments, each FcRn antagonist molecule in the population comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, or 99% identical to the amino acid sequence of SEQ ID NOs: 3 and 6, respectively. In some embodiments, each FcRn antagonist molecule in the population comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 3 and 6, respectively.

In some embodiments, each FcRn antagonist molecule in the population comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, or 99% identical to the amino acid sequence of SEQ ID NO: 4. In some embodiments, each FcRn antagonist molecule in the population comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, or 99% identical to the amino acid sequence of the amino acid sequence of SEQ ID NO: 5. In some embodiments, each FcRn antagonist molecule in the population comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, or 99% identical to the amino acid sequence of SEQ ID NO: 6. In some embodiments, each FcRn antagonist molecule in the population comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, or 99% identical to the amino acid sequence of SEQ ID NO: 7. In some embodiments, each FcRn antagonist molecule in the population comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, or 99% identical to the amino acid sequence of SEQ ID NO: 8. In some embodiments, each FcRn antagonist molecule in the population comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, or 99% identical to the amino acid sequence of SEQ ID NO: 9. In some embodiments, each FcRn antagonist molecule in the population comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, or 99% identical to the amino acid sequence of SEQ ID NO: 10. In some embodiments, each FcRn antagonist molecule in the population comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, or 99% identical to the amino acid sequence of SEQ ID NO: 11. In some embodiments, each FcRn antagonist molecule in the population comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, or 99% identical to the amino acid sequence of SEQ ID NO: 12. In some embodiments, each FcRn antagonist molecule in the population comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, or 99% identical to the amino acid sequence of SEQ ID NO: 13. In some embodiments, each FcRn antagonist molecule in the population comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, or 99% identical to the amino acid sequence of SEQ ID NO: 14. In some embodiments, each FcRn antagonist molecule in the population comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, or 99% identical to the amino acid sequence of SEQ ID NO: 15. In some embodiments, each FcRn antagonist molecule in the population comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, or 99% identical to the amino acid sequence of SEQ ID NO: 16. In some embodiments, each FcRn antagonist molecule in the population comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, or 99% identical to the amino acid sequence of SEQ ID NO: 17. In some embodiments, each FcRn antagonist molecule in the population comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, or 99% identical to the amino acid sequence of SEQ ID NO: 18. In some embodiments, each FcRn antagonist molecule in the population comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, or 99% identical to the amino acid sequence of SEQ ID NO: 19. In some embodiments, each FcRn antagonist molecule in the population comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, or 99% identical to the amino acid sequence of SEQ ID NO: 22.

In some embodiments, each FcRn antagonist molecule in the population comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 4. In some embodiments, each FcRn antagonist molecule in the population comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 5. In some embodiments, each FcRn antagonist molecule in the population comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 6. In some embodiments, each FcRn antagonist molecule in the population comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 7. In some embodiments, each FcRn antagonist molecule in the population comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 8. In some embodiments, each FcRn antagonist molecule in the population comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 9. In some embodiments, each FcRn antagonist molecule in the population comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 10. In some embodiments, each FcRn antagonist molecule in the population comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 11. In some embodiments, each FcRn antagonist molecule in the population comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 12. In some embodiments, each FcRn antagonist molecule in the population comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 13. In some embodiments, each FcRn antagonist molecule in the population comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 14. In some embodiments, each FcRn antagonist molecule in the population comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 15. In some embodiments, each FcRn antagonist molecule in the population comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 16. In some embodiments, each FcRn antagonist molecule in the population comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 17. In some embodiments, each FcRn antagonist molecule in the population comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 18. In some embodiments, each FcRn antagonist molecule in the population comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 19. In some embodiments, each FcRn antagonist molecule in the population comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 22.

In some embodiments, the FcRn antagonist molecules in the population comprise glycanation on one or both of their Fc domains. In some embodiments, the FcRn antagonist molecules in the population comprise glycanation at EU position 297 on one or both of their Fc domains. In some embodiments, the glycanation comprises an N-glycan. In some embodiments, the N-glycan comprises, a G0F N-glycan, G1F N-glycan, G2F N-glycan, or G0 N-glycan.

In some embodiments, the FcRn antagonist molecules comprise or consist of a variant Fc region comprising or consisting of a dimer of a first Fc domain and a second Fc domain, wherein one of the Fc domains is glycanated. In some embodiments, the glycanation comprises an N-glycan. In some embodiments, the N-glycan comprises, a G0F N-glycan, G1F N-glycan, G2F N-glycan, or G0 N-glycan.

In some embodiments, the FcRn antagonist molecules comprise or consist of a variant Fc region comprising or consisting of a dimer of a first Fc domain and a second Fc domain, wherein both of the Fc domains are glycanated. In some embodiments, the glycanation comprises an N-glycan. In some embodiments, the N-glycan comprises, a G0F N-glycan, G1F N-glycan, G2F N-glycan, or G0 N-glycan. In some embodiments, the Fc domains comprise a G0F N-glycan. In some embodiments, the Fc domains comprise a G1F N-glycan. In some embodiments, the Fc domains comprise a G2F N-glycan. In some embodiments, the Fc domains comprise a G0 N-glycan.

In some embodiments, the first Fc domain and the second Fc domain have different N-glycanations at EU position 297. In some embodiments, the first Fc domain comprises a GOF N-glycan, and the second Fc domain comprises a G1F N-glycan. In some embodiments, the first Fc domain comprises a G0F N-glycan, and the second Fc domain comprises a G2F N-glycan. In some embodiments, the first Fc domain comprises a G0F N-glycan, and the second Fc domain comprises a G0 N-glycan. In some embodiments, the first Fc domain comprises a G1F N-glycan, and the second Fc domain comprises a G2F N-glycan. In some embodiments, the first Fc domain comprises a G1F N-glycan, and the second Fc domain comprises a G2F+NANA N-glycan. In some embodiments, the first Fc domain comprises a G2F N-glycan, and the second Fc domain comprises a G2F+2×NANA N-glycan. In some embodiments, the first Fc domain comprises a G1F N-glycan, and the second Fc domain comprises a G0 N-glycan. In some embodiments, the first Fc domain comprises a G2F N-glycan, and the second Fc domain comprises a G0 N-glycan. In some embodiments, one or more of the amino acids of the FcRn antagonist molecules is modified. In some embodiments, an asparagine residue is deaminated. In some embodiments, a methionine residue is oxidized. In some embodiments, a tryptophan residue is oxidized. In some embodiments, both a methionine and a tryptophan residue are oxidized.

In some embodiments, the population of FcRn antagonist molecules comprises or consists of multiple subpopulations of FcRn antagonist molecules. In some embodiments, the population of FcRn antagonist molecules comprises or consists of 2, 3, 4, 5, 6, 7, 8, 9, 10, 11, 12, 13, 14, 15, 16, 17, 18, 19, or 20 subpopulations. In some embodiments, the population of FcRn antagonist molecules comprises or consists of 2, 3, 4, 5, 6, 7, 8, 9, 10, or 11 subpopulations.

In some embodiments, a first subpopulation of FcRn antagonist molecules comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, 99%, or 100% identical to the amino acid sequence of SEQ ID NO: 3.

In some embodiments, a second subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 3 and 12, respectively.

In some embodiments, a third subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 3 and 9, respectively.

In some embodiments, a fourth subpopulation of FcRn antagonist molecules comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, 99%, or 100% identical to the amino acid sequence of SEQ ID NO: 3, and wherein two asparagine residues in each FcRn antagonist molecule in the fourth subpopulation are deaminated.

In some embodiments, a fifth subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 3 and 9, respectively, and wherein one asparagine residue in each FcRn antagonist molecule in the fifth subpopulation are deaminated.

In some embodiments, a sixth subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 2 and 3, respectively.

In some embodiments, a seventh subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 2 and 3, respectively, and wherein one methionine residue or one tryptophan residue in each FcRn antagonist molecule in the seventh subpopulation is oxidized.

In some embodiments, an eighth subpopulation of FcRn antagonist molecules comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, 99%, or 100% identical to the amino acid sequence of SEQ ID NO: 2.

In some embodiments, a ninth subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 3 and 6, respectively.

In some embodiments, a tenth subpopulation of FcRn antagonist molecules comprises or consists of a variant Fc region comprising or consisting of a dimer of a first Fc domain and a second Fc domain, wherein the amino acid sequence of the first and second Fc domain comprises or consists of an amino acid sequence at least 70%, 75%, 80%, 85%, 90%, 95%, 96%, 97%, 98%, 99%, or 100% identical to the amino acid sequence of SEQ ID NOs: 2 and 3, respectively, and wherein one methionine residue or one tryptophan residue in each FcRn antagonist molecule in the tenth subpopulation is oxidized.

In some embodiments, an eleventh subpopulation of FcRn antagonist molecules comprises or consists of a variant Fc region comprising or consisting of a dimer of a first Fc domain and a second Fc domain, wherein the amino acid sequence of both the first and second Fc domain comprises or consists of an amino acid sequence at least 70%, 75%, 80%, 85%, 90%, 95%, 96%, 97%, 98%, 99%, or 100% identical to the amino acid sequence of SEQ ID NO: 3, and wherein one methionine residue or one tryptophan residue in each FcRn antagonist molecule in the eleventh subpopulation is oxidized.

In some embodiments, a first subpopulation of FcRn antagonist molecules comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 3.

In some embodiments, a second subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 3 and 12, respectively.

In some embodiments, a third subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 3 and 9, respectively.

In some embodiments, a fourth subpopulation of FcRn antagonist molecules comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 3, and wherein two asparagine residues in each FcRn antagonist molecule in the fourth subpopulation are deaminated.

In some embodiments, a fifth subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 3 and 9, respectively, and wherein one asparagine residue in each FcRn antagonist molecule in the fifth subpopulation is deaminated.

In some embodiments, a sixth subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 2 and 3, respectively.

In some embodiments, a seventh subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 2 and 3, respectively, and wherein one methionine residue or one tryptophan residue in each FcRn antagonist molecule in the seventh subpopulation is oxidized.

In some embodiments, an eighth subpopulation of FcRn antagonist molecules comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 2.

In some embodiments, a ninth subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 3 and 6, respectively.

In some embodiments, a tenth subpopulation of FcRn antagonist molecules comprises or consists of a variant Fc region comprising or consisting of a dimer of a first Fc domain and a second Fc domain, wherein the amino acid sequence of the first and second Fc domain comprises or consists of the amino acid sequence of SEQ ID NOs: 2 and 3, respectively, and wherein one methionine residue or one tryptophan residue in each FcRn antagonist molecule in the tenth subpopulation is oxidized.

In some embodiments, an eleventh subpopulation of FcRn antagonist molecules comprises or consists of a variant Fc region comprising or consisting of a dimer of a first Fc domain and a second Fc domain, wherein the amino acid sequence of both the first and second Fc domain comprises or consists of the amino acid sequence of SEQ ID NO: 3, and wherein one methionine residue or one tryptophan residue in each FcRn antagonist molecule in the eleventh subpopulation is oxidized.

In some embodiments, the population of FcRn antagonist molecules comprises or consists of the first subpopulation combined with one of the second, third, fourth, fifth, sixth, seventh, eighth, ninth, tenth, or eleventh subpopulations. In some embodiments, the population of FcRn antagonist molecules comprises or consists of the first subpopulation combined with two of the second, third, fourth, fifth, sixth, seventh, eighth, ninth, tenth, or eleventh subpopulations. In some embodiments, the population of FcRn antagonist molecules comprises or consists of the first subpopulation combined with three of the second, third, fourth, fifth, sixth, seventh, eighth, ninth, tenth, or eleventh subpopulations. In some embodiments, the population of FcRn antagonist molecules comprises or consists of the first subpopulation combined with four of the second, third, fourth, fifth, sixth, seventh, eighth, ninth, tenth, or eleventh subpopulations. In some embodiments, the population of FcRn antagonist molecules comprises or consists of the first subpopulation combined with five of the second, third, fourth, fifth, sixth, seventh, eighth, ninth, tenth, or eleventh subpopulations. In some embodiments, the population of FcRn antagonist molecules comprises or consists of the first subpopulation combined with six of the second, third, fourth, fifth, sixth, seventh, eighth, ninth, tenth, or eleventh subpopulations. In some embodiments, the population of FcRn antagonist molecules comprises or consists of the first subpopulation combined with seven of the second, third, fourth, fifth, sixth, seventh, eighth, ninth, tenth, or eleventh subpopulations. In some embodiments, the population of FcRn antagonist molecules comprises or consists of the first subpopulation combined with eight of the second, third, fourth, fifth, sixth, seventh, eighth, ninth, tenth, or eleventh subpopulations. In some embodiments, the population of FcRn antagonist molecules comprises or consists of the first subpopulation combined with nine of the second, third, fourth, fifth, sixth, seventh, eighth, ninth, tenth, or eleventh subpopulations. In some embodiments, the population of FcRn antagonist molecules comprises or consists of the first subpopulation combined with all of the second, third, fourth, fifth, sixth, seventh, eighth, ninth, tenth, or eleventh subpopulations.

In some embodiments, the population comprises or consists of the first and second subpopulations. In some embodiments, the population comprises or consists of the first and third subpopulations. In some embodiments, the population comprises or consists of the first and fourth subpopulations. In some embodiments, the population comprises or consists of the first and fifth subpopulations. In some embodiments, the population comprises or consists of the first and sixth subpopulations. In some embodiments, the population comprises or consists of the first and seventh subpopulations. In some embodiments, the population comprises or consists of the first and eighth subpopulations. In some embodiments, the population comprises or consists of the first and ninth subpopulations. In some embodiments, the population comprises or consists of the first and tenth subpopulations. In some embodiments, the population comprises or consists of the first and eleventh subpopulations. In some embodiments, the populations listed above further comprise or consist of 1, 2, 3, 4, 5, 6, 7, 8, or 9 additional subpopulations. In some embodiments, these additional subpopulations are one or more of those described above.

In some embodiments, the population comprises or consists of the first and seventh, ninth or eleventh subpopulations. In some embodiments, the population comprises or consists of the first, seventh, ninth and eleventh subpopulations.

In some embodiments, the first subpopulation makes up at least 40%, at least 45%, at least 50%, at least 55%, at least 60%, at least 65%, at least 70%, at least 75%, at least 80%, at least 85%, or at least 90% of the population of FcRn antagonist molecules. In some embodiments, the first subpopulation makes up about 40%, about 45%, about 50%, about 55%, about 60%, about 65%, about 70%, about 75%, about 80%, about 85%, or about 90% of the population of FcRn antagonist molecules. In some embodiments, the first subpopulation makes up 40%, 45%, 50%, 55%, 60%, 65%, 70%, 75%, 80%, 85%, or 90% of the population of FcRn antagonist molecules. In some embodiments, the first subpopulation makes up 40%-90%, 50%-80%, or 55%-70% of the population of FcRn antagonist molecules. In some embodiments, the first subpopulation makes up 56.9%-68.3% or 59.5%-67.9% of the population of FcRn antagonist molecules.

In some embodiments, the second subpopulation makes up less than 3.0%, less than 2.5%, less than 2.0%, less than 1.5%, less than 1%, or less than 0.5% of the population of FcRn antagonist molecules. In some embodiments, the second subpopulation makes up about 3.0%, about 2.5%, about 2.0%, about 1.5%, about 1%, or about 0.5% of the population of FcRn antagonist molecules. In some embodiments, the second subpopulation makes up 3.0%, 2.5%, 2.0%, 1.5%, 1%, or 0.5% of the population of FcRn antagonist molecules. In some embodiments, the second subpopulation makes up 0.5%-3.0%, 1.0%-2.5%, or 1.0%-2.0% of the population of FcRn antagonist molecules. In some embodiments, the second subpopulation makes up 0.8%-2.0% or 0.8%-2.1% of the population of FcRn antagonist molecules.

In some embodiments, the third subpopulation makes up less than 3.0%, less than 2.5%, less than 2.0%, less than 1.5%, less than 1%, or less than 0.5% of the population of FcRn antagonist molecules. In some embodiments, the third subpopulation makes up about 3.0%, about 2.5%, about 2.0%, about 1.5%, about 1%, or about 0.5% of the population of FcRn antagonist molecules. In some embodiments, the third subpopulation makes up 3.0%, 2.5%, 2.0%, 1.5%, 1%, or 0.5% of the population of FcRn antagonist molecules. In some embodiments, the third subpopulation makes up 0.5%-3.0%, 1.0%-2.5%, or 1.0%-2.0% of the population of FcRn antagonist molecules. In some embodiments, the third subpopulation makes up 1.1%-2.1% or 1.0%-1.9% of the population of FcRn antagonist molecules.

In some embodiments, the fourth subpopulation makes up less than 5%, less than 4%, less than 3%, less than 2%, or less than 1% of the population of FcRn antagonist molecules. In some embodiments, the fourth subpopulation makes up about 5%, about 4%, about 3%, about 2%, or about 1% of the population of FcRn antagonist molecules. In some embodiments, the fourth subpopulation makes up 5%, 4%, 3%, 2%, or 1% of the population of FcRn antagonist molecules. In some embodiments, the fourth subpopulation makes up 1%-5%, 2%-4%, or 2%-3% of the population of FcRn antagonist molecules. In some embodiments, the fourth subpopulation makes up 2.1%-3.2% or 2.0%-3.1% of the population of FcRn antagonist molecules.

In some embodiments, the fifth subpopulation makes up less than 12%, less than 11%, less than 10%, less than 9%, less than 8%, less than 7%, less than 6%, or less than 5% of the population of FcRn antagonist molecules. In some embodiments, the fifth subpopulation makes up about 12%, about 11%, about 10%, about 9%, about 8%, about 7%, about 6%, or about 5% of the population of FcRn antagonist molecules. In some embodiments, the fifth subpopulation makes up 12%, 11%, 10%, 9%, 8%, 7%, 6%, or 5% of the population of FcRn antagonist molecules. In some embodiments, the fifth subpopulation makes up 5%-12%, 6%-10%, or 7%-8% of the population of FcRn antagonist molecules. In some embodiments, the fifth subpopulation makes up 6.8%-9.4% or 6.9%-8.7% of the population of FcRn antagonist molecules.

In some embodiments, the sixth subpopulation makes up less than 17%, less than 16%, less than 15%, less than 14%, less than 13%, less than 12%, less than 11%, less than 10%, less than 9%, less than 8%, less than 7%, or less than 6% of the population of FcRn antagonist molecules. In some embodiments, the sixth subpopulation makes up about 17%, about 16%, about 15%, about 14%, about 13%, about 12%, about 11%, about 10%, about 9%, about 8%, about 7%, or about 6% of the population of FcRn antagonist molecules. In some embodiments, the sixth subpopulation makes up 17%, 16%, 15%, 14%, 13%, 12%, 11%, 10%, 9%, 8%, 7%, or 6% of the population of FcRn antagonist molecules. In some embodiments, the sixth subpopulation makes up 7%-17%, 10%-15%, or 11%-12% of the population of FcRn antagonist molecules. In some embodiments, the sixth subpopulation makes up 7.0%-14.0% or 10.0%-14.4% of the population of FcRn antagonist molecules.

In some embodiments, the seventh subpopulation makes up less than 6.0%, less than 5.5%, less than 5.0%, less than 4.5%, less than 4.0%, less than 3.5%, less than 3.0%, less than 2.5%, less than 2.0%, less than 1.5%, less than 1%, or less than 0.5% of the population of FcRn antagonist molecules. In some embodiments, the seventh subpopulation makes up about 6.0%, about 5.5%, about 5.0%, about 4.5%, about 4.0%, about 3.5%, about 3.0%, about 2.5%, about 2.0%, about 1.5%, about 1%, or about 0.5% of the population of FcRn antagonist molecules. In some embodiments, the seventh subpopulation makes up 6.0%, 5.5%, 5.0%, 4.5%, 4.0%, 3.5%, 3.0%, 2.5%, 2.0%, 1.5%, 1%, or 0.5% of the population of FcRn antagonist molecules. In some embodiments, the seventh subpopulation makes up 0.5%-5.5%, 1.0%-3.0%, or 1.5%-2.5% of the population of FcRn antagonist molecules. In some embodiments, the seventh subpopulation makes up 1.5%-5.5% or 1.4%-4.9% of the population of FcRn antagonist molecules.

In some embodiments, the eighth subpopulation makes up less than 7.5%, less than 7.0%, less than 6.5%, less than 6.0%, less than 5.5%, less than 5.0%, less than 4.5%, less than 4.0%, less than 3.5%, less than 3.0%, or less than 2.5% of the population of FcRn antagonist molecules. In some embodiments, the eighth subpopulation makes up about 7.5%, about 7.0%, about 6.5%, about 6.0%, about 5.5%, about 5.0%, about 4.5%, about 4.0%, about 3.5%, about 3.0%, or about 2.5% of the population of FcRn antagonist molecules. In some embodiments, the eighth subpopulation makes up 7.5%, 7.0%, 6.5%, 6.0%, 5.5%, 5.0%, 4.5%, 4.0%, 3.5%, 3.0%, or 2.5% of the population of FcRn antagonist molecules. In some embodiments, the eighth subpopulation makes up 2.5%-7.5%, 3.0%-5.0%, or 3.5%-4.5% of the population of FcRn antagonist molecules. In some embodiments, the eighth subpopulation makes up 2.9%-7.4% or 3.0%-6.3% of the population of FcRn antagonist molecules.

In some embodiments, the ninth subpopulation makes up less than 3.5%, less than 3.0%, less than 2.5%, less than 2.0%, less than 1.5%, less than 1%, or less than 0.5% of the population of FcRn antagonist molecules. In some embodiments, the ninth subpopulation makes up about 3.5%, about 3.0%, about 2.5%, about 2.0%, about 1.5%, about 1%, or about 0.5% of the population of FcRn antagonist molecules. In some embodiments, the ninth subpopulation makes up 3.5%, 3.0%, 2.5%, 2.0%, 1.5%, 1%, or 0.5% of the population of FcRn antagonist molecules. In some embodiments, the ninth subpopulation makes up 0.5%-3.5%, 1.5%-2.0%, or 1.0%-1.5% of the population of FcRn antagonist molecules. In some embodiments, the ninth subpopulation makes up 0.4%-3.2% or 0.5%-2.6% of the population of FcRn antagonist molecules.

In some embodiments, the tenth subpopulation makes up less than 2.0%, less than 1.5%, less than 1%, or less than 0.5% of the population of FcRn antagonist molecules. In some embodiments, the tenth subpopulation makes up about 2.0%, about 1.5%, about 1%, or about 0.5% of the population of FcRn antagonist molecules. In some embodiments, the tenth subpopulation makes up 2.0%, 1.5%, 1%, or 0.5% of the population of FcRn antagonist molecules. In some embodiments, the tenth subpopulation makes up 0.5%-2.0%, 0.5%-1.5%, or 1.0%-1.5% of the population of FcRn antagonist molecules.

In some embodiments, the eleventh subpopulation makes up less than 2.0%, less than 1.5%, less than 1%, or less than 0.5% of the population of FcRn antagonist molecules. In some embodiments, the eleventh subpopulation makes up about 2.0%, about 1.5%, about 1%, or about 0.5% of the population of FcRn antagonist molecules. In some embodiments, the eleventh subpopulation makes up 2.0%, 1.5%, 1%, or 0.5% of the population of FcRn antagonist molecules. In some embodiments, the eleventh subpopulation makes up 0.5%-2.0%, 0.5%-1.5%, or 1.0%-1.5% of the population of FcRn antagonist molecules.

In some embodiments, the FcRn antagonist molecules in the population comprise glycanation on one or both of their Fc domains. In some embodiments, the FcRn antagonist molecules in the population comprise glycanation at EU position 297 on one or both of their Fc domains. In some embodiments, the glycanation comprises an N-glycan. In some embodiments, the N-glycan comprises, a G0F N-glycan, G1F N-glycan, G2F N-glycan, or G0 N-glycan.

In some embodiments, the FcRn antagonist molecules comprise or consist of a variant Fc region comprising or consisting of a dimer of a first Fc domain and a second Fc domain, wherein one of the Fc domains is glycanated. In some embodiments, the glycanation comprises an N-glycan. In some embodiments, the N-glycan comprises, a G0F N-glycan, G1F N-glycan, G2F N-glycan, or G0 N-glycan.

In some embodiments, the FcRn antagonist molecules comprise or consist of a variant Fc region comprising or consisting of a dimer of a first Fc domain and a second Fc domain, wherein both of the Fc domains are glycanated. In some embodiments, the glycanation comprises an N-glycan. In some embodiments, the N-glycan comprises, a G0F N-glycan, G1F N-glycan, G2F N-glycan, or G0 N-glycan. In some embodiments, the Fc domains comprise a G0F N-glycan. In some embodiments, the Fc domains comprise a G1F N-glycan. In some embodiments, the Fc domains comprise a G2F N-glycan. In some embodiments, the Fc domains comprise a G0 N-glycan.

In some embodiments, the first Fc domain and the second Fc domain have different N-glycanations at EU position 297. In some embodiments, the first Fc domain comprises a GOF N-glycan, and the second Fc domain comprises a G1F N-glycan. In some embodiments, the first Fc domain comprises a G0F N-glycan, and the second Fc domain comprises a G2F N-glycan. In some embodiments, the first Fc domain comprises a G0F N-glycan, and the second Fc domain comprises a G0 N-glycan. In some embodiments, the first Fc domain comprises a G1F N-glycan, and the second Fc domain comprises a G2F N-glycan. In some embodiments, the first Fc domain comprises a G1F N-glycan, and the second Fc domain comprises a G2F+NANA N-glycan. In some embodiments, the first Fc domain comprises a G2F N-glycan, and the second Fc domain comprises a G2F+2×NANA N-glycan. In some embodiments, the first Fc domain comprises a G1F N-glycan, and the second Fc domain comprises a G0 N-glycan. In some embodiments, the first Fc domain comprises a G2F N-glycan, and the second Fc domain comprises a G0 N-glycan.

In some embodiments, the population comprises or consists of FcRn antagonist molecules, wherein at least 95%, at least 96%, at least 97%, at least 98%, or at least 99% of the population of Fc domains of the FcRn antagonist molecules comprise an N-glycan at EU position 297. In some embodiments, the population comprises or consists of FcRn antagonist molecules, wherein about 95%, about 96%, about 97%, about 98%, or about 99% of the population of Fc domains of the FcRn antagonist molecules comprise an N-glycan at EU position 297. In some embodiments, the population comprises or consists of FcRn antagonist molecules, wherein 95%, 96%, 97%, 98%, or 99% of the population of Fc domains of the FcRn antagonist molecules comprise an N-glycan at EU position 297. In some embodiments, the population comprises or consists of FcRn antagonist molecules, wherein 95%-99%, 96%-99%, or 97%-99% % of the population of Fc domains of the FcRn antagonist molecules comprise an N-glycan at EU position 297.

In some embodiments, the population comprises or consists of FcRn antagonist molecules, wherein at least 48%, at least 49%, at least 50%, at least 51%, at least 52%, at least 53%, at least 54%, at least 55%, at least 56%, at least 57%, at least 58%, at least 59%, at least 60%, at least 61%, at least 62%, at least 63%, at least 64%, at least 65%, at least 66%, or at least 67% of the population of Fc domains of the FcRn antagonist molecules comprise a G0F N-glycan at EU position 297. In some embodiments, the population comprises or consists of FcRn antagonist molecules, wherein about 48%, about 49%, about 50%, about 51%, about 52%, about 53%, about 54%, about 55%, about 56%, about 57%, about 58%, about 59%, about 60%, about 61%, about 62%, about 63%, about 64%, about 65%, about 66%, or about 67% of the population of Fc domains of the FcRn antagonist molecules comprise a G0F N-glycan at EU position 297. In some embodiments, the population comprises or consists of FcRn antagonist molecules, wherein 48%, 49%, 50%, 51%, 52%, 53%, 54%, 55%, 56%, 57%, 58%, 59%, 60%, 61%, 62%, 63%, 64%, 65%, 66%, or 67% of the population of Fc domains of the FcRn antagonist molecules comprise a GOF N-glycan at EU position 297. In some embodiments, the population comprises or consists of FcRn antagonist molecules, wherein 48%-67%, 51%-66%, or 50%-65% of the population of Fc domains of the FcRn antagonist molecules comprise a G0F N-glycan at EU position 297. In some embodiments, the population comprises or consists of FcRn antagonist molecules, wherein 53.8%-64.1% or 53.8%-63.1% of the population of Fc domains of the FcRn antagonist molecules comprise a G0F N-glycan at EU position 297.

In some embodiments, the population comprises or consists of FcRn antagonist molecules, wherein at least 21%, at least 22%, at least 23%, at least 24%, at least 25%, at least 26%, at least 27%, at least 28%, at least 29%, at least 30%, at least 31%, or at least 32% of the population of Fc domains of the FcRn antagonist molecules comprise a G1F N-glycan at EU position 297. In some embodiments, the population comprises or consists of FcRn antagonist molecules, wherein about 21%, about 22%, about 23%, about 24%, about 25%, about 26%, about 27%, about 28%, about 29%, or about 30%, about 31%, about 32% of the population of Fc domains of the FcRn antagonist molecules comprise a G1F N-glycan at EU position 297. In some embodiments, the population comprises or consists of FcRn antagonist molecules, wherein 21%, 22%, 23%, 24%, 25%, 26%, 27%, 28%, 29%, 30%, 31%, or 32% of the population of Fc domains of the FcRn antagonist molecules comprise a G1F N-glycan at EU position 297. In some embodiments, the population comprises or consists of FcRn antagonist molecules, wherein 21%-32%, 24%-31%, or 25%-30% of the population of Fc domains of the FcRn antagonist molecules comprise a G1F N-glycan at EU position 297 . . . . In some embodiments, the population comprises or consists of FcRn antagonist molecules, wherein 22.6%-28.3% or 23.5%-28.0% of the population of Fc domains of the FcRn antagonist molecules comprise a G1F N-glycan at EU position 297.

In some embodiments, the population comprises or consists of FcRn antagonist molecules, wherein at least 3%, at least 4%, at least 5%, at least 6%, at least 7%, at least 8%, at least 9%, at least 10%, at least 11%, or at least 12% of the population of Fc domains of the FcRn antagonist molecules comprise a G2F N-glycan at EU position 297. In some embodiments, the population comprises or consists of FcRn antagonist molecules, wherein about 3%, about 4%, about 5%, about 6%, about 7%, about 8%, about 9%, about 10%, about 11%, or about 12% of the population of Fc domains of the FcRn antagonist molecules comprise a G2F N-glycan at EU position 297. In some embodiments, the population comprises or consists of FcRn antagonist molecules, wherein 3%, 4%, 5%, 6%, 7%, 8%, 9%, 10%, 11%, or 12% of the population of Fc domains of the FcRn antagonist molecules comprise a G2F N-glycan at EU position 297. In some embodiments, the population comprises or consists of FcRn antagonist molecules, wherein 3%-12%, 5%-11%, or 7%-10% of the population of Fc domains of the FcRn antagonist molecules comprise a G2F N-glycan at EU position 297. In some embodiments, the population comprises or consists of FcRn antagonist molecules, wherein 5.6%-9.2% or 6.2%-9.2% of the population of Fc domains of the FcRn antagonist molecules comprise a G2F N-glycan at EU position 297.

In some embodiments, the population comprises or consists of FcRn antagonist molecules, wherein at least 1%, at least 2%, at least 3%, at least 4%, at least 5%, or at least 6% of the population of Fc domains of the FcRn antagonist molecules comprise a G0 N-glycan at EU position 297. In some embodiments, the population comprises or consists of FcRn antagonist molecules, wherein about 1%, about 2%, about 3%, about 4%, about 5%, or about 6% of the population of Fc domains of the FcRn antagonist molecules comprise a G0 N-glycan at EU position 297. In some embodiments, the population comprises or consists of FcRn antagonist molecules, wherein 1%, 2%, 3%, 4%, 5%, or 6% of the population of Fc domains of the FcRn antagonist molecules comprise a G0 N-glycan at EU position 297. In some embodiments, the population comprises or consists of FcRn antagonist molecules, wherein 1%-6%, 2%-5%, or 2%-4% of the population of Fc domains of the FcRn antagonist molecules comprise a G0 N-glycan at EU position 297. In some embodiments, the population comprises or consists of FcRn antagonist molecules, wherein 2.5%-4.5% or 2.5%-3.2% of the population of Fc domains of the FcRn antagonist molecules comprise a G0 N-glycan at EU position 297.

In some embodiments, the population comprises or consists of FcRn antagonist molecules, wherein at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, or at least 57% of the population of Fc domains of the FcRn antagonist molecules comprise galactose. In some embodiments, the population comprises or consists of FcRn antagonist molecules, wherein about 33%, about 34%, about 35%, about 36%, about 37%, about 38%, about 39%, about 40%, about 41%, about 42%, about 43%, about 44%, about 45%, about 46%, about 47%, about 48%, about 49%, about 50%, about 51%, about 52%, about 53%, about 54%, about 55%, about 56%, or about 57% of the population of Fc domains of the FcRn antagonist molecules comprise galactose. In some embodiments, the population comprises or consists of FcRn antagonist molecules, wherein 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, or 57% of the population of Fc domains of the FcRn antagonist molecules comprise a galactose. In some embodiments, the population comprises or consists of FcRn antagonist molecules, wherein 33%-57%, 34%-56%, or 35%-55% of the population of Fc domains of the FcRn antagonist molecules comprise galactose.

In some embodiments, the population comprises or consists of FcRn antagonist molecules, wherein at least 88%, at least 89%, at least 90%, at least 91%, at least 92%, at least 93%, at least 94%, at least 95%, at least 96%, at least 97%, at least 98%, or at least 99% of the population of Fc domains of the FcRn antagonist molecules comprise fucose. In some embodiments, the population comprises or consists of FcRn antagonist molecules, wherein about 88%, about 89%, about 90%, about 91%, about 92%, about 93%, about 94%, about 95%, about 96%, about 97%, about 98%, or about 99% of the population of Fc domains of the FcRn antagonist molecules comprise fucose. In some embodiments, the population comprises or consists of FcRn antagonist molecules, wherein 88%, 89%, 90%, 91%, 92%, 93%, 94%, 95%, 96%, 97%, 98%, or 99% of the population of Fc domains of the FcRn antagonist molecules comprise a fucose. In some embodiments, the population comprises or consists of FcRn antagonist molecules, wherein 33%-57%, 34%-56%, or 35%-55% of the population of Fc domains of the FcRn antagonist molecules comprise fucose.

In some embodiments, the population comprises or consists of FcRn antagonist molecules, wherein at most 0.3%, at most 0.4%, at most 0.5%, at most 0.6%, at most 0.7%, at most 0.8%, at most 0.9%, at most 1.0%, at most 1.1%, at most 1.2%, at most 1.3%, at most 1.4%, at most 1.5%, at most 1.6%, or at most 1.7% of the population of Fc domains of the FcRn antagonist molecules comprise sialic acid. In some embodiments, the population comprises or consists of FcRn antagonist molecules, wherein about 0.3%, about 0.4%, about 0.5%, about 0.6%, about 0.7%, about 0.8%, about 0.9%, about 1.0%, about 1.1%, about 1.2%, about 1.3%, about 1.4%, about 1.5%, about 1.6%, or about 1.7% of the population of Fc domains of the FcRn antagonist molecules comprise sialic acid. In some embodiments, the population comprises or consists of FcRn antagonist molecules, wherein 0.3%, 0.4%, 0.5%, 0.6%, 0.7%, 0.8%, 0.9%, 1.0%, 1.1%, 1.2%, 1.3%, 1.4%, 1.5%, 1.6%, or 1.7% of the population of Fc domains of the FcRn antagonist molecules comprise a sialic acid. In some embodiments, the population comprises or consists of FcRn antagonist molecules, wherein 0.3%-1.7%, 0.4%-1.6%, or 0.5%-1.5% of the population of Fc domains of the FcRn antagonist molecules comprise sialic acid.

In some embodiments, the population comprises or consists of FcRn antagonist molecules, wherein at least 38%, at least 39%, at least 40%, at least 41% at least 42%, at least 43%, at least 44%, at least 45%, at least 46%, at least 47%, at least 48%, at least 49%, at least 50%, at least 51%, at least 52%, at least 53%, at least 54%, at least 55%, at least 56%, or at least 57% of the FcRn antagonist molecules comprise a first Fc domain comprising a G0F N-glycan at EU position 297 and a second Fc domain comprising a G0F N-glycan at EU position 297. In some embodiments, the population comprises or consists of FcRn antagonist molecules, wherein about 38%, about 39%, about 40%, about 41% about 42%, about 43%, about 44%, about 45%, about 46%, about 47%, about 48%, about 49%, about 50%, about 51%, about 52%, about 53%, about 54%, about 55%, about 56%, or about 57% of the FcRn antagonist molecules comprise a first Fc domain comprising a G0F N-glycan at EU position 297 and a second Fc domain comprising a GOF N-glycan at EU position 297. In some embodiments, the population comprises or consists of FcRn antagonist molecules, wherein 38%, 39%, 40%, 41% 42%, 43%, 44%, 45%, 46%, 47%, 48%, 49%, 50%, 51%, 52%, 53%, 54%, 55%, 56%, or 57% of the FcRn antagonist molecules comprise a first Fc domain comprising a G0F N-glycan at EU position 297 and a second Fc domain comprising a G0F N-glycan at EU position 297. In some embodiments, the population comprises or consists of FcRn antagonist molecules, wherein 38%-57%, 39%-56%, or 40%-55% of the FcRn antagonist molecules comprise a first Fc domain comprising a G0F N-glycan at EU position 297 and a second Fc domain comprising a G0F N-glycan at EU position 297.

In some embodiments, the population comprises or consists of FcRn antagonist molecules, wherein at least 18%, at least 19%, at least 20%, at least 21% at least 22%, at least 23%, at least 24%, at least 25%, at least 26%, or at least 27% of the FcRn antagonist molecules comprise a first Fc domain comprising a G0F N-glycan at EU position 297 and a second Fc domain comprising a G1F N-glycan at EU position 297. In some embodiments, the population comprises or consists of FcRn antagonist molecules, wherein about 18%, about 19%, about 20%, about 21% about 22%, about 23%, about 24%, about 25%, about 26%, or about 27% of the FcRn antagonist molecules comprise a first Fc domain comprising a G0F N-glycan at EU position 297 and a second Fc domain comprising a G1F N-glycan at EU position 297. In some embodiments, the population comprises or consists of FcRn antagonist molecules, wherein 18%, 19%, 20%, 21% 22%, 23%, 24%, 25%, 26%, or 27% of the FcRn antagonist molecules comprise a first Fc domain comprising a G0F N-glycan at EU position 297 and a second Fc domain comprising a G1F N-glycan at EU position 297. In some embodiments, the population comprises or consists of FcRn antagonist molecules, wherein 18%-27%, 19%-28%, or 20%-25% of the FcRn antagonist molecules comprise a first Fc domain comprising a G0F N-glycan at EU position 297 and a second Fc domain comprising a G1F N-glycan at EU position 297.

In some embodiments, the population comprises or consists of FcRn antagonist molecules, wherein at least 8%, at least 9%, at least 10%, at least 11% at least 12%, at least 13%, at least 14%, at least 15%, at least 16%, or at least 17% of the FcRn antagonist molecules comprise a first Fc domain comprising a G1F N-glycan at EU position 297 and a second Fc domain comprising a G1F N-glycan at EU position 297. In some embodiments, the population comprises or consists of FcRn antagonist molecules, wherein about 8%, about 9%, about 10%, about 11% about 12%, about 13%, about 14%, about 15%, about 16%, or about 17% of the FcRn antagonist molecules comprise a first Fc domain comprising a G1F N-glycan at EU position 297 and a second Fc domain comprising a G1F N-glycan at EU position 297. In some embodiments, the population comprises or consists of FcRn antagonist molecules, wherein 8%, 9%, 10%, 11% 12%, 13%, 14%, 15%, 16%, or 17% of the FcRn antagonist molecules comprise a first Fc domain comprising a GIF N-glycan at EU position 297 and a second Fc domain comprising a G1F N-glycan at EU position 297. In some embodiments, the population comprises or consists of FcRn antagonist molecules, wherein 8%-17%, 9%-28%, or 10%-15% of the FcRn antagonist molecules comprise a first Fc domain comprising a G1F N-glycan at EU position 297 and a second Fc domain comprising a GIF N-glycan at EU position 297.

In some embodiments, the population comprises or consists of FcRn antagonist molecules, wherein at least 8%, at least 9%, at least 10%, at least 11% at least 12%, at least 13%, at least 14%, at least 15%, at least 16%, or at least 17% of the FcRn antagonist molecules comprise a first Fc domain comprising a G0F N-glycan at EU position 297 and a second Fc domain comprising a G2F N-glycan at EU position 297. In some embodiments, the population comprises or consists of FcRn antagonist molecules, wherein about 8%, about 9%, about 10%, about 11% about 12%, about 13%, about 14%, about 15%, about 16%, or about 17% of the FcRn antagonist molecules comprise a first Fc domain comprising a G0F N-glycan at EU position 297 and a second Fc domain comprising a G2F N-glycan at EU position 297. In some embodiments, the population comprises or consists of FcRn antagonist molecules, wherein 8%, 9%, 10%, 11% 12%, 13%, 14%, 15%, 16%, or 17% of the FcRn antagonist molecules comprise a first Fc domain comprising a GOF N-glycan at EU position 297 and a second Fc domain comprising a G2F N-glycan at EU position 297. In some embodiments, the population comprises or consists of FcRn antagonist molecules, wherein 8%-17%, 9%-28%, or 10%-15% of the FcRn antagonist molecules comprise a first Fc domain comprising a G0F N-glycan at EU position 297 and a second Fc domain comprising a G2F N-glycan at EU position 297.

In some embodiments, the population comprises or consists of FcRn antagonist molecules, wherein at least 3%, at least 4%, at least 5%, at least 6% at least 7%, at least 8%, at least 9%, at least 10%, at least 11%, or at least 12% of the FcRn antagonist molecules comprise a first Fc domain comprising a G1F N-glycan at EU position 297 and a second Fc domain comprising a G2F N-glycan at EU position 297. In some embodiments, the population comprises or consists of FcRn antagonist molecules, wherein about 3%, about 4%, about 5%, about 6% about 7%, about 8%, about 9%, about 10%, about 11%, or about 12% of the FcRn antagonist molecules comprise a first Fc domain comprising a G1F N-glycan at EU position 297 and a second Fc domain comprising a G2F N-glycan at EU position 297. In some embodiments, the population comprises or consists of FcRn antagonist molecules, wherein 3%, 4%, 5%, 6% 7%, 8%, 9%, 10%, 11%, or 12% of the FcRn antagonist molecules comprise a first Fc domain comprising a G1F N-glycan at EU position 297 and a second Fc domain comprising a G2F N-glycan at EU position 297. In some embodiments, the population comprises or consists of FcRn antagonist molecules, wherein 3%-12%, 4%-11%, or 5%-10% of the FcRn antagonist molecules comprise a first Fc domain comprising a G1F N-glycan at EU position 297 and a second Fc domain comprising a G2F N-glycan at EU position 297.

In some embodiments, the population comprises or consists of FcRn antagonist molecules, wherein at least 1%, at least 2%, at least 3%, at least 4% at least 5%, or at least 6% of the FcRn antagonist molecules comprise a first Fc domain comprising a G2F N-glycan at EU position 297 and a second Fc domain comprising a G2F N-glycan at EU position 297. In some embodiments, the population comprises or consists of FcRn antagonist molecules, wherein about 1%, about 2%, about 3%, about 4% about 5%, or about 6% of the FcRn antagonist molecules comprise a first Fc domain comprising a G2F N-glycan at EU position 297 and a second Fc domain comprising a G2F N-glycan at EU position 297. In some embodiments, the population comprises or consists of FcRn antagonist molecules, wherein 1%, 2%, 3%, 4% 5%, or 6% of the FcRn antagonist molecules comprise a first Fc domain comprising a G2F N-glycan at EU position 297 and a second Fc domain comprising a G2F N-glycan at EU position 297. In some embodiments, the population comprises or consists of FcRn antagonist molecules, wherein 1%-6%, 1%-4%, or 2%-4% of the FcRn antagonist molecules comprise a first Fc domain comprising a G2F N-glycan at EU position 297 and a second Fc domain comprising a G2F N-glycan at EU position 297.

In some embodiments, the population comprises or consists of FcRn antagonist molecules, wherein at least 2%, at least 3%, at least 4%, at least 5% at least 6%, at least 7%, or at least 8% of the FcRn antagonist molecules comprise a first Fc domain comprising a G0F N-glycan at EU position 297 and a second Fc domain comprising a G0 N-glycan at EU position 297. In some embodiments, the population comprises or consists of FcRn antagonist molecules, wherein about 2%, about 3%, about 4%, about 5% about 6%, about 7%, or about 8% of the FcRn antagonist molecules comprise a first Fc domain comprising a G0F N-glycan at EU position 297 and a second Fc domain comprising a G0 N-glycan at EU position 297. In some embodiments, the population comprises or consists of FcRn antagonist molecules, wherein 2%, 3%, 4%, 5% 6%, 7%, 8% of the FcRn antagonist molecules comprise a first Fc domain comprising a G0F N-glycan at EU position 297 and a second Fc domain comprising a G0 N-glycan at EU position 297. In some embodiments, the population comprises or consists of FcRn antagonist molecules, wherein 2%-8%, 3%-7%, or 4%-6% of the FcRn antagonist molecules comprise a first Fc domain comprising a G0F N-glycan at EU position 297 and a second Fc domain comprising a G0 N-glycan at EU position 297.

In some embodiments, the FcRn antagonist molecules lack an amino acid at EU position 441 of one or both Fc domains. In some embodiments, the FcRn antagonist molecules comprise glycine and lysine at EU positions 440 and 441, respectively. In some embodiments, the FcRn antagonist molecules lack amino acids at EU positions 440 and 441. In some embodiments, the FcRn antagonist molecules comprise amidated proline at EU position 439. In some embodiments, the FcRn antagonist molecules comprise amidated proline at EU position 439. In some embodiments, the FcRn antagonist molecules lack amino acids at EU positions 440 and 441 and comprise amidated proline at EU position 439.

In some embodiments, the population comprises or consists of FcRn antagonist molecules, wherein at least 83%, at least 84%, at least 85%, at least 86%, at least 87%, at least 88%, at least 89%, at least 90%, at least 91%, at least 92%, at least 93%, at least 94%, at least 95%, at least 96%, or at least 97% of the Fc domains in the population lack an amino acid at EU position 441. In some embodiments, the population comprises or consists of FcRn antagonist molecules, wherein about 83%, about 84%, about 85%, about 86%, about 87%, about 88%, about 89%, about 90%, about 91%, about 92%, about 93%, about 94%, about 95%, about 96%, or about 97% of the Fc domains in the population lack an amino acid at EU position 441. In some embodiments, the population comprises or consists of FcRn antagonist molecules, wherein 83%, 84%, 85%, 86%, 87%, 88%, 89%, 90%, 91%, 92%, 93%, 94%, 95%, 96%, or 97% of the Fc domains in the population lack an amino acid at EU position 441. In some embodiments, the population comprises or consists of FcRn antagonist molecules, wherein 83%-97%, 84%-94%, or 85%-95% of the Fc domains in the population lack an amino acid at EU position 441.

In some embodiments, the population comprises or consists of FcRn antagonist molecules, wherein less than 3%, less than 4%, less than 5%, less than 6%, less than 7%, less than 8%, less than 9%, less than 10%, less than 11%, less than 12%, less than 13%, less than 14%, less than 15%, less than 16%, or less than 17% of the Fc domains in the population have glycine and lysine at EU positions 440 and 441, respectively. In some embodiments, the population comprises or consists of FcRn antagonist molecules, wherein about 3%, about 4%, about 5%, about 6%, about 7%, about 8%, about 9%, about 10%, about 11%, about 12%, about 13%, about 14%, about 15%, about 16%, or about 17% of the Fc domains in the population have glycine and lysine at EU positions 440 and 441, respectively. In some embodiments, the population comprises or consists of FcRn antagonist molecules, wherein 3%, 4%, 5%, 6%, 7%, 8%, 9%, 10%, 11%, 12%, 13%, 14%, 15%, 16%, or 17% of the Fc domains in the population have glycine and lysine at EU positions 440 and 441, respectively. In some embodiments, the population comprises or consists of FcRn antagonist molecules, wherein 3%-17%, 4%-14%, or 5%-15% of the Fc domains in the population have glycine and lysine at EU positions 440 and 441, respectively.

In some embodiments, the population comprises or consists of FcRn antagonist molecules, wherein less than 0.5%, less than 1.0%, less than 1.5%, or less than 2% of the Fc domains in the population lack amino acids at EU positions 440 and 441 and comprise amidated proline at EU position 439. In some embodiments, the population comprises or consists of FcRn antagonist molecules, wherein about 0.5%, about 1.0%, about 1.5%, or about 2% of the Fc domains in the population lack amino acids at EU positions 440 and 441 and comprise amidated proline at EU position 439. In some embodiments, the population comprises or consists of FcRn antagonist molecules, wherein 0.5%, 1.0%, 1.5%, or 2% of the Fc domains in the population lack amino acids at EU positions 440 and 441 and comprise amidated proline at EU position 439. In some embodiments, the population comprises or consists of FcRn antagonist molecules, wherein 0.5%-2%, 0.5%-1.5%, or 0.5%-1.0% of the Fc domains in the population lack amino acids at EU positions 440 and 441 and comprise amidated proline at EU position 439.

In some embodiments, the FcRn antagonist molecules comprise aspartate, lysine, threonine, histidine, threonine, and cysteine, at EU positions 221, 222, 223, 224, 225, and 226, respectively. In some embodiments, the FcRn antagonist molecules lack an amino acid at EU positions 221, and comprise lysine, threonine, histidine, threonine, and cysteine at EU positions 222, 223, 224, 225, and 226, respectively. In some embodiments, the FcRn antagonist molecules lack amino acids at EU positions 221 and 222, and comprise threonine, histidine, threonine, and cysteine at EU positions 223, 224, 225, and 226, respectively. In some embodiments, the FcRn antagonist molecules lack amino acids at EU positions 221-224, and have threonine, and cysteine at EU positions 225, and 226, respectively. In some embodiments, the FcRn antagonist molecules lack amino acids at EU positions 221, 222, 223, 224, 225, and 226.

In some embodiments, the population comprises or consists of FcRn antagonist molecules, wherein at least 93%, at least 94%, at least 95%, at least 96%, at least 97%, at least 98%, or at least 99% of the Fc domains in the population have aspartate, lysine, threonine, histidine, threonine, and cysteine, at EU positions 221, 222, 223, 224, 225, and 226, respectively. In some embodiments, the population comprises or consists of FcRn antagonist molecules, wherein about 93%, about 94%, about 95%, about 96%, about 97%, about 98%, or about 99% of the Fc domains in the population have aspartate, lysine, threonine, histidine, threonine, and cysteine, at EU positions 221, 222, 223, 224, 225, and 226, respectively. In some embodiments, the population comprises or consists of FcRn antagonist molecules, wherein 93%, 94%, 95%, 96%, 97%, 98%, or 99% of the Fc domains in the population have aspartate, lysine, threonine, histidine, threonine, and cysteine, at EU positions 221, 222, 223, 224, 225, and 226, respectively. In some embodiments, the population comprises or consists of FcRn antagonist molecules, wherein 93%-99%, 94%-99%, or 95%-99% of the Fc domains in the population have aspartate, lysine, threonine, histidine, threonine, and cysteine, at EU positions 221, 222, 223, 224, 225, and 226, respectively.

In some embodiments, the population comprises or consists of FcRn antagonist molecules, wherein less than 0.5%, less than 1.0%, or less than 1.5% of the Fc domains in the population lack an amino acid at EU positions 221, and have lysine, threonine, histidine, threonine, and cysteine at EU positions 222, 223, 224, 225, and 226, respectively. In some embodiments, the population comprises or consists of FcRn antagonist molecules, wherein about 0.5%, about 1.0%, or about 1.5% of the Fc domains in the population lack an amino acid at EU positions 221, and have lysine, threonine, histidine, threonine, and cysteine at EU positions 222, 223, 224, 225, and 226, respectively. In some embodiments, the population comprises or consists of FcRn antagonist molecules, wherein 0.5%, 1.0%, or 1.5% of the Fc domains in the population lack an amino acid at EU positions 221, and have lysine, threonine, histidine, threonine, and cysteine at EU positions 222, 223, 224, 225, and 226, respectively. In some embodiments, the population comprises or consists of FcRn antagonist molecules, wherein 0.5%-1.0% of the Fc domains in the population lack an amino acid at EU positions 221, and have lysine, threonine, histidine, threonine, and cysteine at EU positions 222, 223, 224, 225, and 226, respectively.

In some embodiments, the population comprises or consists of FcRn antagonist molecules, wherein less than 0.5%, less than 1.0%, or less than 1.5% of the Fc domains in the population lack amino acids at EU positions 221 and 222, and have threonine, histidine, threonine, and cysteine at EU positions 223, 224, 225, and 226, respectively. In some embodiments, the population comprises or consists of FcRn antagonist molecules, wherein about 0.5%, about 1.0%, or about 1.5% of the Fc domains in the population lack amino acids at EU positions 221 and 222, and have threonine, histidine, threonine, and cysteine at EU positions 223, 224, 225, and 226, respectively. In some embodiments, the population comprises or consists of FcRn antagonist molecules, wherein 0.5%, 1.0%, or 1.5% of the Fc domains in the population lack amino acids at EU positions 221 and 222, and have threonine, histidine, threonine, and cysteine at EU positions 223, 224, 225, and 226, respectively. In some embodiments, the population comprises or consists of FcRn antagonist molecules, wherein 0.5%-1.0% of the Fc domains in the population lack amino acids at EU positions 221 and 222, and have threonine, histidine, threonine, and cysteine at EU positions 223, 224, 225, and 226, respectively.

In some embodiments, the population comprises or consists of FcRn antagonist molecules, wherein less than 0.5%, less than 1.0%, less than 1.5%, less than 2.5%, or less than 3.0% of the Fc domains in the population lack amino acids at EU positions 221-224, and have threonine, and cysteine at EU positions 225, and 226, respectively. In some embodiments, the population comprises or consists of FcRn antagonist molecules, wherein about 0.5%, about 1.0%, about 1.5%, about 2.5%, or about 3.0% of the Fc domains in the population lack amino acids at EU positions 221-224, and have threonine, and cysteine at EU positions 225, and 226, respectively. In some embodiments, the population comprises or consists of FcRn antagonist molecules, wherein 0.5%, 1.0%, 1.5%, 2.5%, or 3.0% of the Fc domains in the population lack amino acids at EU positions 221-224, and have threonine, and cysteine at EU positions 225, and 226, respectively. In some embodiments, the population comprises or consists of FcRn antagonist molecules, wherein 0.5%-1.0% of the Fc domains in the population lack amino acids at EU positions 221-224, and have threonine, and cysteine at EU positions 225, and 226, respectively.

In some embodiments, the population comprises or consists of FcRn antagonist molecules, wherein less than 0.5%, less than 1.0%, or less than 1.5% of the Fc domains in the population lack amino acids at EU positions 221, 222, 223, 224, 225, and 226. In some embodiments, the population comprises or consists of FcRn antagonist molecules, wherein about 0.5%, about 1.0%, or about 1.5% of the Fc domains in the population lack amino acids at EU positions 221, 222, 223, 224, 225, and 226. In some embodiments, the population comprises or consists of FcRn antagonist molecules, wherein 0.5%, 1.0%, or 1.5% of the Fc domains in the population lack amino acids at EU positions 221, 222, 223, 224, 225, and 226. In some embodiments, the population comprises or consists of FcRn antagonist molecules, wherein 0.5%-1.0% of the Fc domains in the population lack amino acids at EU positions 221, 222, 223, 224, 225, and 226.

In some embodiments, the FcRn antagonist molecules comprise isomerization of the aspartate at EU position 280 or 401. In some embodiments, the population comprises or consists of FcRn antagonist molecules, wherein less than 0.5%, less than 1.0%, or less than 1.5% of the Fc domains in the population comprise isomerization of the aspartate at EU position 280 or 401. In some embodiments, the population comprises or consists of FcRn antagonist molecules, wherein about 0.5%, about 1.0%, or about 1.5% of the Fc domains in the population comprise isomerization of the aspartate at EU position 280 or 401. In some embodiments, the population comprises or consists of FcRn antagonist molecules, wherein 0.5%, 1.0%, or 1.5% of the Fc domains in the population comprise isomerization of the aspartate at EU position 280 or 401. In some embodiments, the population comprises or consists of FcRn antagonist molecules, wherein 0.5%-1.0% of the Fc domains in the population comprise isomerization of the aspartate at EU position 280 or 401.

In some embodiments, the FcRn antagonist molecules comprise deamidation of the asparagine at EU position 384, 389, or 390. In some embodiments, the FcRn antagonist molecules comprise deamidation of the asparagine at EU position 315. In some embodiments, the FcRn antagonist molecules comprise deamidation of the asparagine at EU position 361. In some embodiments, the FcRn antagonist molecules comprise deamidation of the asparagine at EU position 276 or 286.

In some embodiments, the population comprises or consists of FcRn antagonist molecules, wherein less than 8%, less than 9%, less than 10%, less than 11%, or less than 12% of the Fc domains in the population comprise deamidation of the asparagine at EU position 384, 389, or 390. In some embodiments, the population comprises or consists of FcRn antagonist molecules, wherein about 8%, about 9%, about 10%, about 11%, or about 12% of the Fc domains in the population comprise deamidation of the asparagine at EU position 384, 389, or 390. In some embodiments, the population comprises or consists of FcRn antagonist molecules, wherein 8%, 9%, 10%, 11%, or 12% of the Fc domains in the population comprise deamidation of the asparagine at EU position 384, 389, or 390. In some embodiments, the population comprises or consists of FcRn antagonist molecules, wherein 8%-12%, 7%-11%, or 8%-10% of the Fc domains in the population comprise deamidation of the asparagine at EU position 384, 389, or 390.

In some embodiments, the population comprises or consists of FcRn antagonist molecules, wherein less than 1%, less than 2%, less than 3%, less than 4%, or less than 5% of the Fc domains in the population comprise deamidation of the asparagine at EU position 315. In some embodiments, the population comprises or consists of FcRn antagonist molecules, wherein about 1%, about 2%, about 3%, about 4%, or about 5% of the Fc domains in the population comprise deamidation of the asparagine at EU position 315. In some embodiments, the population comprises or consists of FcRn antagonist molecules, wherein 1%, 2%, 3%, 4%, or 5% of the Fc domains in the population comprise deamidation of the asparagine at EU position 315. In some embodiments, the population comprises or consists of FcRn antagonist molecules, wherein 1%-5%, 2%-4%, or 2%-3% of the Fc domains in the population comprise deamidation of the asparagine at EU position 315.

In some embodiments, the population comprises or consists of FcRn antagonist molecules, wherein less than 1%, less than 2%, less than 3%, less than 4%, or less than 5% of the Fc domains in the population comprise deamidation of the asparagine at EU position 361. In some embodiments, the population comprises or consists of FcRn antagonist molecules, wherein about 1%, about 2%, about 3%, about 4%, or about 5% of the Fc domains in the population comprise deamidation of the asparagine at EU position 361. In some embodiments, the population comprises or consists of FcRn antagonist molecules, wherein 1%, 2%, 3%, 4%, or 5% of the Fc domains in the population comprise deamidation of the asparagine at EU position 361. In some embodiments, the population comprises or consists of FcRn antagonist molecules, wherein 1%-5%, 2%-4%, or 2%-3% of the Fc domains in the population comprise deamidation of the asparagine at EU position 361.

In some embodiments, the population comprises or consists of FcRn antagonist molecules, wherein less than 0.5%, less than 1.0%, or less than 1.5% of the Fc domains in the population comprise deamidation of the asparagine at EU position 276 or 286. In some embodiments, the population comprises or consists of FcRn antagonist molecules, wherein about 0.5%, about 1.0%, or about 1.5% of the Fc domains in the population comprise deamidation of the asparagine at EU position 276 or 286. In some embodiments, the population comprises or consists of FcRn antagonist molecules, wherein 0.5%, 1.0%, or 1.5% of the Fc domains in the population comprise deamidation of the asparagine at EU position 276 or 286. In some embodiments, the population comprises or consists of FcRn antagonist molecules, wherein 0.5%-1.0% of the Fc domains in the population comprise deamidation of the asparagine at EU position 276 or 286.

In some embodiments, the FcRn antagonist molecules comprise oxidization of the methionine at EU position 428. In some embodiments, the FcRn antagonist molecules comprise oxidization of the tryptophan at EU position 277.

In some embodiments, the population comprises or consists of FcRn antagonist molecules, wherein less than 3%, less than 4%, less than 5%, less than 6%, or less than 7% of the Fc domains in the population comprise oxidization of the methionine at EU position 428. In some embodiments, the population comprises or consists of FcRn antagonist molecules, wherein about 3%, about 4%, about 5%, about 6%, or about 7% of the Fc domains in the population oxidization of the methionine at EU position 428. In some embodiments, the population comprises or consists of FcRn antagonist molecules, wherein 3%, 4%, 5%, 6%, or 7% of the Fc domains in the population oxidization of the methionine at EU position 428. In some embodiments, the population comprises or consists of FcRn antagonist molecules, wherein 3%-7%, 4%-6%, or 4%-5% of the Fc domains in the population oxidization of the methionine at EU position 428.

In some embodiments, the population comprises or consists of FcRn antagonist molecules, wherein less than 0.5%, less than 1.0%, or less than 1.5% of the Fc domains in the population comprise oxidization of the tryptophan at EU position 277. In some embodiments, the population comprises or consists of FcRn antagonist molecules, wherein about 0.5%, about 1.0%, or about 1.5% of the Fc domains in the population comprise oxidization of the tryptophan at EU position 277. In some embodiments, the population comprises or consists of FcRn antagonist molecules, wherein 0.5%, 1.0%, or 1.5% of the Fc domains in the population comprise oxidization of the tryptophan at EU position 277. In some embodiments, the population comprises or consists of FcRn antagonist molecules, wherein 0.5%-1.0% of the Fc domains in the population comprise oxidization of the tryptophan at EU position 277.

In some embodiments, the FcRn antagonist molecules comprise amidation of the proline at EU position 445.

In some embodiments, the population comprises or consists of FcRn antagonist molecules, wherein less than 0.5%, less than 1.0%, or less than 1.5% of the Fc domains in the population comprise amidation of the proline at EU position 445. In some embodiments, the population comprises or consists of FcRn antagonist molecules, wherein about 0.5%, about 1.0%, or about 1.5% of the Fc domains in the population comprise amidation of the proline at EU position 445. In some embodiments, the population comprises or consists of FcRn antagonist molecules, wherein 0.5%, 1.0%, or 1.5% of the Fc domains in the population comprise amidation of the proline at EU position 445. In some embodiments, the population comprises or consists of FcRn antagonist molecules, wherein 0.5%-1.0% of the Fc domains in the population comprise amidation of the proline at EU position 445.

### Polynucleotides, Vectors, and Methods of Production

The disclosure also provides polynucleotides encoding the FcRn antagonist molecules disclosed herein or fragments thereof. In some embodiments, a polynucleotide described herein is isolated or purified. As used herein, an “isolated” polynucleotide is one which is separated from other nucleic acid molecules e.g., those that are present in a natural source (e.g., in a mouse or a human) of the polynucleotide, or which is substantially free of other cellular material or culture medium when produced by recombinant techniques, or which is substantially free of chemical precursors or other chemicals when chemically synthesized. The language “substantially free” includes, without limitation, preparations of polynucleotide having less than about 15%, 10%, 5%, 2%, 1%, 0.5%, or 0.1% (in particular, less than about 10%) of other material, e.g., cellular material, culture medium, other nucleic acid molecules, chemical precursors and/or other chemicals.

In an aspect, provided herein are polynucleotides comprising a nucleotide sequence encoding an FcRn antagonist molecule described herein. In some embodiments, the polynucleotides comprise a nucleotide sequence that encodes an Fc domain comprising an amino acid sequence at least 70%, 75%, 80%, 85%, 90%, 95%, 96%, 97%, 98%, or 99% identical to the amino acid sequence of any one of SEQ ID NOs: 1-22. In some embodiments, the polynucleotides consist of a nucleotide sequence that encodes an Fc domain comprising an amino acid sequence at least 70%, 75%, 80%, 85%, 90%, 95%, 96%, 97%, 98%, or 99% identical to the amino acid sequence of any one of SEQ ID NOs: 1-22.

In some embodiments, the polynucleotides comprise a nucleotide sequence that encodes an Fc domain comprising the amino acid sequence of any one of SEQ ID NOs: 1-22. In some embodiments, the polynucleotides comprise a nucleotide sequence that encodes an Fc domain consisting of the amino acid sequence of any one of SEQ ID NOs: 1-22.

In some embodiments, the polynucleotides comprise nucleotide sequences that encode two or more Fc domains. In some embodiments, the polynucleotides comprise nucleotide sequences that encode two Fc domains. In some embodiments, the polynucleotides comprise a first nucleotide sequence that encodes a first Fc domain and a second nucleotide sequence that encodes a second Fc domain. In some embodiments, the first nucleotide sequence and the second nucleotide sequence are comprised in distinct nucleic acid molecules. In some embodiments, the first nucleotide sequence and the second nucleotide sequence are comprised in the same nucleic acid molecule.

In some embodiments, the first and second nucleotide sequence encode the same Fc domain. In some embodiments, both the first and second nucleotide sequence encode an Fc domain comprising an amino acid sequence at least 70%, 75%, 80%, 85%, 90%, 95%, 96%, 97%, 98%, or 99% identical to the amino acid sequence of any one of SEQ ID NOs: 5-20 and 22. In some embodiments, both the first and second nucleotide sequence encode an Fc domain consisting of an amino acid sequence at least 70%, 75%, 80%, 85%, 90%, 95%, 96%, 97%, 98%, or 99% identical to the amino acid sequence of any one of SEQ ID NOs: 5-20 and 22. In some embodiments, both the first and second nucleotide sequence encode an Fc domain comprising the amino acid sequence of any one of SEQ ID NOs: 5-20 and 22. In some embodiments, both the first and second nucleotide sequence encode an Fc domain consisting of the amino acid sequence of any one of SEQ ID NOs: 5-20 and 22.

In some embodiments, the first and second nucleotide sequence encode different Fc domains. In some embodiments, the first nucleotide sequence encodes an Fc domain comprising an amino acid sequence at least 70%, 75%, 80%, 85%, 90%, 95%, 96%, 97%, 98%, or 99% identical to the amino acid sequence of any one of SEQ ID NOs: 1-22 and the second nucleotide sequence encodes a different Fc domain comprising an amino acid sequence at least 70%, 75%, 80%, 85%, 90%, 95%, 96%, 97%, 98%, or 99% identical to the amino acid sequence of any one of SEQ ID NOs: 1-22. In some embodiments, the first nucleotide sequence encodes an Fc domain consisting of an amino acid sequence at least 70%, 75%, 80%, 85%, 90%, 95%, 96%, 97%, 98%, or 99% identical to the amino acid sequence of any one of SEQ ID NOs: 1-22 and the second nucleotide sequence encodes a different Fc domain consisting of an amino acid sequence at least 70%, 75%, 80%, 85%, 90%, 95%, 96%, 97%, 98%, or 99% identical to the amino acid sequence of any one of SEQ ID NOs: 1-22. In some embodiments, the first nucleotide sequence encodes an Fc domain comprising the amino acid sequence of any one of SEQ ID NOs: 1-22 and the second nucleotide sequence encodes a different Fc domain comprising the amino acid sequence of any one of SEQ ID NOs: 1-22. In some embodiments, the first nucleotide sequence encodes an Fc domain consisting of the amino acid sequence of any one of SEQ ID NOs: 1-22 and the second nucleotide sequence encodes a different Fc domain consisting of the amino acid sequence of any one of SEQ ID NOs: 1-22.

Also provided herein are polynucleotides encoding a polypeptide as provided above that are optimized, e.g., by codon/RNA optimization, replacement with heterologous signal sequences, and elimination of mRNA instability elements. Methods to generate optimized nucleic acids for recombinant expression by introducing codon changes and/or eliminating inhibitory regions in the mRNA can be carried out by adapting the optimization methods described in, e.g., U.S. Pat. Nos. 5,965,726; 6,174,666; 6,291,664; 6,414,132; and 6,794,498, accordingly, all of which are herein incorporated by reference in their entireties. For example, potential splice sites and instability elements (e.g., A/T or A/U rich elements) within the RNA can be mutated without altering the amino acids encoded by the nucleic acid sequences to increase stability of the RNA for recombinant expression. The alterations utilize the degeneracy of the genetic code, e.g., using an alternative codon for an identical amino acid. In an embodiment, it can be desirable to alter one or more codons to encode a conservative mutation, e.g., a similar amino acid with similar chemical structure and properties and/or function as the original amino acid.

The polynucleotides can be obtained, and the nucleotide sequence of the polynucleotides determined, by any method known in the art. Nucleotide sequences encoding proteins described herein, and modified versions of these antibodies can be determined using methods well known in the art, i.e., nucleotide codons known to encode particular amino acids are assembled in such a way to generate a nucleic acid that encodes the protein. Such a polynucleotide encoding the protein can be assembled from chemically synthesized oligonucleotides (e.g., as described in Kutmeier G et al., (1994) BioTechniques 17:242-6, herein incorporated by reference in its entirety), which, briefly, involves the synthesis of overlapping oligonucleotides containing portions of the sequence encoding the antibody, annealing, and ligating of those oligonucleotides, and then amplification of the ligated oligonucleotides by PCR.

Alternatively, a polynucleotide encoding a protein described herein can be generated from nucleic acid from a suitable source (e.g., a hybridoma) using methods well known in the art (e.g., PCR and other molecular cloning methods). For example, PCR amplification using synthetic primers hybridizable to the 3′ and 5′ ends of a known sequence can be performed using genomic DNA obtained from hybridoma cells producing the polypeptide of interest. Such PCR amplification methods can be used to obtain nucleic acids comprising the sequence encoding the polypeptide. The amplified nucleic acids can be cloned into vectors for expression in host cells and for further cloning.

If a clone containing a nucleic acid encoding a particular polypeptide is not available, but the sequence of the polypeptide is known, a nucleic acid encoding the polypeptide can be chemically synthesized or obtained from a suitable source (e.g., a cDNA library generated from, or nucleic acid, preferably poly A+RNA, isolated from any tissue or cells expressing the polypeptide described herein) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the polypeptide. Amplified nucleic acids generated by PCR can then be cloned into replicable cloning vectors using any method well known in the art.

DNA encoding proteins described herein can be readily isolated and sequenced using conventional procedures. Hybridoma cells can serve as a source of such DNA. Once isolated, the DNA can be placed into expression vectors, which are then transfected into host cells such as E. coli cells, simian COS cells, Chinese hamster ovary (CHO) cells (e.g., CHO cells from the CHO GS System™ (Lonza)), or myeloma cells that do not otherwise produce the proteins described herein.

Also provided are polynucleotides that hybridize under high stringency, intermediate or lower stringency hybridization conditions to polynucleotides that encode a protein described herein.

Hybridization conditions have been described in the art and are known to one of skill in the art. For example, hybridization under stringent conditions can involve hybridization to filter-bound DNA in 6× sodium chloride/sodium citrate (SSC) at about 45° C. followed by one or more washes in 0.2×SSC/0.1% SDS at about 50-65° C.; hybridization under highly stringent conditions can involve hybridization to filter-bound nucleic acid in 6×SSC at about 45° C. followed by one or more washes in 0.1×SSC/0.2% SDS at about 68° C. Hybridization under other stringent hybridization conditions is known to those of skill in the art and has been described, see, e.g., Ausubel F M et al., eds., (1989) Current Protocols in Molecular Biology, Vol. I, Green Publishing Associates, Inc. and John Wiley & Sons, Inc., New York at pages 6.3.1-6.3.6 and 2.10.3, which is herein incorporated by reference in its entirety.

In an aspect, provided herein are cells (e.g., host cells) expressing (e.g., recombinantly) a protein described herein, and related polynucleotides and expression vectors. Provided herein are vectors (e.g., expression vectors) comprising polynucleotides comprising nucleotide sequences encoding a protein described herein for recombinant expression in host cells, preferably in mammalian cells (e.g., CHO cells). Also provided herein are host cells comprising such vectors for recombinantly expressing proteins described herein. In an aspect, provided herein are methods for producing a protein described herein, comprising expressing the polypeptide from a host cell.

Recombinant expression of a protein described herein generally involves construction of an expression vector containing a polynucleotide that encodes the polypeptide. Once a polynucleotide encoding a polypeptide described herein has been obtained, the vector for the production of the polypeptide can be produced by recombinant DNA technology using techniques well known in the art. Thus, methods for preparing a protein by expressing a polynucleotide containing a polypeptide encoding nucleotide sequence are described herein. Methods which are well known to those skilled in the art can be used to construct expression vectors containing polypeptide coding sequences and appropriate transcriptional and translational control signals. These methods include, for example, in vitro recombinant DNA techniques, synthetic techniques, and in vivo genetic recombination. Also provided are replicable vectors comprising a nucleotide sequence encoding containing a polypeptide described herein, operably linked to a promoter. An expression vector can be transferred to a cell (e.g., host cell) by conventional techniques and the resulting cells can then be cultured by conventional techniques to produce a polypeptide described herein or a fragment thereof. Thus, provided herein are host cells containing a polynucleotide encoding containing a polypeptide described herein or fragments thereof, or a heavy or light chain thereof, or fragment thereof, or a single chain antibody described herein, operably linked to a promoter for expression of such sequences in the host cell.

In an embodiment, a host cell comprises a polynucleotide comprising one of the first nucleotide sequences and one of the second nucleotide sequences described above. In another embodiment, a host cell comprises a first polynucleotide comprising one of the first nucleotide sequences described above, and a second polynucleotide comprising one of the first nucleotide sequences described above. In another embodiment, a host cell comprises a first vector comprising one of the first nucleotide sequences and one of the second nucleotide sequences described above. In another embodiment, a host cell comprises a first vector comprising one of the first nucleotide sequences and one of the second nucleotide sequences described above, and a second vector comprising a second polynucleotide comprising one of the first nucleotide sequences described above.

In some embodiments, an Fc domain expressed by a first host cell is associated with an Fc domain expressed by a second host cell to form an FcRn antagonist molecule. In some embodiments, provided herein are populations of host cells comprising such first host cells and such second host cells.

In some embodiments, provided herein is a population of vectors comprising a first vector comprising a polynucleotide encoding an Fc domain, and a second vector comprising a polynucleotide encoding an Fc domain. In some embodiments, provided herein is a population of vectors comprising a first vector comprising a polynucleotide encoding an Fc domain, and a second vector comprising a polynucleotide encoding an Fc domain. In some embodiments, provided herein is a population of vectors comprising a first vector comprising a polynucleotide encoding an Fc domain and a polynucleotide encoding an Fc domain. In some embodiments, provided herein is a population of vectors comprising a first vector comprising a polynucleotide encoding two Fc domains.

A variety of host-expression vector systems can be utilized to express polypeptides described herein (see, e.g., U.S. Pat. No. 5,807,715, which is herein incorporated by reference in its entirety). Such host-expression systems represent vehicles by which the coding sequences of interest can be produced and subsequently purified, but also represent cells which can, when transformed or transfected with the appropriate nucleotide coding sequences, express a polypeptide described herein in situ. These include but are not limited to microorganisms such as bacteria (e.g., E. coli and B. subtilis) transformed with, e.g., recombinant bacteriophage DNA, plasmid DNA, or cosmid DNA expression vectors containing antibody coding sequences; yeast (e.g., Saccharomyces and Pichia) transformed with, e.g., recombinant yeast expression vectors containing antibody coding sequences; insect cell systems infected with, e.g., recombinant virus expression vectors (e.g., baculovirus) containing antibody coding sequences; plant cell systems (e.g., green algae such as Chlamydomonas reinhardtii) infected with, e.g., recombinant virus expression vectors (e.g., cauliflower mosaic virus, CaMV; tobacco mosaic virus, TMV) or transformed with, e.g., recombinant plasmid expression vectors (e.g., Ti plasmid) containing antibody coding sequences; or mammalian cell systems (e.g., COS (e.g., COS1 or COS), CHO, BHK, MDCK, HEK 293, NS0, PER.C6, VERO, CRL7O3O, HsS78Bst, HeLa, NIH 3T3, HEK-293T, HepG2, SP210, R1.1, B-W, L-M, BSC1, BSC40, YB/20, and BMT10 cells) harboring, e.g., recombinant expression constructs containing promoters derived from the genome of mammalian cells (e.g., metallothionein promoter) or from mammalian viruses (e.g., the adenovirus late promoter; the vaccinia virus 7.5K promoter). In an embodiment, cells for expressing antibodies described herein are Chinese hamster ovary (CHO) cells, for example CHO cells from the CHO GS System™ (Lonza). In an embodiment, the heavy chain and/or light chain of an antibody produced by a CHO cell may have an N-terminal glutamine or glutamate residue replaced by pyroglutamate. In an embodiment, cells for expressing polypeptides described herein are human cells, e.g., human cell lines. In an embodiment, a mammalian expression vector is pOptiVEC™ or pcDNA3.3. In an embodiment, bacterial cells such as Escherichia coli, or eukaryotic cells (e.g., mammalian cells), are used for the expression of a recombinant polypeptide. For example, mammalian cells such as CHO cells, in conjunction with a vector such as the major intermediate early gene promoter element from human cytomegalovirus, are an effective expression system for antibodies (Foecking M K & Hofstetter H, (1986) Gene 45:101-5; and Cockett M I et al., (1990) Biotechnology 8 (7): 662-7, each of which is herein incorporated by reference in its entirety). In an embodiment, polypeptides described herein are produced by CHO cells or NS0 cells. In an embodiment, the expression of nucleotide sequences encoding polypeptides described herein which comprise one, two, or three binding sites for human FcRn is regulated by a constitutive promoter, inducible promoter, or tissue specific promoter.

In bacterial systems, a number of expression vectors can be advantageously selected depending upon the use intended for the antibody molecule being expressed. For example, when a large quantity of such a polypeptide is to be produced, for the generation of pharmaceutical compositions of an antibody molecule, vectors which direct the expression of high levels of fusion protein products that are readily purified can be desirable. Such vectors include, but are not limited to, the E. coli expression vector pUR278 (Ruether U & Mueller-Hill B, (1983) EMBO J 2:1791-1794), in which the coding sequence can be ligated individually into the vector in frame with the lac Z coding region so that a fusion protein is produced; pIN vectors (Inouye S & Inouye M, (1985) Nuc Acids Res 13:3101-3109; Van Heeke G & Schuster S M, (1989) J Biol Chem 24:5503-5509); and the like, all of which are herein incorporated by reference in their entireties. For example, pGEX vectors can also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV), for example, can be used as a vector to express foreign genes. The virus grows in Spodoptera frugiperda cells. The coding sequence can be cloned individually into non-essential regions (for example the polyhedrin gene) of the virus and placed under control of an AcNPV promoter (for example the polyhedrin promoter).

In mammalian host cells, a number of viral-based expression systems can be utilized. In cases where an adenovirus is used as an expression vector, the coding sequence of interest can be ligated to an adenovirus transcription/translation control complex, e.g., the late promoter and tripartite leader sequence. This chimeric gene can then be inserted in the adenovirus genome by in vitro or in vivo recombination. Insertion in a non-essential region of the viral genome (e.g., region E1 or E3) will result in a recombinant virus that is viable and capable of expressing the molecule in infected hosts (see, e.g., Logan J & Shenk T, (1984) PNAS 81 (12): 3655-9, which is herein incorporated by reference in its entirety). Specific initiation signals can also be required for efficient translation of inserted coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression can be enhanced by the inclusion of appropriate transcription enhancer elements, transcription terminators, etc. (see, e.g., Bitter G et al., (1987) Methods Enzymol. 153:516-544, which is herein incorporated by reference in its entirety).

In addition, a host cell strain can be chosen which modulates the expression of the inserted sequences or modifies and processes the gene product in the specific fashion desired. Such modifications (e.g., glycosylation) and processing (e.g., cleavage) of protein products can be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product can be used. Such mammalian host cells include but are not limited to CHO, VERO, BHK, HeLa, MDCK, HEK 293, NIH 3T3, W138, BT483, Hs578T, HTB2, BT20, T47D, NS0 (a murine myeloma cell line that does not endogenously produce any immunoglobulin chains), CRL7O3O, COS (e.g., COSI or COS), PER.C6, VERO, HsS78Bst, HEK-293T, HepG2, SP210, R1.1, B-W, L-M, BSC1, BSC40, YB/20, BMT10, and HsS78Bst cells. In an embodiment, proteins described herein are produced in mammalian cells, such as CHO cells.

In an embodiment, a polypeptide described herein comprises a portion of an antibody with reduced fucose content or no fucose content. Such proteins can be produced using techniques known to one skilled in the art. For example, the proteins can be expressed in cells deficient or lacking the ability to fucosylate. In an example, cell lines with a knockout of both alleles of α1,6-fucosyltransferase can be used to produce antibodies with reduced fucose content. The Potelligent® system (Lonza) is an example of such a system that can be used to produce antibodies with reduced fucose content.

For long-term, high-yield production of recombinant proteins, stable expression cells can be generated. For example, cell lines which stably express a protein described herein can be engineered. In an embodiment, a cell provided herein stably expresses an FcRn antagonist molecule.

In certain aspects, rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA/polynucleotide, engineered cells can be allowed to grow for one to two days in an enriched media, and then are switched to a selective media. The selectable marker in the recombinant plasmid confers resistance to the selection and allows cells to stably integrate the plasmid into their chromosomes and grow to form foci, which in turn can be cloned and expanded into cell lines. This method can advantageously be used to engineer cell lines which express a polypeptide comprising one, two, or three binding sites for human FcRn described herein or a fragment thereof. Such engineered cell lines can be particularly useful in the screening and evaluation of compositions that interact directly or indirectly with the polypeptide.

A number of selection systems can be used, including but not limited to the herpes simplex virus thymidine kinase (Wigler M et al., (1977) Cell 11(1): 223-32); hypoxanthineguanine phosphoribosyltransferase (Szybalska E H & Szybalski W, (1962) PNAS 48(12): 2026-2034); and adenine phosphoribosyltransferase (Lowy I et al., (1980) Cell 22(3): 817-23) genes in tk-, hgprt- or aprt-cells, respectively, all of which are herein incorporated by reference in their entireties. Also, antimetabolite resistance can be used as the basis of selection for the following genes: dhfr, which confers resistance to methotrexate (Wigler M et al., (1980) PNAS 77(6): 3567-70; O'Hare K et al., (1981) PNAS 78:1527-31); gpt, which confers resistance to mycophenolic acid (Mulligan R C & Berg P, (1981) PNAS 78(4): 2072-6); neo, which confers resistance to the aminoglycoside G-418 (Wu G Y & Wu C H, (1991) Biotherapy 3:87-95; Tolstoshev P, (1993) Ann Rev Pharmacol Toxicol 32:573-596; Mulligan R C, (1993) Science 260:926-932; and Morgan R A & Anderson W F, (1993) Ann Rev Biochem 62:191-217; Nabel G J & Felgner P L, (1993) Trends Biotechnol 11(5): 211-5); and hygro, which confers resistance to hygromycin (Santerre R F et al., (1984) Gene 30(1-3): 147-56), all of which are herein incorporated by reference in their entireties. Methods commonly known in the art of recombinant DNA technology can be routinely applied to select the desired recombinant clone and such methods are described, for example, in Ausubel F M et al., (eds.), Current Protocols in Molecular Biology, John Wiley & Sons, NY (1993); Kriegler M, Gene Transfer and Expression, A Laboratory Manual, Stockton Press, NY (1990); and in Chapters 12 and 13, Dracopoli N C et al., (eds.), Current Protocols in Human Genetics, John Wiley & Sons, NY (1994); Colbère-Garapin F et al., (1981) J Mol Biol 150:1-14, all of which are herein incorporated by reference in their entireties.

The expression levels of a polypeptide can be increased by vector amplification (for a review, see, Bebbington C R & Hentschel C C G, The use of vectors based on gene amplification for the expression of cloned genes in mammalian cells in DNA cloning, p. 163-188. In DNA Cloning, Vol III, A Practical Approach. D. M. Glover (Ed.) (Academic Press, New York, 1987), which is herein incorporated by reference in its entirety). When a marker in the vector system is amplifiable, increase in the level of inhibitor present in culture of host cell will increase the number of copies of the marker gene. Since the amplified region is associated with the gene of interest, production of the polypeptide will also increase (Crouse G F et al., (1983) Mol Cell Biol 3:257-66, which is herein incorporated by reference in its entirety).

The host cell can be co-transfected with two or more expression vectors described herein. The two vectors can contain identical selectable markers which enable equal expression of heavy and light chain polypeptides. The host cells can be co-transfected with different amounts of the two or more expression vectors. For example, host cells can be transfected with any one of the following ratios of a first expression vector and a second expression vector: about 1:1, 1:2, 1:3, 1:4, 1:5, 1:6, 1:7, 1:8, 1:9, 1:10, 1:12, 1:15, 1:20, 1:25, 1:30, 1:35, 1:40, 1:45, or 1:50.

Alternatively, a single vector can be used which encodes, and is capable of expressing, both polypeptides. The coding sequences can comprise cDNA or genomic DNA. The expression vector can be monocistronic or multicistronic. A multicistronic nucleic acid construct can encode 2, 3, 4, 5, 6, 7, 8, 9, 10, or more genes/nucleotide sequences, or in the range of 2-5, 5-10, or 10-20 genes/nucleotide sequences. For example, a bicistronic nucleic acid construct can comprise, in the following order, a promoter, a first gene and a second gene. In such an expression vector, the transcription of both genes can be driven by the promoter, whereas the translation of the mRNA from the first gene can be by a cap-dependent scanning mechanism, and the translation of the mRNA from the second gene can be by a cap-independent mechanism, e.g., by an IRES.

Once a polypeptide described herein has been produced by recombinant expression, it can be purified by any method known in the art for purification of a protein, for example, by chromatography (e.g., ion exchange, affinity, particularly by affinity for the specific antigen after Protein A, and sizing column chromatography), centrifugation, differential solubility, or by any other standard technique for the purification of proteins. Further, the polypeptides described herein can be fused to heterologous polypeptide sequences described herein or otherwise known in the art to facilitate purification.

In an embodiment, a polypeptide described herein is isolated or purified. In an embodiment, an isolated polypeptide is one that is substantially free of other polypeptides with different antigenic specificities than the isolated polypeptide. For example, in certain embodiments, a preparation of a protein described herein is substantially free of cellular material and/or chemical precursors. The language “substantially free of cellular material” includes preparations of a polypeptide in which the polypeptide is separated from cellular components of the cells from which it is isolated or recombinantly produced. Thus, a polypeptide that is substantially free of cellular material includes preparations of polypeptide having less than about 30%, 20%, 10%, 5%, 2%, 1%, 0.5%, or 0.1% (by dry weight) of heterologous protein (also referred to herein as a “contaminating protein”) and/or variants of a polypeptide, for example, different post-translational modified forms of a polypeptide or other different versions of a polypeptide (e.g., polypeptide fragments). When the polypeptide is recombinantly produced, it is also generally substantially free of culture medium, i.e., culture medium represents less than about 20%, 10%, 2%, 1%, 0.5%, or 0.1% of the volume of the protein preparation. When the polypeptide is produced by chemical synthesis, it is generally substantially free of chemical precursors or other chemicals, i.e., it is separated from chemical precursors or other chemicals, which are involved in the synthesis of the protein. Accordingly, such preparations of the protein have less than about 30%, 20%, 10%, or 5% (by dry weight) of chemical precursors or compounds other than the protein of interest. In an embodiment, polypeptides described herein are isolated or purified.

A polypeptide described herein can be produced by any method known in the art for the synthesis of proteins, for example, by chemical synthesis or by recombinant expression techniques. The methods described herein employ, unless otherwise indicated, conventional techniques in molecular biology, microbiology, genetic analysis, recombinant DNA, organic chemistry, biochemistry, PCR, oligonucleotide synthesis and modification, nucleic acid hybridization, and related fields within the skill of the art. These techniques are described, for example, in the references cited herein and are fully explained in the literature. See, e.g., Maniatis T et al., (1982) Molecular Cloning: A Laboratory Manual, Cold Spring Harbor Laboratory Press; Sambrook J et al., (1989), Molecular Cloning: A Laboratory Manual, Second Edition, Cold Spring Harbor Laboratory Press; Sambrook J et al., (2001) Molecular Cloning: A Laboratory Manual, Cold Spring Harbor Laboratory Press, Cold Spring Harbor, NY; Ausubel F M et al., Current Protocols in Molecular Biology, John Wiley & Sons (1987 and annual updates); Current Protocols in Immunology, John Wiley & Sons (1987 and annual updates); Gait (ed.) (1984) Oligonucleotide Synthesis: A Practical Approach, IRL Press; Eckstein (ed.) (1991) Oligonucleotides and Analogues: A Practical Approach, IRL Press; Birren B et al., (eds.) (1999) Genome Analysis: A Laboratory Manual, Cold Spring Harbor Laboratory Press, all of which are herein incorporated by reference in their entireties.

In an embodiment, a polypeptide described herein is prepared, expressed, created, or isolated by any means that involves creation, e.g., via synthesis, genetic engineering of DNA sequences. In an embodiment, such a polypeptide comprises sequences (e.g., DNA sequences or amino acid sequences) that do not naturally exist within the antibody germline repertoire of an animal or mammal (e.g., human) in vivo.

### Pharmaceutical Compositions

In an aspect, the instant disclosure provides pharmaceutical compositions comprising an FcRn antagonist molecule as disclosed herein for use in methods of treating an antibody-mediated disorder (e.g., an autoantibody-mediated disorder). In general, these FcRn antagonist molecules inhibit the binding of Fc-containing agents (e.g., antibodies and immunoadhesins) to FcRn in vivo, which results in an increased rate of degradation of the Fc-containing agents and, concomitantly, a reduced serum level of these agents.

In some embodiments, FcRn antagonist molecules of the current disclosure have a predicted molecular weight ranging from about 50 kDa, to about 57 kDa. In some embodiments, the average molecular weight of non-aggregated FcRn antagonist molecules in the population is 50 kDa-57 kDa, 51 kDa-56 kDa, 52 kDa-55 kDa, 54 kDa-55 kDa, or 54.4 kDa-54.7 kDa. In some embodiments, the average molecular weight of non-aggregated FcRn antagonist molecules in the population is about 50 kDa, about 51 kDa, about 52 kDa, about 53 kDa, about 54 kDa, about 55 kDa, about 56 kDa, or about 57 kDa. In some embodiments, the average molecular weight of non-aggregated FcRn antagonist molecules in the population is 50 kDa, 51 kDa, 52 kDa, 53 kDa, 54 kDa, 55 kDa, 56 kDa, or 57 kDa. In some embodiments, the average molecular weight of non-aggregated FcRn antagonist molecules in the population is about 54.0 kDa, about 54.1 kDa, about 54.2 kDa, about 54.3 kDa, about 54.4 kDa, about 54.5 kDa, about 54.6 kDa, about 54.7 kDa, about 54.8 kDa, or about 54.9 kDa. In some embodiments, the average molecular weight of non-aggregated FcRn antagonist molecules in the population is 54.0 kDa, 54.1 kDa, 54.2 kDa, 54.3 kDa, 54.4 kDa, 54.5 kDa, 54.6 kDa, 54.7 kDa, 54.8 kDa, or 54.9 kDa.

In some embodiments, no more than 0.1%, no more than 0.2%, no more than 0.3%, no more than 0.4%, no more than 0.5%, no more than 0.6%, no more than 0.7%, no more than 0.8%, no more than 0.9%, or no more than 1.0% of the FcRn antagonist molecules in the population are aggregated. In some embodiments, about 0.1%, about 0.2%, about 0.3%, about 0.4%, about 0.5% about 0.6%, about 0.7%, about 0.8%, about 0.9%, or about 1.0% of the FcRn antagonist molecules in the population are aggregated. In some embodiments, 0.1%, 0.2%, 0.3%, 0.4%, 0.5% 0.6%, 0.7%, 0.8%, 0.9%, or 1.0% of the FcRn antagonist molecules in the population are aggregated. In some embodiments, 0.1%-1.0%, 0.3%-0.8%, 0.4%-0.6%, or 0.3%-0.5% of the FcRn antagonist molecules in the population are aggregated.

In some embodiments, at least 90%, at least 91%, at least 92%, at least 93%, at least 94%, at least 95%, at least 96%, at least 97%, at least 98%, or at least 99% of the dimers in the population of FcRn antagonist molecules are linked by at least one disulfide bond. In some embodiments, about 90%, about 91%, about 92%, about 93%, about 94%, about 95%, about 96%, about 97%, about 98%, or about 99% of the dimers in the population of FcRn antagonist molecules are linked by at least one disulfide bond. In some embodiments, 90%, 91%, 92%, 93%, 94%, 95%, 96%, 97%, 98%, or 99% of the dimers in the population of FcRn antagonist molecules are linked by at least one disulfide bond. In some embodiments, 90%-99%, 92%-97%, 94%-96%, or 93%-95% of the dimers in the population of FcRn antagonist molecules are linked by at least one disulfide bond.

In some embodiments, no more than 0.2%, no more than 0.4%, no more than 0.6%, no more than 0.8%, no more than 1.0%, no more than 1.2%, no more than 1.4%, no more than 1.6%, no more than 1.8%, or no more than 2.0% of the FcRn antagonist molecules in the population have free thiol groups. In some embodiments, about 0.2%, about 0.4%, about 0.6%, about 0.8%, about 1.0%, about 1.2%, about 1.4%, about 1.6%, about 1.8%, or about 2.0% of the FcRn antagonist molecules in the population have free thiol groups. In some embodiments, 0.2%, 0.4%, 0.6%, 0.8%, 1.0%, 1.2%, 1.4%, 1.6%, 1.8%, or 2.0% of the FcRn antagonist molecules in the population have free thiol groups. In some embodiments, 0.2%-2.0%, 0.6%-1.6%, 0.8%-1.2%, or 0.7%-1.0% of the FcRn antagonist molecules in the population have free thiol groups.

In some embodiments, the FcRn antagonist molecules are administered intravenously (IV) or subcutaneously (SC).

For IV administration, in certain embodiments, the FcRn antagonist molecules may be administered in a formulation comprising sodium phosphate, sodium chloride, L-arginine hydrochloride, and polysorbate 80. In certain embodiments, the FcRn antagonist molecules may be administered in a formulation comprising about 25 mM sodium phosphate, about 100 mM sodium chloride, and about 150 mM L-arginine hydrochloride (pH 6.7), with about 0.02% (w/v) polysorbate 80. In certain embodiments, the FcRn antagonist molecules may be administered in a formulation comprising 25 mM sodium phosphate, 100 mM sodium chloride, and 150 mM L-arginine hydrochloride (pH 6.7), with 0.02% (w/v) polysorbate 80. In certain embodiments, the FcRn antagonist molecules may be administered in a formulation comprising about 25 mM sodium phosphate, about 100 mM sodium chloride, and about 150 mM L-arginine hydrochloride (pH 6.7), with about 0.02% (w/v) polysorbate 80, via intravenous infusion in a total volume of about 250 mL over a period of about 2 hours. In certain embodiments, the FcRn antagonist molecules may be administered in a formulation comprising 25 mM sodium phosphate, 100 mM sodium chloride, and 150 mM L-arginine hydrochloride (pH 6.7), with 0.02% (w/v) polysorbate 80, via intravenous infusion in a total volume of 250 mL over a period of 2 hours. See, e.g., WO2019110823A1, which is incorporated by reference herein in its entirety.

In certain embodiments, the FcRn antagonist molecules may be administered in a formulation comprising an aqueous solution comprising about 25 mM sodium phosphate, about 100 mM sodium chloride, and about 150 mM L-arginine hydrochloride with a pH of about 6.7, with about 0.02% (w/v) polysorbate 80, diluted for intravenous infusion to a total volume of about 125 mL over a period of about 1 hour. In certain embodiments, the FcRn antagonist molecules may be administered in a formulation comprising an aqueous solution comprising 25 mM sodium phosphate, 100 mM sodium chloride, and 150 mM L-arginine hydrochloride with a pH of 6.7, with 0.02% (w/v) polysorbate 80, diluted for intravenous infusion to a total volume of 125 mL over a period of 1 hour.

In certain embodiments, the FcRn antagonist molecules may be administered in a formulation comprising an aqueous solution comprising about 4 mM sodium phosphate, about 146 mM sodium chloride, about 24 mM L-arginine, and about 0.0032% (w/v) polysorbate 80, with a pH of about 6.7. This formulation is administered via intravenous infusion in a total volume of about 125 mL over a period of about 1 hour. In certain embodiments, the FcRn antagonist molecules may be administered in a formulation comprising an aqueous solution comprising 4 mM sodium phosphate, 146 mM sodium chloride, 24 mM L-arginine, and 0.0032% (w/v) polysorbate 80, with a pH of 6.7. This formulation is administered via intravenous infusion in a total volume of 125 mL over a period of 1 hour.

In certain embodiments, the FcRn antagonist molecules may be administered via IV infusion and is provided in a sterile, colorless, clear concentrate solution at a concentration of about 20 mg/mL. In certain embodiments, the FcRn antagonist molecules may be administered via IV infusion and is provided in a sterile, colorless, clear concentrate solution at a concentration of 20 mg/mL.

In certain embodiments, FcRn antagonist molecules may be administered via IV infusion and is provided in a vial (e.g., a single-dose vial). In certain embodiments, a vial of FcRn antagonist molecules contains about 400 mg of FcRn antagonist molecules at a concentration of about 20 mg/mL. In certain embodiments, a vial of FcRn antagonist molecules contains 400 mg of FcRn antagonist molecules at a concentration of 20 mg/mL. In certain embodiments, each mL of solution in a vial of FcRn antagonist molecules contains about 31.6 mg L-arginine hydrochloride, about 0.2 mg polysorbate 80, about 5.8 mg sodium chloride, about 2.4 mg sodium phosphate dibasic anhydrous, about 1.1 mg sodium phosphate monobasic monohydrate, and water for injection, USP, at a pH of about 6.7. In certain embodiments, each mL of solution in a vial of FcRn antagonist molecules contains 31.6 mg L-arginine hydrochloride, 0.2 mg polysorbate 80, 5.8 mg sodium chloride, 2.4 mg sodium phosphate dibasic anhydrous, 1.1 mg sodium phosphate monobasic monohydrate, and water for injection, USP, at a pH of 6.7.

In certain embodiments, for patients weighing under 120 kg, the FcRn antagonist molecules may be administered at a dose of about 10 mg/kg as an IV infusion. In certain embodiments, for patients weighing under 120 kg, the FcRn antagonist molecules may be administered at a dose of about 10 mg/kg as an IV infusion over about one hour. In certain embodiments, for patients weighing under 120 kg, the FcRn antagonist molecules may be administered at a dose of about 10 mg/kg as an IV infusion over about one hour once weekly. In certain embodiments, for patients weighing under 120 kg, the FcRn antagonist molecules may be administered at a dose of about 10 mg/kg as an IV infusion over about one hour once weekly for about 4 weeks. In certain embodiments, for patients weighing under 120 kg, the FcRn antagonist molecules may be administered at a dose of 10 mg/kg as an IV infusion. In certain embodiments, for patients weighing under 120 kg, the FcRn antagonist molecules may be administered at a dose of 10 mg/kg as an IV infusion over one hour. In certain embodiments, for patients weighing under 120 kg, the FcRn antagonist molecules may be administered at a dose of 10 mg/kg as an IV infusion over one hour once weekly. In certain embodiments, for patients weighing under 120 kg, the FcRn antagonist molecules may be administered at a dose of 10 mg/kg as an IV infusion over one hour once weekly for 4 weeks. In certain embodiments, for patients weighing 120 kg or more, the FcRn antagonist molecules may be administered at a dose of about 1200 mg per IV infusion. In certain embodiments, for patients weighing 120 kg or more, the FcRn antagonist molecules may be administered at a dose of 1200 mg per IV infusion.

For SC administration, in certain embodiments, the FcRn antagonist molecules may be administered alone. Alternatively, for SC administration, in certain embodiments, the FcRn antagonist molecules may be administered co-formulated with hyaluronidase, for example, in particular, rHuPH20. The co-formulated material will allow SC dosing of larger volumes.

In some embodiments, the FcRn antagonist molecules may be administered in a formulation comprising an aqueous solution comprising about 20 mM L-histidine, about 100 mM sodium chloride, about 60 mM sucrose, about 10 mM L-methionine, and about 0.04% (w/v) polysorbate 20, wherein the composition has a pH of about 6.0. In some embodiments, the formulation comprises about 180 mg/mL the FcRn antagonist molecules. In some embodiments, the FcRn antagonist molecules may be administered in a formulation comprising an aqueous solution comprising 20 mM L-histidine, 100 mM sodium chloride, 60 mM sucrose, 10 mM L-methionine, and 0.04% (w/v) polysorbate 20, wherein the composition has a pH of 6.0. In some embodiments, the formulation comprises 180 mg/mL the FcRn antagonist molecules.

In some embodiments, the FcRn antagonist molecules may be administered in a formulation comprising an aqueous solution comprising about 20 mM L-histidine, about 50 mM L-arginine, about 100 mM sodium chloride, about 60 mM sucrose, about 10 mM L-methionine, and about 0.04% (w/v) polysorbate 80, wherein the composition has a pH of about 6.0. In some embodiments, the formulation comprises about 200 mg/mL the FcRn antagonist molecules. In some embodiments, the FcRn antagonist molecules may be administered in a formulation comprising an aqueous solution comprising 20 mM L-histidine, 50 mM L-arginine, 100 mM sodium chloride, 60 mM sucrose, 10 mM L-methionine, and 0.04% (w/v) polysorbate 80, wherein the composition has a pH of 6.0. In some embodiments, the formulation comprises 200 mg/mL the FcRn antagonist molecules.

The formulations disclosed herein include bulk drug compositions useful in the manufacture of pharmaceutical compositions (e.g., compositions that are suitable for administration to a subject or patient) which can be used in the preparation of unit dosage forms. In an embodiment, a composition of the invention is a pharmaceutical composition. Such compositions comprise a prophylactically or therapeutically effective amount of one or more prophylactic or therapeutic agents (e.g., an FcRn antagonist molecule) of the invention (or other prophylactic or therapeutic agent), and a pharmaceutically acceptable carrier.

In some embodiments the pharmaceutical compositions are formulated for administration to a subject via any suitable route of administration, including, but not limited to, intramuscular, intravenous, intradermal, intraperitoneal, subcutaneous, epidural, nasal, oral, rectal, topical, inhalation, buccal (e.g., sublingual), and transdermal administration. In an embodiment, the pharmaceutical compositions are formulated to be suitable for intravenous administration to a subject. In an embodiment, the pharmaceutical compositions are formulated to be suitable for subcutaneous administration to a subject.

### Methods of Treatment

In an embodiment, the FcRn antagonist molecule antagonizes FcRn binding to an antibody Fc region. The disclosure provides methods of reducing serum IgG in a subject comprising administering to the subject a therapeutically effective amount of an FcRn antagonist molecule according to the disclosure or a pharmaceutical composition comprising the same. In an embodiment, the level of serum IgG is decreased in the subject following administration of the FcRn antagonist molecule compared to a baseline level of serum IgG. In an embodiment, a total serum IgG reduction of about 60% compared to baseline serum IgG level is obtained. In an embodiment, a total serum IgG reduction of about 65%, about 70%, about 75%, or about 80% compared to baseline serum IgG level is obtained. In an embodiment, a total serum IgG reduction of about 65% compared to baseline serum IgG level is obtained. In an embodiment, a total serum IgG reduction of about 70% compared to baseline serum IgG level is obtained. In an embodiment, a total serum IgG reduction of about 75% compared to baseline serum IgG level is obtained. In an embodiment, a total serum IgG reduction of about 80% compared to baseline serum IgG level is obtained.

In an embodiment, the level of FcRn is not decreased in the subject following administration of the FcRn antagonist molecule compared to a baseline level of FcRn. In an embodiment, an FcRn reduction of less than about 1%, 2%, 3%, 4%, or 5% compared to baseline FcRn level is observed. In an embodiment, an FcRn reduction of less than about 10% compared to baseline FcRn level is observed.

The disclosure also provides methods for treating an antibody-mediated disorder (e.g., an autoantibody-mediated disorder) in a subject comprising administering to the subject a therapeutically effective amount of an FcRn antagonist molecule according to the disclosure or a pharmaceutical composition comprising the same.

In some embodiments, the antibody-mediated disorder is an autoimmune disease. In some embodiments, the autoimmune disease is selected from the group consisting of allogenic islet graft rejection, alopecia areata, ankylosing spondylitis, antiphospholipid syndrome, autoimmune Addison's disease, Alzheimer's disease, antineutrophil cytoplasmic autoantibodies (ANCA), autoimmune diseases of the adrenal gland, autoimmune hemolytic anemia, autoimmune hepatitis, autoimmune myocarditis, autoimmune neutropenia, autoimmune oophoritis and orchitis, immune thrombocytopenia (ITP or idiopathic thrombocytopenia purpura, idiopathic thrombocytopenia purpura, immune mediated thrombocytopenia, or primary immune thrombocytopenia), autoimmune urticaria, Behcet's disease, bullous pemphigoid (BP), cardiomyopathy, Castleman disease, celiac sprue-dermatitis, chronic fatigue immune disfunction syndrome, chronic inflammatory demyelinating polyneuropathy (CIDP), Churg-Strauss syndrome, cicatricial pemphigoid, CREST syndrome, cold agglutinin disease, Crohn's disease, dilated cardiomyopathy, discoid lupus, epidermolysis bullosa acquisita, essential mixed cryoglobulinemia, factor VIII deficiency, fibromyalgia-fibromyositis, glomerulonephritis, Graves' disease, Guillain-Barre syndrome, Goodpasture syndrome, graft-versus-host disease (GVHD), Hashimoto's thyroiditis, hemophilia A, idiopathic inflammatory myopathies (IIMs), idiopathic membranous neuropathy, idiopathic pulmonary fibrosis, IgA neuropathy, IgM polyneuropathies, immune-mediated necrotizing myopathy (IMNM), juvenile arthritis, Kawasaki's disease, lichen planus, lichen sclerosus, lupus erythematosus, lupus nephritis, Ménière's disease, mixed connective tissue disease, mucous membrane pemphigoid, multiple sclerosis, Type 1 diabetes mellitus, multifocal motor neuropathy (MMN), myasthenia gravis (MG), generalized myasthenia gravis (gMG), myositis, paraneoplastic bullous pemphigoid, pemphigoid gestationis, pemphigus vulgaris (PV), pemphigus foliaceus (PF), pernicious anemia, polyarteritis nodosa, polychrondritis, polyglandular syndromes, polymyalgia rheumatica, polymyositis, dermatomyositis (DM), necrotizing autoimmune myopathy (NAM), AntiSynthetase Syndrome (ASyS), primary agammaglobulinemia, primary biliary cirrhosis, psoriasis, psoriatic arthritis, relapsing polychondritis, Raynaud's phenomenon, Reiter's syndrome, rheumatoid arthritis, sarcoidosis, scleroderma, Sjögren's syndrome, solid organ transplant rejection, stiff-person syndrome, systemic lupus erythematosus, Takayasu's arteritis, toxic epidermal necrolysis (TEN), Stevens-Johnson syndrome (SJS), temporal arteritis/giant cell arteritis, thrombotic thrombocytopenia purpura, ulcerative colitis, uveitis, dermatitis herpetiformis vasculitis, anti-neutrophil cytoplasmic antibody-associated vasculitides, vitiligo, and Wegener's granulomatosis.

In an embodiment, at least one of the IgG subtypes is reduced in a subject following administration of the FcRn antagonist molecule. In an embodiment, at least one of the IgG subtypes is reduced in serum of a subject following administration of the FcRn antagonist molecule. In an embodiment, IgG1 is reduced. In an embodiment, IgG2 is reduced. In an embodiment, IgG3 is reduced. In an embodiment, IgG4 is reduced. In some embodiments, total serum IgG is reduced by at least 10%, at least 20%, at least 30%, at least 35%, at least 40%, at least 45%, at least 50%, at least 55%, at least 60%, at least 65%, or at least 70% in a subject following a single administration of the FcRn antagonist molecule.

In some embodiments, clearance of total serum IgG is increased in a subject following administration of the FcRn antagonist molecule. In some embodiments, clearance of total serum IgG in a subject following a single therapeutic administration of the FcRn antagonist molecule is comparable to the clearance of total serum IgG in a subject following a single therapeutic administration of efgartigimod. In some embodiments, clearance of total serum IgG in a subject following a single therapeutic administration of the FcRn antagonist molecule is similar or the same as the clearance of total serum IgG in a subject following a single therapeutic administration of efgartigimod.

In some embodiments, clearance of total serum IgG in a subject following a single administration of the FcRn antagonist molecule is comparable to the clearance of total serum IgG in a subject following a single administration of an equivalent amount of efgartigimod. In some embodiments, clearance of total serum IgG in a subject following a single administration of the FcRn antagonist molecule is similar or the same as the clearance of total serum IgG in a subject following a single administration of an equivalent amount of efgartigimod.

In an embodiment, the FcRn antagonist molecule is administered to the subject simultaneously or sequentially with an additional therapeutic agent. In an embodiment, the additional therapeutic agent is an anti-inflammatory agent. In an embodiment, the additional therapeutic agent is a corticosteroid. In an embodiment, the additional therapeutic agent is rituximab, daclizumab, basiliximab, muromonab-CD3, infliximab, adalimumab, omalizumab, efalizumab, natalizumab, tocilizumab, eculizumab, golimumab, canakinumab, ustekinumab, or belimumab. In an embodiment, the additional therapeutic agent is a leucocyte depleting agent.

In an embodiment, the additional therapeutic agent is a B-cell depleting agent. In an embodiment, the B-cell depleting agent is an antibody. In an embodiment, the B-cell depleting antibody is an antibody that specifically binds to CD10, CD19, CD20, CD21, CD22, CD23, CD24, CD37, CD53, CD70, CD72, CD74, CD75, CD77, CD79a, CD79b, CD80, CD81, CD82, CD83, CD84, CD85, or CD86.

In some embodiments, the FcRn antagonist molecule is administered intravenously. In some embodiments, the FcRn antagonist molecule is administered intravenously once weekly, once every two weeks, once every three weeks, once every four weeks, once monthly, or once every six weeks.

In some embodiments, the FcRn antagonist molecule is administered subcutaneously. In some embodiments, the FcRn antagonist molecule is administered subcutaneously once weekly, once every two weeks, once every three weeks, once every four weeks, once monthly, or once every six weeks.

### EXAMPLES

The following examples are offered by way of illustration, and not by way of limitation.

### Example 1: Identification of FcRn Antagonist Molecule Variants

A composition comprising a population of Fc antagonist molecules was characterized, and its constituents determined. Sample Batch 1 was used as an analytical reference standard. All analyses were performed on Batch 1 alongside a working reference standard, sample Batch 2, both of which were derived from the same drug substance batch, and on a reference standard sample Batch 3, used throughout clinical development. The batches were made using a vector designed to express SEQ ID NO:2 using a CHOKISV GS-KO cell line (Lonza Group Ltd.).

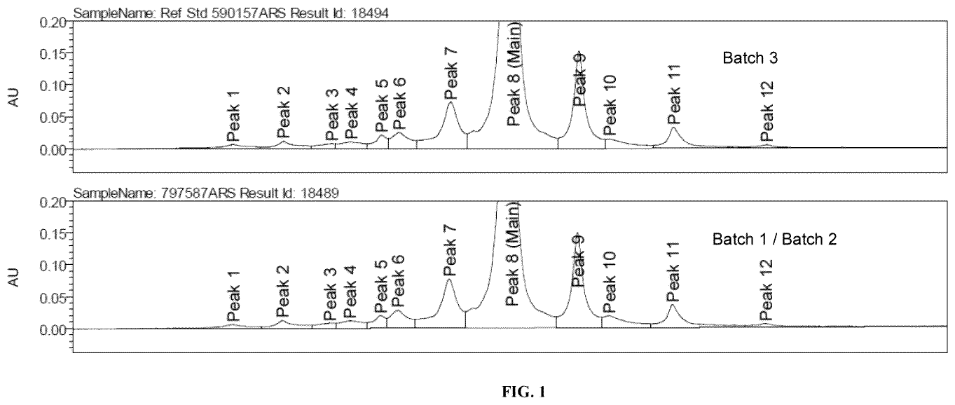

The charge heterogeneity of the reference samples was assessed using strong cation exchange high performance liquid chromatography (CEX HPLC). This assay was used to determine the charge heterogeneity profile of the sample to assess purity. Proteins present in the sample were separated and quantified according to their surface charge distribution, based on the interaction of charges on the surface of the protein with charged groups on the surface of the column. Proteins are positively charged in buffers with a pH value below their pl. Proteins were eluted from the column using a sodium chloride gradient (mobile phase B), with acidic species eluting first, followed by more basic species. Separated components passed through a UV detector cell and the absorbance was measured at a wavelength of 220 nm. The results are shown in Table 5 and FIG. 1.

Peaks of eluted species were classified as acidic or basic based on their elution time relative to the main peak in the CEX profile. Peaks eluting earlier than the main peak (peak 8) were identified as acidic species (peaks 1 to 7) and peaks eluting later (peaks 9 to 12) were identified as basic species. A total of twelve (12) charged species were identified by CEX for all three reference standards. The main isoform is present at a relative percentage area of approximately 65%. Total basic and acidic isoforms account for about 18% and 17% relative percentage area, respectively.

TABLE 5
CEX Results for FcRn Antagonist Reference Standards

| | | | | Batch 2 | |
|---|---|---|---|---|---|
| Peak | Isoform | Batch 3 | | Batch 1 | |
| Number | Type | RT | Area % | RT | Area % |
| 1 | Acidic | 21.0 | 1.4 | 20.8 | 1.3 |
| 2 | Acidic | 23.5 | 1.4 | 23.5 | 1.5 |
| 3 | Acidic | 26.2 | 0.8 | 26.1 | 0.8 |
| 4 | Acidic | 27.2 | 1.4 | 27.2 | 1.5 |
| 5 | Acidic | 28.9 | 1.5 | 28.9 | 1.3 |
| 6 | Acidic | 29.9 | 2.5 | 29.9 | 2.7 |
| 7 | Acidic | 32.7 | 7.9 | 32.7 | 8.1 |
| 8 | Main | 36.1 | 65.1 | 36.1 | 64.4 |
| 9 | Basic | 39.7 | 11.4 | 39.7 | 10.9 |
| 10 | Basic | 41.4 | 2.0 | 41.4 | 2.6 |
| 11 | Basic | 44.9 | 3.5 | 44.9 | 3.8 |
| 12 | Basic | 50.1 | 1.2 | 50.0 | 1.2 |

| | | |
|---|---|---|
| Report % acidic species | 16.8 | 17.2 |
| Report % main species | 65.1 | 64.4 |
| Report % basic species | 18.1 | 18.4 |

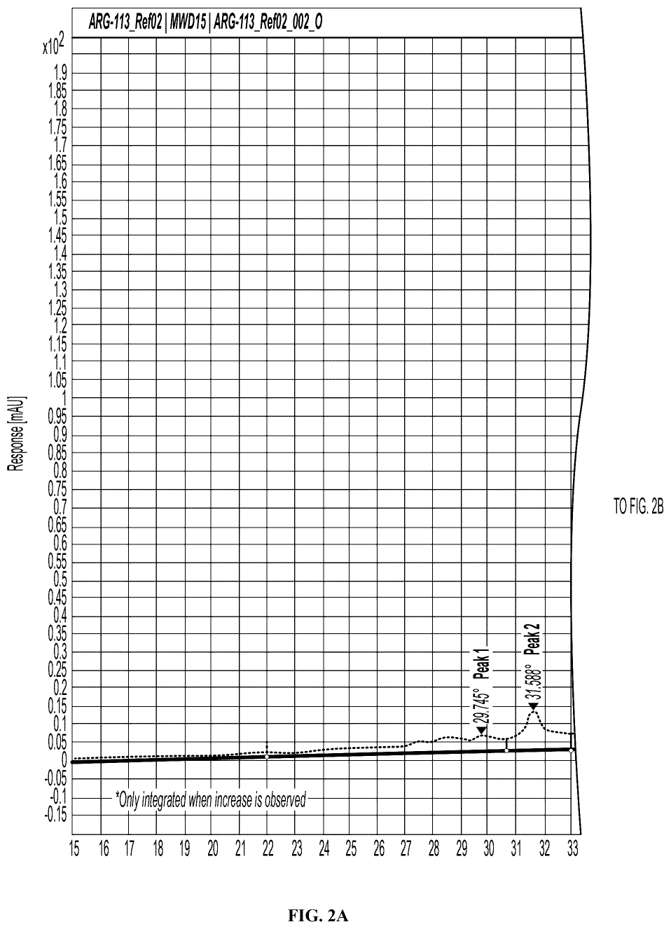

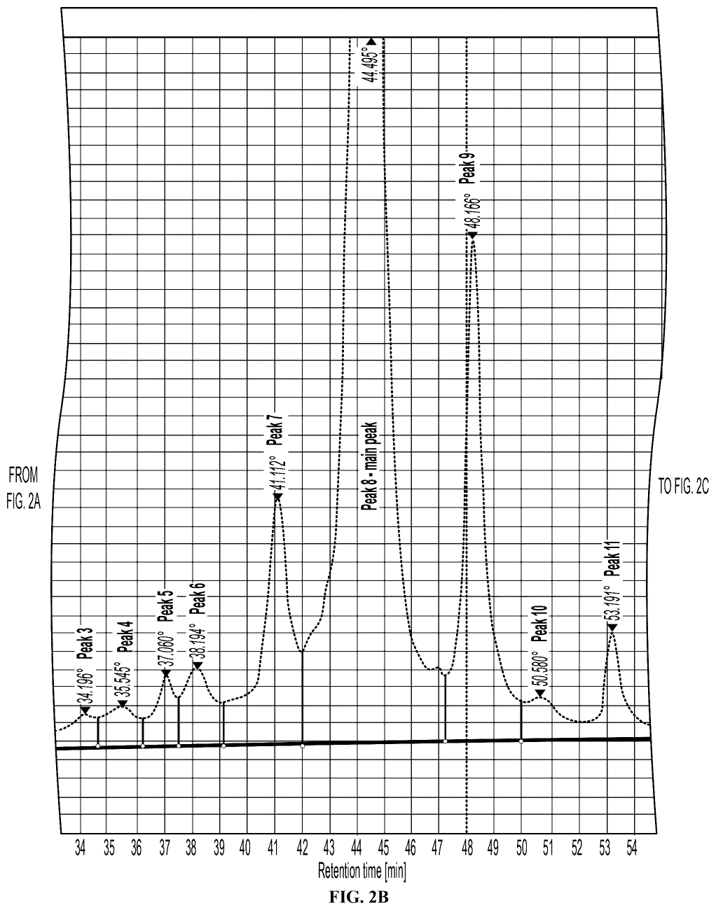

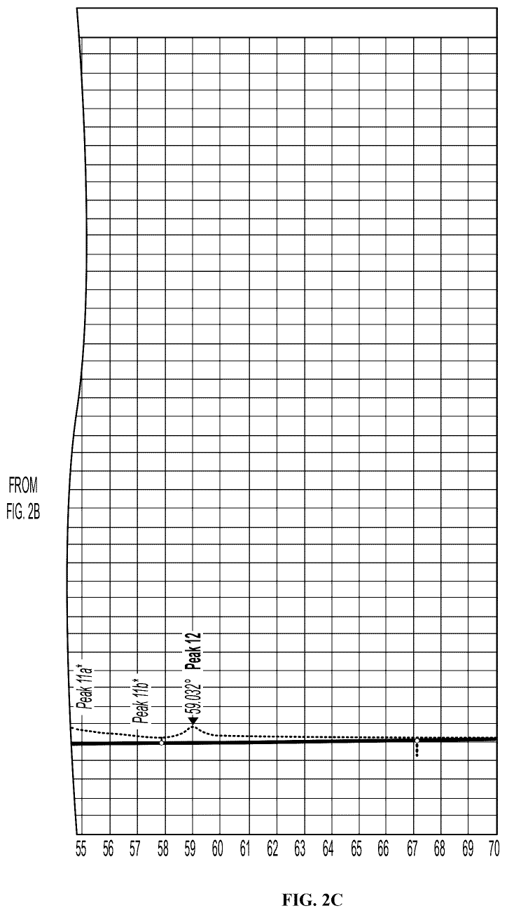

The identity of the different CEX isoform peaks was determined by reverse-phase liquid chromatography mass spectrometry (RPLC-MS) analysis of intact and trypsin digested isolated CEX fractions. The identity of the various peaks is presented in FIG. 2 and Table 6.

The identity of peak 9 (i.e., single lysine clipped variant) and peak 11 (i.e., lysine unclipped variant) was also confirmed by analyzing samples after treatment with carboxypeptidase B (CPB), resulting in complete lysine truncation.

TABLE 6
Identity of Charge Variants in CEX

| | Peak Identity | |
|---|---|---|
| | Long | Short |
| Peak No | Annotation | Annotation |
| Peak 1 | — | — |
| Peak 2 | — | — |
| Peak 3 | — | — |
| Peak 4 ¹ | Fc fragment, double C-terminal lysine | Fc/0K/−1DKTH |
| | clipped, single N-terminal loss of | |
| | DKTH variant | |
| Peak 5 | Fc fragment, double C-terminal lysine | Fc/0K/−1DK |
| | clipped, single N-terminal loss of | |
| | DK variant | |
| Peak 6 ^{1, 2} | Fc fragment, double C-terminal lysine | Fc/0K/+2deam |
| | clipped, double deamidated | |
| Peak 7 ^{1, 3} | Fc fragment, double C-terminal lysine | Fc/0K/+1deam |
| | clipped, single deamidated | |
| Peak 8 | Fc fragment, double C-terminal lysine | Fc/0K |
| main peak | clipped variant | |
| Peak 9 ⁴ | Fc fragment, single C-terminal lysine | Fc/1K |
| | clipped variant | |
| Peak 10 | Fc fragment, double C-terminal lysine | Fc/0K/+1ox |
| | clipped, single oxidized variant | |
| Peak 11 ⁴ | Fc fragment, non C-terminal lysine | Fc/2K |
| | clipped variant | |
| Peak 11a | Fc fragment, single C-terminal lysine | Fc/1K/+1ox |
| | clipped, single oxidized variant | |
| Peak 11b | Fc fragment, double C-terminal lysine | Fc/0K/+2ox |
| | clipped, double oxidized variant | |
| Peak 12 | Fc fragment, double C-terminal lysine | Fc/0K/−1D |
| | clipped, single N-terminal loss of | |
| | D variant | |

— = No identity could be determined as the abundance of this peak is too low for fraction collection.
¹ Traces of sialylated variants (Fc/0K + NANA) co-elute under peaks 4, 6 and 7.
² Fc fragment, double C-terminal lysine clipped, double C-terminal extended variant (Fc/0k/+2ext) elutes as well under peak 6, if present.
³ Fc fragment, double C-terminal lysine clipped, single C-terminal extended variant (Fc/0k/+1ext) elutes as well under peak 7, if present.
⁴ Since carboxypeptidase treatment did not result in a complete decrease of Peak 9 and Peak 11, an unknown variant is likely co-eluting at this retention time.

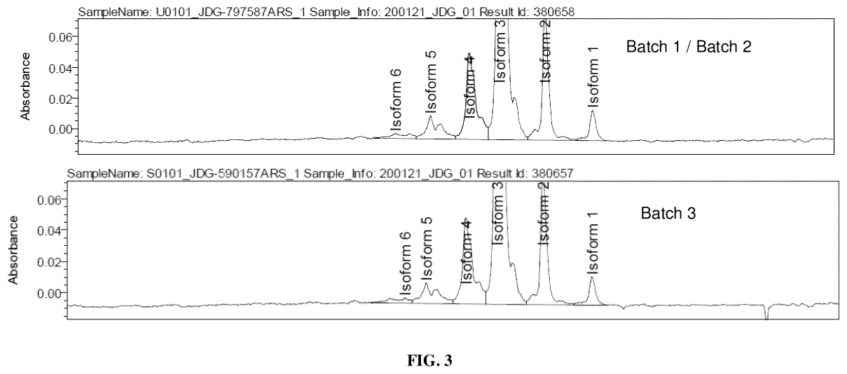

The charge heterogeneity of the reference samples was also evaluated by imaged capillary isoelectric focusing (icIEF). The results are shown in Table 7 and FIG. 3.

The peaks were numbered based on their measured isoelectric point (pI) relative to the major profile peak. Peaks displaying a pI at a pH lower than that of the major peak were identified as acidic species, and peaks displaying a pI at a higher pH were identified as basic species.

Six isoforms were detected across a pI range of pI 6.7 to 7.6. The main isoform was detected with a pI of approximately 7.2 and a relative percentage area of 67%. Total basic isoforms accounted for 15% relative percentage area and total acidic isoforms accounted for 18% relative percentage area.

TABLE 7
Results for icIEF Testing of Reference Standards

| | | |
|---|---|---|
| Isoform | Batch 3 | Batch 2/Batch 1 |

| Isoform | Type | pI | Relative % | pI | Relative % |
|---|---|---|---|---|---|
| 1 | Basic | 7.6 | 2.8 | 7.6 | 2.9 |
| 2 | Basic | 7.4 | 12.4 | 7.4 | 12.3 |
| 3 | Main | 7.2 | 66.5 | 7.2 | 66.7 |
| 4 | Acidic | 7.0 | 12.3 | 7.0 | 12.3 |
| 5 | Acidic | 6.8 | 4.5 | 6.8 | 4.3 |
| 6 | Acidic | 6.7 | 1.5 (<LOQ) | 6.7 | 1.4 (<LOQ) |

| | | |
|---|---|---|
| Total basic % | 15.2 | 15.2 |
| Total main % | 66.5 | 66.7 |
| Total acidic % | 18.4 | 18.1 |

Since fractionation of separated charge variants is more challenging for icIEF compared to CEX, a different approach was used to characterize the charge variants resolved by icIEF. The isolated and identified CEX fractions described above, were subjected to icIEF and, based on the electropherogram, identification of the isoforms was achieved. The identity of the different isoforms is presented in Table 8.

By subjecting the CEX fraction of peak 6 to icIEF, it can be concluded that the double deamidated variant and the double extended variant were eluting on icIEF as isoform 5. Earlier icIEF spiking studies with pure recombinantly produced double C-terminal extended variant indicated as well that double extended variant elutes as isoform 5 on icIEF.

CEX fractions of peak 7 eluted on icIEF as isoform 4, thus isoform 4 was identified as co-elution of the single deamidated and single extended variant.

The main variant on CEX, peak 8, identified as double lysine clipped Fc, eluted on icIEF as main isoform 3.

Isoform 2 on icIEF was identified as a single lysine clipped variant. This was confirmed by icIEF of CEX peak 9.

Isoform 1 was identified as non-lysine clipped variant by icIEF of CEX peak 11. The identity of the single lysine clipped variant and non-lysine clipped variant was also confirmed by analyzing samples after treatment with carboxypeptidase (CPB), resulting in complete lysine truncation. After treatment of samples with CPB, a decrease of isoform 1 and 2 was observed, and an unknown variant was observed eluting at the same retention time, similar to the observation for CEX.

Compared to the CEX profile, the oxidized members of the population of Fc antagonist molecules were not separated by icIEF. This was confirmed by analyzing the CEX fractions of peak 10 (single oxidation on the double lysine clipped variant) and peak 11b (double oxidation on the double lysine clipped variant), which both elute on icIEF as isoform 3 (previously confirmed as double lysine clipped variant) and by analyzing the CEX fractions of peak 11a (single lysine clipped and single oxidized variant), eluting on icIEF as isoform 2 (previously confirmed as single lysine clipped variant). This was related to the mechanism of separation. On icIEF the charge variants were separated based on apparent pI. On CEX, the charge variants were separated on surface charge. For the Fc antagonist molecules, the oxidation has an influence on the surface charge, resulting in a resolved peak on CEX.

TABLE 8
Identity of Charge Variants by icIEF
Based on Analysis of CEX Fractions

| | | Elution on |
|---|---|---|
| CEX Characterization results | | icIEF |
| Peak No | Long annotation | Peak No |
| Peak 1 | — | — |
| Peak 2 | — | — |
| Peak 3 | — | — |
| Peak 4 | Fc fragment, double C-terminal lysine clipped, | — |
| | single N-terminal loss of DKTH variant | |
| Peak 5 | Fc fragment, double C-terminal lysine clipped, | — |
| | single N-terminal loss of DK variant | |
| Peak 6 ¹ | Fc fragment, double C-terminal lysine clipped, | Isoform 5 |
| | double deamidated | |
| Peak 7 ² | Fc fragment, double C-terminal lysine clipped, | Isoform 4 |
| | single deamidated | |
| Peak 8 - | Fc fragment, double C-terminal lysine clipped | Isoform 3 |
| main peak | variant | |
| Peak 9 | Fc fragment, single C-terminal lysine clipped | Isoform 2 |
| | variant | |
| Peak 10 | Fc fragment, double C-terminal lysine clipped, | Isoform 3 |
| | single oxidized variant | |
| Peak 11 | Fc fragment, non C-terminal lysine clipped | Isoform 1 |
| | variant | |
| Peak 11a | Fc fragment, single C-terminal lysine clipped, | Isoform 2 |
| | single oxidized variant | |
| Peak 11b | Fc fragment, double C-terminal lysine clipped, | Isoform 3 |
| | double oxidized variant | |
| Peak 12 | Fc fragment, double C-terminal lysine clipped, | Isoform 2 |
| | single N-terminal loss of D variant | |

— = No identity could be determined as the abundance of this peak is too low for fraction collection and analysis on icIEF.
¹ Fc fragment, double C-terminal lysine clipped, double C-terminal extended variant (Fc/0k/+2ext) elutes as well under CEX peak 6, if present.
² Fc fragment, double C-terminal lysine clipped, single C-terminal extended variant (Fc/0k/+1ext) elutes as well under CEX peak 7, if present.

### Example 2: Identification of Prevalence of FcRn Antagonist Molecule Variants

Prevalence of N-terminal truncation was detected by RPLC-MS of intact and N-deglycosylated and reduced protein, and by reduced peptide mapping (tryptic and chymotryptic digest) and quantified by CEX HPLC. Relative % peak areas for peak 4 (N-terminal loss of DKTH), peak 5 (N-terminal loss of DK), and peak 12 (N-terminal loss of D) are shown in Table 9.

Comparable low levels of N-terminal truncation have been observed at release by CEX, as demonstrated in FIG. 1 with data obtained from reference standard samples and process qualification batches.

TABLE 9
Relative Percentages of N-terminal Truncation Determined by CEX

| N- | | | | | | | | | |
|---|---|---|---|---|---|---|---|---|---|
| Terminal | | Relative % Peak Area | | | | | | | |
| Truncated | CEX Peak | 590157 | PRS | PPQ1 | PPQ2 | PPQ3 | PPQ4 | PPQ5 | PPQ6 |
| Variant | Number | ARS | 797587 | Batch A | Batch B | Batch C | Batch D | Batch E | Batch F |
| Fc/0K/−DKTH | Peak 4 | 1.3 | 1.3 | 1.3 | 1.6 | 1.5 | 1.3 | 1.4 | 1.3 |
| Fc/0K/−1DK | Peak 5 | 1.4 | 1.3 | 1.8 | 1.8 | 1.8 | 1.3 | 1.6 | 1.5 |
| Intact N- | Peak 8 | 65.2 | 65.1 | 65.4 | 63.8 | 63.8 | 63.8 | 63.8 | 64.8 |
| terminus | (main peak) | | | | | | | | |
| Fc/0K/−1D | Peak 12 | 1.2 | 1.0 | 1.3 | 1.2 | 1.4 | 1.0 | 1.2 | 1.1 |

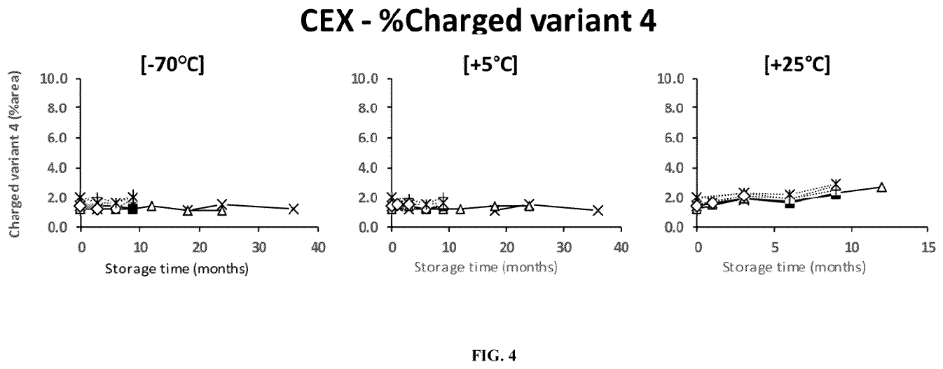

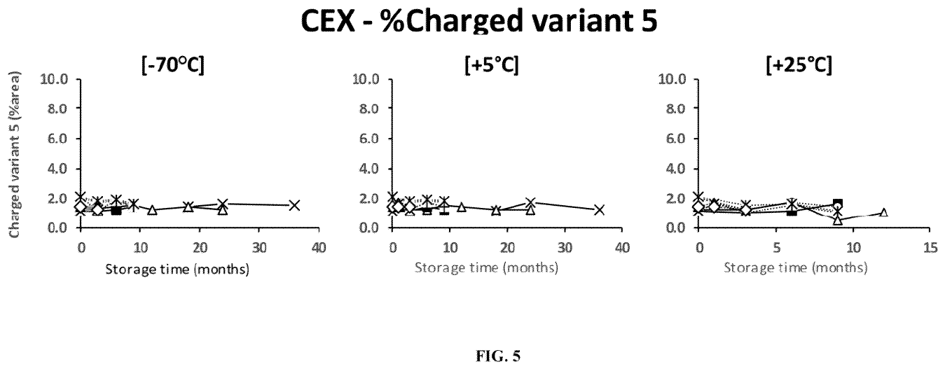

Further, during long term stability studies, no changes for peak 4 and 5 were observed at −70° C., +5° C. and +25° C., as demonstrated in FIG. 4 and FIG. 5, respectively.

N-terminal truncated variants were concluded to be quality attributes that contribute to product heterogeneity.

C-terminal lysine clipping can be detected by RPLC-MS of intact and N-deglycosylated and reduced protein and by reduced peptide mapping, and can be quantified by CEX HPLC and by icIEF. In the CEX profile, the main peak 8 and basic peaks 9 and 11 corresponded, respectively, with the double lysine clipped, the single lysine clipped, and the lysine unclipped variants (see Table 6). In the icIEF profile, the same isoforms appear as the main isoform (double lysine clipped), basic isoform 2 (single lysine clipped) and basic isoform 1 (lysine unclipped) (see Table 8).

Levels of C-terminal lysine clipped variants determined by CEX and icIEF were consistent across both methods between reference standard samples and process qualification batches, as demonstrated in Table 10 and Table 11.

TABLE 10
Relative Percentages of C-Terminal Lysine Clipped Variants Determined by CEX

| | | Relative % peak area | | | | | | | |
|---|---|---|---|---|---|---|---|---|---|
| C-Terminal | CEX Peak | 590157 | PRS | PPQ1 | PPQ2 | PPQ3 | PPQ4 | PPQ5 | PPQ6 |
| Variants | Number | ARS | 797587 | Batch A | Batch B | Batch C | Batch D | Batch E | Batch F |
| Double | Peak 8 | 65.2 | 65.1 | 65.4 | 63.8 | 63.8 | 63.8 | 63.8 | 64.8 |
| lysine | (main | | | | | | | | |
| clipped | peak) | | | | | | | | |
| variant (0K) | | | | | | | | | |
| Single lysine | Basic | 11.6 | 11.0 | 10.1 | 10.5 | 9.7 | 12.8 | 11.2 | 11.8 |
| clipped | Peak 9 | | | | | | | | |
| variant (1K) | | | | | | | | | |
| Double | Basic | 3.7 | 3.9 | 3.6 | 3.8 | 3.6 | 4.3 | 3.8 | 3.8 |
| lysine | Peak 11 | | | | | | | | |
| variant (0K) | | | | | | | | | |

TABLE 11
Relative Percentages of C-Terminal Lysine Clipped Variants by Determined icIEF

| | | Relative % peak area | | | | | | | |
|---|---|---|---|---|---|---|---|---|---|
| C-Terminal | icIEF Peak | 590157 | PRS | PPQ1 | PPQ2 | PPQ3 | PPQ4 | PPQ5 | PPQ6 |
| Variants | Number | ARS | 797587 | Batch A | Batch B | Batch C | Batch D | Batch E | Batch F |
| Double | Isoform 3 | 64.6 | 64.1 | 65.7 | 63.8 | 63.3 | 62.6 | 63.9 | 64.4 |
| lysine | (main | | | | | | | | |
| clipped | isoform) | | | | | | | | |
| variant (0K) | | | | | | | | | |
| Single | Basic | 12.1 | 11.9 | 11.3 | 11.5 | 11.0 | 13.5 | 12.3 | 12.7 |
| lysine | Isoform 2 | | | | | | | | |
| clipped | | | | | | | | | |
| variant (1K) | | | | | | | | | |
| Double | Basic | 2.9 | 3.1 | 2.6 | 2.5 | 2.5 | 3.4 | 2.9 | 2.9 |
| lysine | Isoform 1 | | | (<LOQ) | (<LOQ) | (<LOQ) | | | |
| variant (2K) | | | | | | | | | |

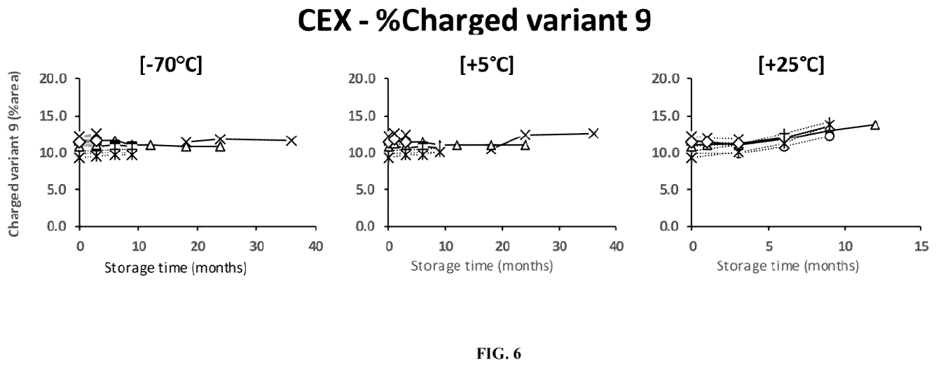

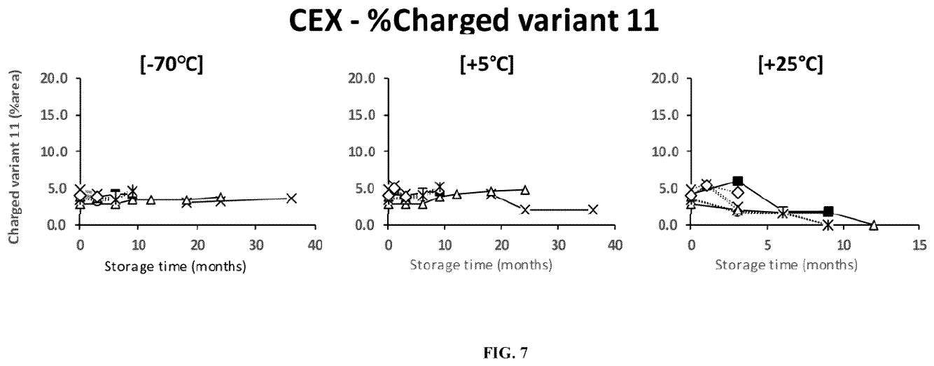

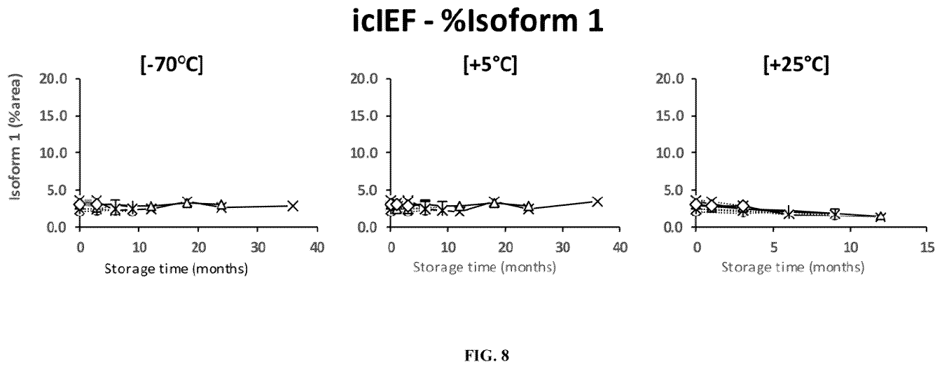

Further, during long-term stability studies, no changes for CEX peaks 9 and 11 or icIEF isoforms 1 or 2 were observed at −70° C., +5° C. and +25° C., as shown in FIG. 6 through FIG. 9. Accordingly, the C-terminal lysine variants were concluded to be quality attributes that contribute to product heterogeneity, with no effect on potency.

Three deamidation sites were identified in members of the population of Fc antagonist molecules during reduced tryptic peptide mapping followed by RP-LC/MS detection (see Table 15). These three identified deamidation sites were GFYPSDIAVEWESNGQPENNYK (SEQ ID NO: 24) (T19; amino acid positions 151-172), VVSVLTVLHQDWLNGK (SEQ ID NO: 25) (T08, amino acid position 082-097), and NQVSLTCLVK (SEQ ID NO: 26) (T18, amino acid positions 141-150), and are all not involved in FcRn binding.

### Example 3: Prevalence of Post-Translational Modifications in Fc Domains

The prevalence of post-translational modifications in Fc domains of the FcRn antagonist reference samples was estimated by comparing the ion intensities of modified peptides to the ion intensities of unmodified peptides.

For tryptic digestion, test samples were denatured in urea prior to reduction with dithiothreitol (DTT). The resulting free thiol groups were alkylated using sodium iodoacetate. The samples were then digested with trypsin and the resulting peptides analyzed using RPLC coupled to UV and MS detection (ESI Q-TOF).

Measured signals were matched onto the sequence using the BioConfirm algorithm incorporated in the MassHunter software. Mass tolerance for matching experimental data onto the sequence was set at 20 ppm. For peptide mapping in reducing conditions, methionine and tryptophan oxidation, asparagine and glutamine deamidation and complex N-glycans (G0, G0F, G1F, G2F, Man5) were considered as variable modifications while cysteine carbamidomethylation (sample preparation related) was considered as a fixed modification. Enzyme specified was trypsin (C-terminal cleavage at lysine or arginine) and 0-2 missed cleavages were allowed. Peak areas from extracted ion chromatograms obtained at 20 ppm mass accuracy were used for quantifying modifications. Aspartate- and isoaspartate-containing peptides have the same m/z but can be discriminated based on chromatographic retention time with the isoaspartate eluting before the aspartate containing peptide.

For chymotryptic digestion, test samples were denatured in RapiGest prior to reduction with dithiothreitol (DTT). The resulting free thiol groups were alkylated using iodoacetamide. The samples were then digested with chymotrypsin and the resulting peptides analyzed using RPLC coupled to UV and MS detection (ESI Q-TOF).

Measured signals were matched onto the sequence using the BioConfirm algorithm incorporated in the MassHunter software. Mass tolerance for matching experimental data onto the sequence was set at 20 ppm. For the reduced chymotryptic peptide mapping, the N-terminal truncations were considered as variable modifications while cysteine carbamidomethylation (sample preparation related) was considered as a fixed modification. Enzyme specified was chymotrypsin (C-terminal cleavage at tyrosine, tryptophan and phenylalanine) and 0-2 missed cleavages were allowed. Peak areas from extracted ion chromatograms obtained at 20 ppm mass accuracy were used for quantifying modifications.

### N-Terminal Modifications

The evaluation of potential processing occurring on the N-terminal (e.g., truncation) using the current sample preparation is hampered by the fact that trypsin cleaves at K and R. Therefore, an additional digest was performed using chymotrypsin, which has different preferential cleavage sites.

Using chymotrypsin, three N-terminal variants were identified at low abundance (<1%): -D, -DK, and -DKTH.

The -DKTH truncated peptide (TCPPCPAPELLGGPSVFLFPPKPK (SEQ ID NO: 27), 005-028) was also identified using tryptic digest. Levels of this peptide are shown in Table 12. Similar low levels (<1.6%) of this peptide were found for all samples tested.

TABLE 12
Relative Quantification of N-Terminal Modifications.
Quantification Based on Extracted Ion MS Chromatograms

| | Ret. | | | | | | |
|---|---|---|---|---|---|---|---|
| | time, | | | Modifi- | | | |
| Sequence | min¹ | Mass, Da¹ | Position | cation | Batch 3 | Batch 1 | Batch 2 |
| DKTHTCPPCPAPELLGGPSVFL | 27.5 | 3086.5712 | 001-028 | Mis- | 98.4% | 98.4% | 98.5% |
| FPPKPK (SEQ ID NO: 28) | | | | cleaved | | | |
| THTCPPCPAPELLGGPSVFLFP | 28.1 | 2843.4491 | 003-028 | | | | |
| PKPK (SEQ ID NO: 29) | | | | | | | |
| TCPPCPAPELLGGPSVFLFPPK | 29.0 | 2605.3378 | 005-028 | DKTH | 1.6% | 1.6% | 1.5% |
| PK (SEQ ID NO: 27) | | | | loss | | | |

### C-Terminal Modifications

Table 13 presents the C-terminal peptide species present in the population of Fc antagonist molecules. The main C-terminal peptide was lysine (K) clipped. Levels of K-truncation range from 88.0% to 89.0%. The lysine truncation was similar for all reference standards. Another C-terminal variant was identified as SLSLSP (caused by C-terminal-GK truncation and P amidation) at a level of approximately 0.5%.

TABLE 13
Relative Quantification of C-Terminal Modifications
Based on Extracted Ion MS Chromatograms

| | Ret. | | | | | | |
|---|---|---|---|---|---|---|---|
| | time, | | | | | | |
| Sequence | min¹ | Mass, Da¹ | Position | Modification | Batch 3 | Batch 1 | Batch 2 |
| SLSLSPGK (SEQ | 17.0 | 787.4442 | 220-227 | — | 10.6% | 10.6% | 10.4% |
| ID NO: 30) | | | | | | | |
| SLSLSP (SEQ | 18.0 | 601.3418 | 220-225 | GK clipped + | 0.5% | 0.5% | 0.5% |
| ID NO: 31) | | | | P amidated | | | |
| SLSLSPG (SEQ | 18.3 | 659.3486 | 220-226 | K clipped | 88.8% | 88.8% | 89.0% |
| ID NO: 32) | | | | | | | |

### Deamidation and Isomerization

Identified aspartate (D) isomerization and asparagine (Q) deamidation sites are presented in Table 14 and Table 15.

Two peptides were found isomerized, peptide 055-068 and 173-189. The percentage of isomerization was relatively low (<1%) and was comparable for the reference standards.

Three peptides were found deamidated, peptide 082-097, 141-150 and 151-172. The levels of deamidation were comparable between reference standards and were highest for peptide 151-172.

TABLE 14
Relative Quantification of Aspartate Isomerization
Based on Extracted Ion MS Chromatograms

| | Ret. | | | | | | |
|---|---|---|---|---|---|---|---|
| | time, | | | Modifi- | | | |
| Sequence | min¹ | Mass, Da¹ | Position | cation | Batch 3 | Batch 1 | Batch 2 |
| TTPPVLDSDGSFFLYSK | 25.6 | 1872.9098 | 173-189 | isoD | 0.6% | 0.7% | 0.6% |
| (SEQ ID NO: 33) | | | | | | | |
| TTPPVLDSDGSFFLYSK | 26.0 | 1872.9144 | 173-189 | — | 99.4% | 99.3% | 99.4% |
| (SEQ ID NO: 33) | | | | | | | |
| FNWYVDGVEVHNAK | 22.1 | 1676.7922 | 055-068 | isoD | 1.2% | 1.3% | 1.3% |
| (SEQ ID NO: 34) | | | | | | | |
| FNWYVDGVEVHNAK | 22.4 | 1676.7951 | 055-068 | — | 98.8% | 98.7% | 98.7% |
| (SEQ ID NO: 34) | | | | | | | |

TABLE 15
Relative Quantification of Deamidation Based on Extracted Ion MS Chromatograms

| | Ret. | | | | | | |
|---|---|---|---|---|---|---|---|
| | time, | | | | | | |
| Sequence | min¹ | Mass, Da¹ | Position | Modification | Batch 3 | Batch 1 | Batch 2 |
| VVSVLTVLHQDWLNGK | 28.8 | 1806.9989 | 082-097 | — | 96.8% | 96.7% | 96.5% |
| (SEQ ID NO: 25) | | | | | | | |
| VVSVLTVLHQDWLNGK | 29.0 | 1807.9820 | 082-097 | Deamidation | 3.2% | 3.3% | 3.5% |
| (SEQ ID NO: 25) | | | | | | | |
| NQVSLTCLVK (SEQ | 21.8 | 1160.6217 | 141-150 | — | 97.8% | 97.5% | 97.5% |
| ID NO: 26) | | | | | | | |
| NQVSLTCLVK (SEQ | 22.0 | 1161.6056 | 141-150 | Deamidation | 2.2% | 2.5% | 2.5% |
| ID NO: 26) | | | | | | | |
| GFYPSDIAVEWESNGQPE | 24.3 | 2543.1231 | 151-172 | — | 92.6% | 92.4% | 92.1% |
| NNYK (SEQ ID NO: 24) | | | | | | | |
| GFYPSDIAVEWESNGQPE | 24.4 | 2544.1066 | 151-172 | Deamidation | 7.4% | 7.6% | 7.9% |
| NNYK (SEQ ID NO: 24) | | | | | | | |

### Oxidation

Identified oxidation sites are presented in Table 16. Methionine oxidation in peptide 197-213 was found at levels ranging from 0.7% to 1.0% for the reference samples analyzed. Tryptophan oxidation was not observed.

TABLE 16
Relative Quantification of Methionine Oxidation
Based on Extracted Ion MS Chromatograms

| | Ret. | | | | | | |
|---|---|---|---|---|---|---|---|
| | time, | | | | | | |
| Sequence | min¹ | Mass, Da¹ | Position | Modification | Batch 3 | Batch 1 | Batch 2 |
| WQQGNVFSCSVMHEA | 18.0 | 2035.9127 | 197-213 | Methionine | 0.7% | 1.0% | 1.0% |
| LK (SEQ ID NO: 35) | | | | oxidation | | | |
| WQQGNVFSCSVMHEA | 20.3 | 2019.9185 | 197-213 | — | 99.3% | 99.0% | 99.0% |
| LK (SEQ ID NO: 35) | | | | | | | |

### N-Glycosylation

Table 17 presents N-glycosylation site occupancy (Asn77 in peptide 073-081). The overall site occupancy was 97.8% to 97.9% across the three reference standards. The predominant glycoform was GOF (59.2% to 64.3%). The different glycoforms observed align well with the glycoforms observed in the RPLC-MS analyses and N-linked oligosaccharide profiling.

TABLE 17
Relative Quantification of N-Glycosylation Based on
Extracted Ion MS Chromatograms

| | Ret. | | | | | | |
|---|---|---|---|---|---|---|---|
| | time, | | | Modifi- | | | |
| Sequence | min¹ | Mass, Da¹ | Position | cation | Batch 3 | Batch 1 | Batch 2 |
| EEQYNSTYR (SEQ | 10.1 | 1188.4968 | 073-081 | — | 2.3% | 2.2% | 2.1% |
| ID NO: 36) | | | | | | | |
| EEQYNSTYR (SEQ | 9.7 | 2486.9624 | 073-081 | G0 | 4.7% | 4.0% | 4.0% |
| ID NO: 36) | | | | | | | |
| EEQYNSTYR (SEQ | 9.7 | 2633.0198 | 073-081 | G0F | 64.3% | 59.5% | 59.2% |
| ID NO: 36) | | | | | | | |
| EEQYNSTYR (SEQ | 9.7 | 2783.0726 | 073-081 | G1F | 22.4% | 25.6% | 25.9% |
| ID NO: 36) | | | | | | | |
| EEQYNSTYR (SEQ | 9.6 | 2957.1212 | 073-081 | G2F | 6.3% | 8.7% | 8.9% |
| ID NO: 36) | | | | | | | |

| | | | |
|---|---|---|---|
| N-glycosylation site occupancy | 97.7% | 97.8% | 97.9% |

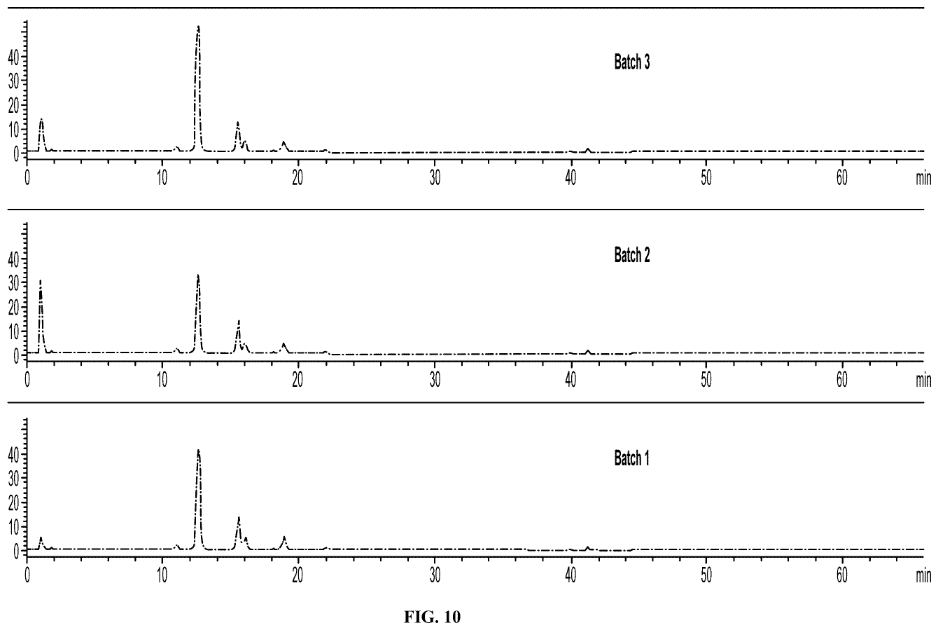

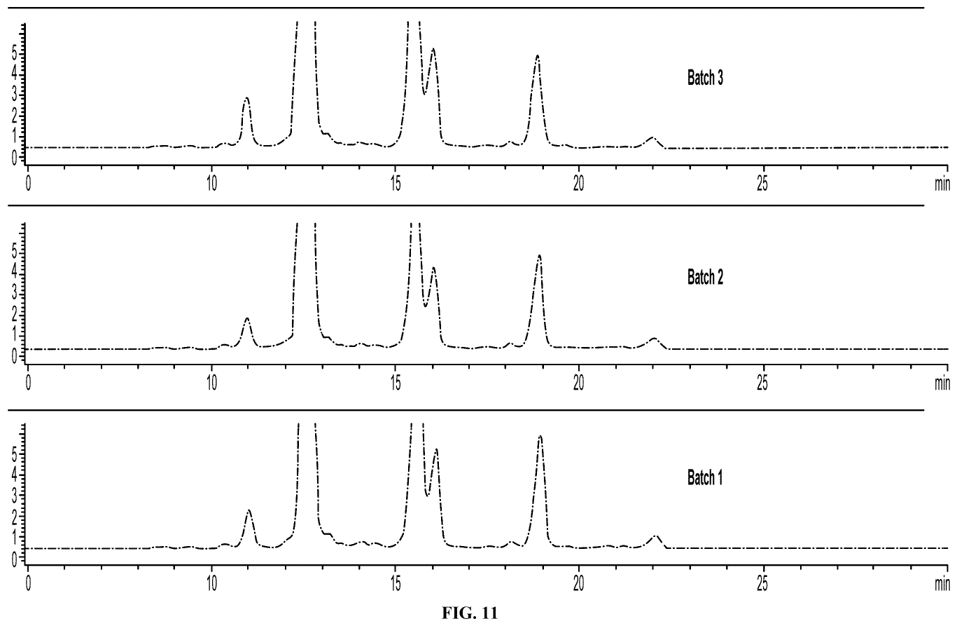

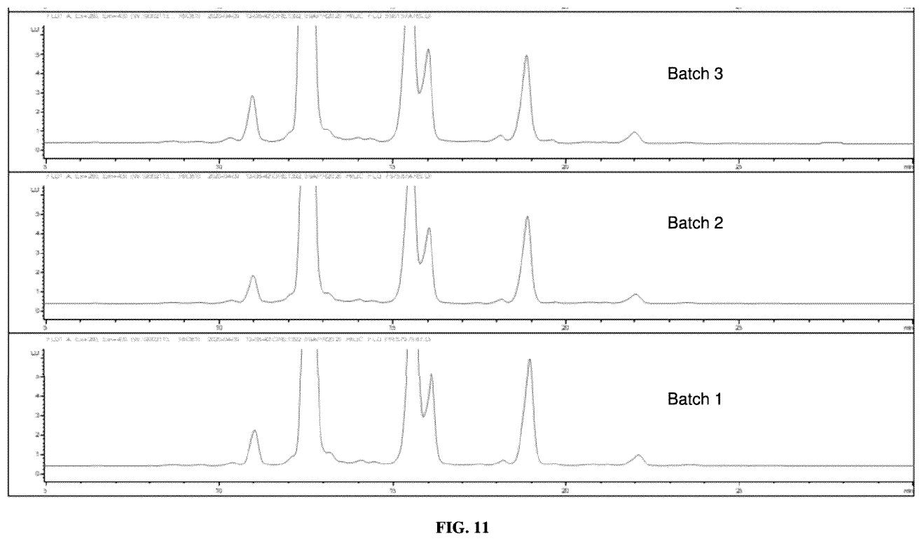

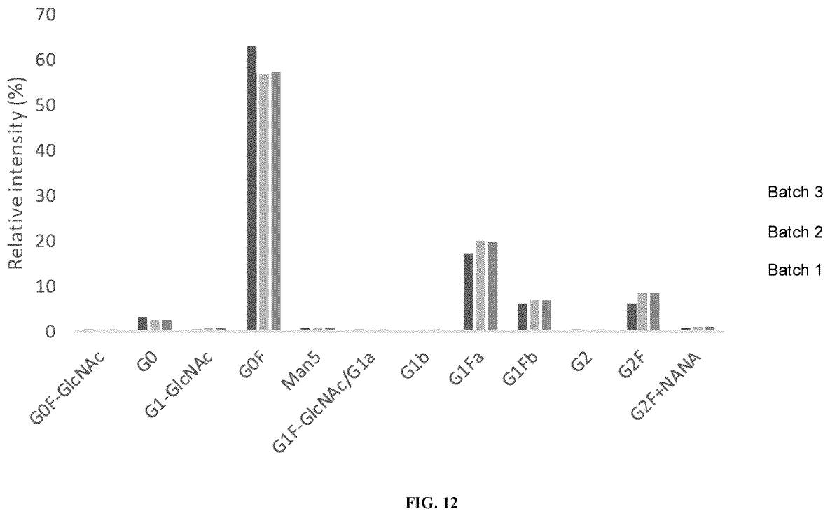

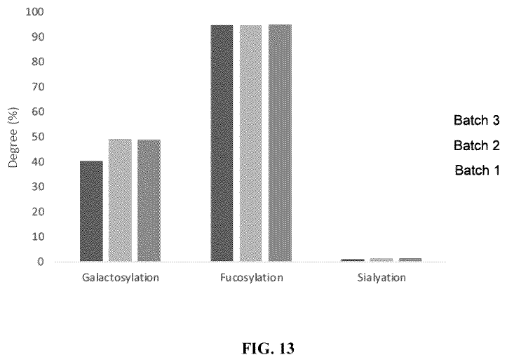

Hydrophilic interaction liquid chromatography (HILIC) fluorescence chromatograms of the 2-aminobenzamide (2-AB) labeled N-glycans originating from the reference samples are shown in FIG. 10 and FIG. 11. Peak identification was the result of mass spectrometric data interpretation and chromatographic elution together with glycan biosynthetic rules. Quantification was based on fluorescence peak areas. The N-glycan identity and relative intensity is shown in Table 18, with FIG. 12 showing the same data in a graphical representation. The degree of galactosylation and fucosylation is shown in Table 19. A graphical representation is shown in FIG. 13.

TABLE 18
Identity And Relative Intensity of the 2-AB-labeled Glycans
(▪ N-acetylglucosamine, ○ Mannose, ● Galactose, ∇ Fucose) for All Peaks of the Reference Samples

| | | | Batch | Batch | Batch |
|---|---|---|---|---|---|
| Glycan | Symbol | Structure | 3 | 2 | 1 |
| Man3GlcNAc3Fuc | G0F-GlcNAc | 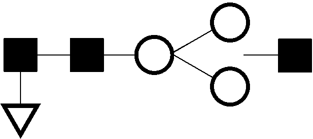 | 0.4 | 0.4 | 0.4 |
| Man3GlcNAc4 | G0 | 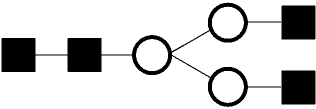 | 3.2 | 2.7 | 2.7 |
| GalMan3GlcNAc3 | G1-GlcNAc | 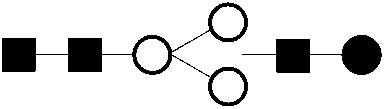 | 0.6 | 0.8 | 0.7 |
| Man3GlcNAc4Fuc | G0F | 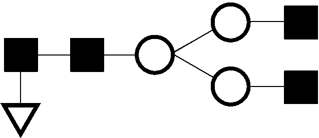 | 63.1 | 57.1 | 57.4 |
| Man5GlcNAc2 | Man5 | 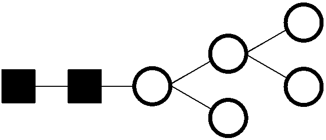 | 0.7 | 0.8 | 0.8 |
| GalMan3GlcNAc3Fuc/ GalMan3GlcNAc4 | G1F-GlcNAc/ G1a | 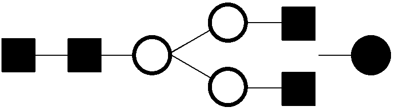 | 0.5 | 0.6 | 0.6 |
| GalMan3GlcNAc4 | G1b | 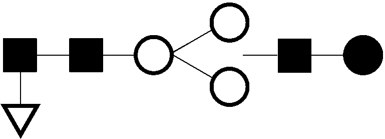 | 0.3 | 0.4 | 0.4 |
| GalMan3GlcNAc4Fuc | G1Fa | 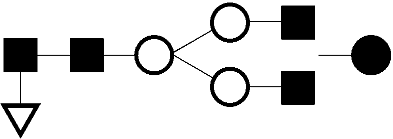 | 17.3 | 20.0 | 19.9 |
| GalMan3GlcNAc4Fuc | G1Fb | | 6.2 | 6.9 | 6.9 |
| Gal2Man3GlcNAc4 | G2 | 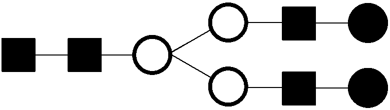 | 0.5 | 0.6 | 0.6 |
| Gal2Man3GlcNAc4Fuc | G2F | 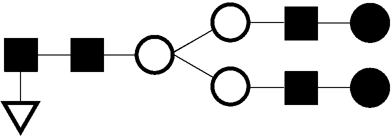 | 6.2 | 8.4 | 8.4 |
| NANAGal2Man3GlcNAc4Fuc | G2F + NANA | 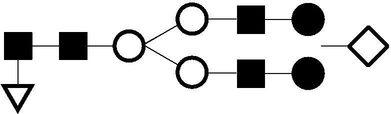 | 0.9 | 1.2 | 1.2 |

TABLE 19
Degree of Galactosylation and Fucosylation
of the Reference Samples At Glycan Level

| Level (%) | Batch 3 | Batch 2 | Batch 1 |
|---|---|---|---|
| Galactosylation | 40.2 | 49.1 | 48.7 |
| Fucosylation | 94.7 | 94.7 | 94.9 |
| Sialyation | 0.9 | 1.2 | 1.2 |

All samples had similar profiles, with GOF as predominant glycoform, followed by GIF and G2F. Lower levels of non-fucosylated N-glycans, Man5, addition of sialic acid and loss of GlcNac were detected at comparable levels in all samples. The reference sample Batch 3 had a slightly lower degree of galactosylation, when compared to the other samples. The degree of fucosylation was between 94.7% and 94.9% for all samples tested.

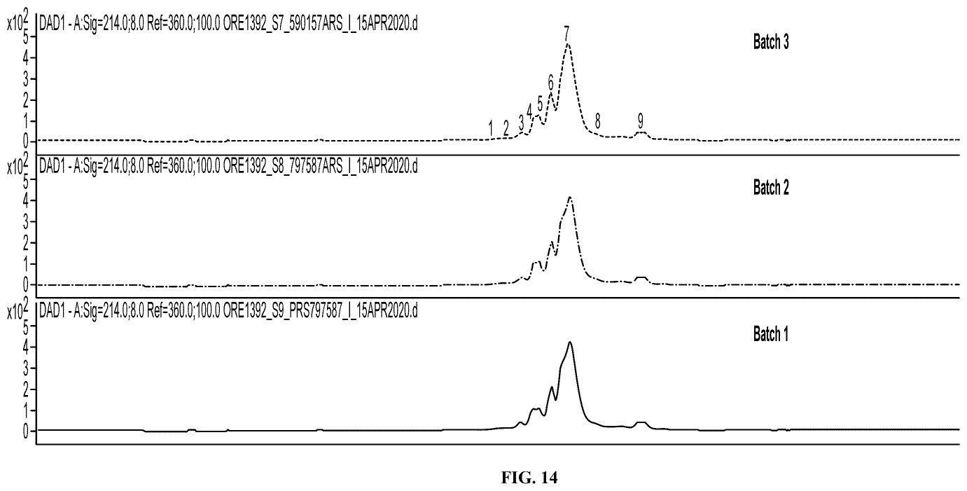

RPLC-UV 214 nm spectra of the intact reference standards are shown in FIG. 14. The identity and mass accuracy of the annotated peaks, as obtained from the deconvoluted MS spectra for each peak, is presented in Table 20.

TABLE 20
Results for Intact ESI-MS of Primary Reference Standard Batch 1

| Molecular | | | |
|---|---|---|---|
| Weight | | | |
| Species | Detected | Theoretical | |
| (Peak No) | Mass (Da) | Mass (Da) | Identity |
| a | (Traces) | 26957.44 | Reduced Fc (missing |
| | | | interchain disulfide |
| | | | bridges resulting in |
| | | | intrachain disulfide |
| | | | bridge) |
| 1 | (Traces) | 53914.89 | On-column oxidation reduced |
| | | | variant ¹ |
| 2 | 54190.6 | 54189.24 | Fc/2K + 18 Da ¹ |
| | 53950.7 | 53950.89 | Fc/0K + 36 Da ¹ |
| 3 | 54061.2 | 54061.06 | Fc/1K + 18 Da ¹ |
| 4 | 54171.4 | 54171.24 | Fc/2K |
| 5 | 53933.4 | 53932.89 | Fc/0K + 18 Da ¹ |
| 6 | 54043.2 | 54043.06 | Fc/1K |
| 7 | 53915.1 | 53914.89 | Fc/0K |
| (main | | | |
| isoform) | | | |
| 8 | 53671.7 | 53671.62 | Loss of DK at N-terminus of |
| | | | Fc/0K |
| | 53869.8 | — | Unknown variant (loss of |
| | | | approx. 45 Da) |
| 9 | 52469.5 | 52469.53 | Partially unglycosylated Fc/0K |
| | 53917.2 | 53916.90 | Fc/0K with one disulfide bond |
| | | | missing |

Da = Dalton;
Fc/0K = double lysine clipped;
Fc/1K = single lysine clipped;
Fc/2K = non-lysine clipped
¹ Possible method induced variant.

The predominant mass detected was comparable to the calculated theoretical predominant intact mass for the population of Fc antagonist molecules of 53,915 Da based on the double C-terminal lysine clipped amino acid sequence with two GOF glycans each at 100% occupancy. The other peaks were identified as C-terminal lysine clipped variants (single or double clipped), N-terminal loss of DK, addition of +18 Da or +36 Da (possible method induced addition of water), a reduced variant (missing interchain disulfide bridges), on-column re-oxidation of the reduced variant, partially unglycosylated Fc and Fc with a disulfide bond missing.

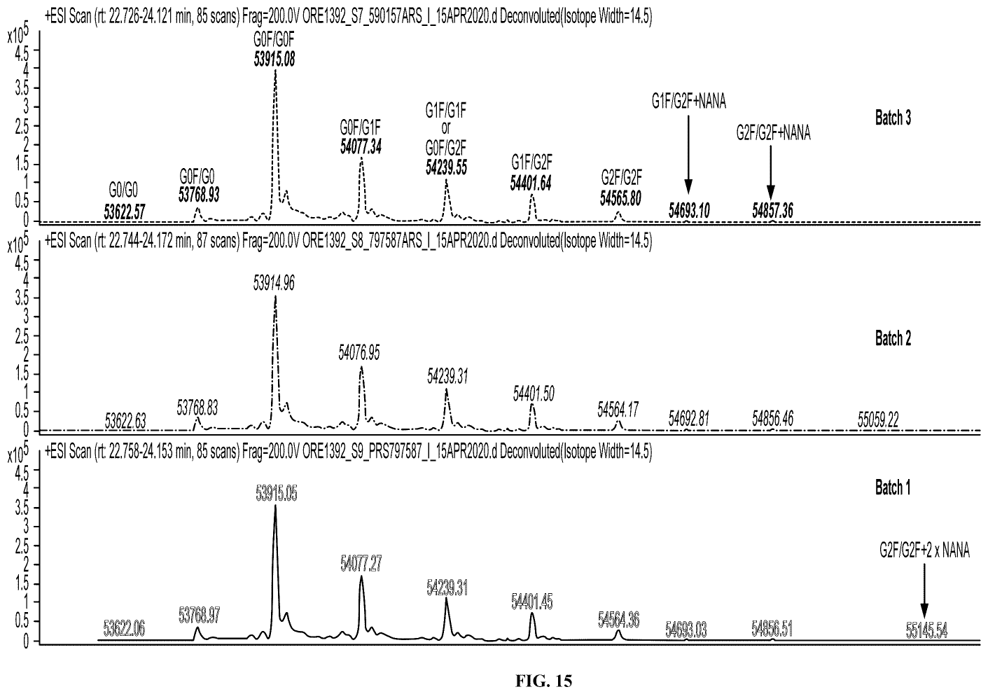

The deconvoluted spectra (with N-glycoforms annotated) of the main peak in the RPLC profiles (i.e., the double C-terminal lysine clipped variant) are shown in FIG. 15. The measured molecular weight and mass accuracy for each glycoform is shown in Table 21.

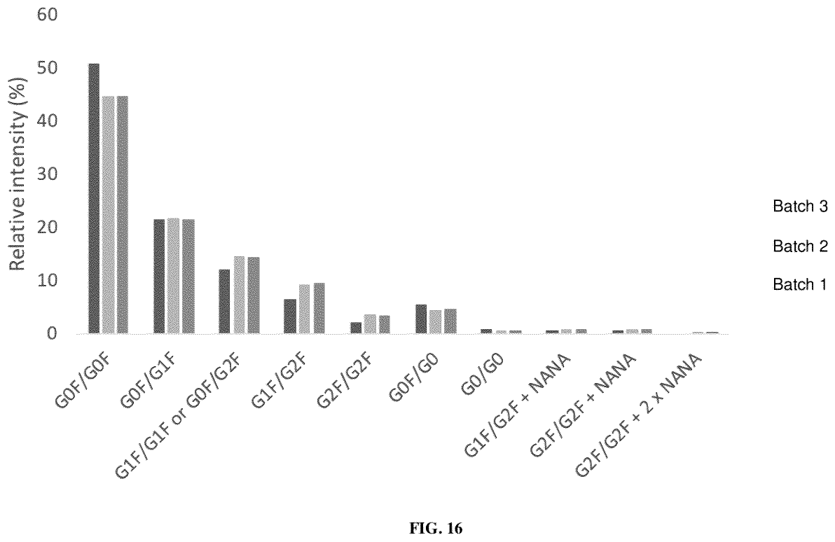

The relative intensity for each glycoform is shown in Table 22. Graphical representation of the relative amounts of the various N-glycans for all samples is provided in FIG. 16.

TABLE 21
Measured Molecular Weight and the Corresponding Mass Accuracy
for the Glycoforms of the Intact Reference Samples

| Fc Antagonist Molecules | | Batch 3 | Batch 2 | Batch 1 |
|---|---|---|---|---|
| G0F/G0F | Measured Mass (Da) | 53915.08 | 53914.96 | 53915.05 |
| Theoretical mass | Mass Accuracy (ppm) | −3.6 | −1.3 | −3.0 |
| 53914.89 Da | | | | |
| G0F/G1F | Measured Mass (Da) | 54077.34 | 54076.95 | 54077.27 |
| Theoretical mass | Mass Accuracy (ppm) | −5.7 | 1.5 | −4.4 |
| 54077.03 Da | | | | |
| G0F/G2F or G1F/G1F | Measured Mass (Da) | 54239.55 | 54239.31 | 54239.31 |
| Theoretical mass | Mass Accuracy (ppm) | −6.9 | −2.5 | −2.5 |
| 54239.17 Da | | | | |
| G1F/G2F | Measured Mass (Da) | 54401.64 | 54401.5 | 54401.45 |
| Theoretical mass | Mass Accuracy (ppm) | −5.9 | −3.4 | −2.4 |
| 54401.32 Da | | | | |
| G2F/G2F | Measured Mass (Da) | 54564.8 | 54564.17 | 54564.36 |
| Theoretical mass | Mass Accuracy (ppm) | −24.6 | −13.0 | −16.5 |
| 54563.46 Da | | | | |
| G0F/G0 | Measured Mass (Da) | 53768.93 | 53768.83 | 53768.97 |
| Theoretical mass | Mass Accuracy (ppm) | −3.5 | −1.6 | −4.2 |
| 53768.74 Da | | | | |
| G0/G0 | Measured Mass (Da) | 53622.57 | 53622.63 | 53622.06 |
| Theoretical mass | Mass Accuracy (ppm) | 0.6 | −0.6 | 10.1 |
| 53622.60 Da | | | | |
| G1F/G2F + NANA | Measured Mass (Da) | 54693.1 | 54692.81 | 54693.03 |
| Theoretical mass | Mass Accuracy (ppm) | −9.6 | −4.3 | −8.3 |
| 54692.58 Da | | | | |
| G2F/G2F + NANA | Measured Mass (Da) | 54857.36 | 54856.46 | 54856.51 |
| Theoretical mass | Mass Accuracy (ppm) | −48.1 | −31.7 | −32.6 |
| 54854.72 Da | | | | |
| G2F/G2F + 2 × NANA | Measured Mass (Da) | Traces | 55146.05 | 55145.54 |
| Theoretical mass | Mass Accuracy (ppm) | / | −1.3 | 8.0 |
| 55145.98 Da | | | | |
| Man5/Man5 | Measured Mass (Da) | Traces | Traces | Traces |
| Theoretical mass | Mass Accuracy (ppm) | / | / | / |
| 53458.39 Da | | | | |

TABLE 22
Relative Intensity of the Glycoforms
of the Intact Reference Samples

| Relative Intensity (%) | Batch 3 | Batch 2 | Batch 1 |
|---|---|---|---|
| G0F/G0F | 50.8 | 44.6 | 44.6 |
| G0F/G1F | 21.4 | 21.6 | 21.4 |
| G1F/G1F or G0F/G2F | 11.9 | 14.5 | 14.3 |
| G1F/G2F | 6.4 | 9.2 | 9.6 |
| G2F/G2F | 2.2 | 3.6 | 3.4 |
| G0F/G0 | 5.4 | 4.5 | 4.7 |
| G0/G0 | 0.7 | 0.5 | 0.5 |
| G1F/G2F + NANA | 0.5 | 0.8 | 0.8 |
| G2F/G2F + NANA | 0.6 | 0.7 | 0.7 |
| G2F/G2F + 2 × NANA | Traces | 0.1 | 0.1 |
| Man5/Man5 | Traces | Traces | Traces |

It can be concluded from the graph that the predominant glycoform was GOF/GOF, with minor differences in N-glycosylation observed between the samples.

### Example 4: Properties of Pharmaceutical Formulations

### Aggregation

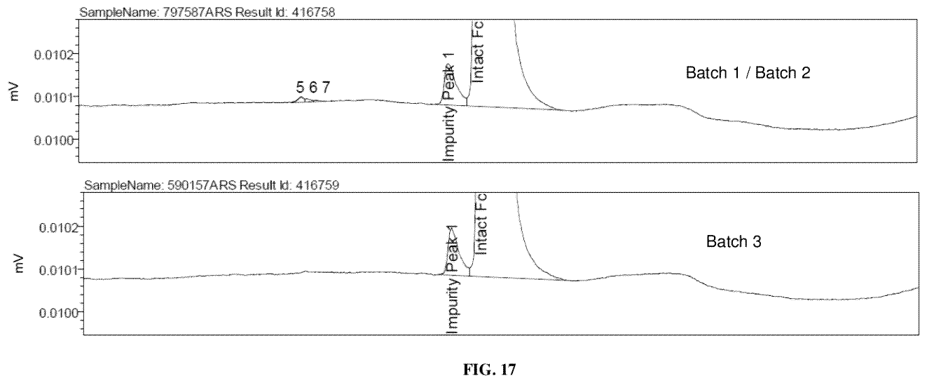

GP HPLC was performed to determine the levels of monomer and aggregate species present in the FcRn antagonist reference samples, and to evaluate the conformation of the protein under non-denaturing conditions. The results are shown in Table 23 and FIG. 17. In all three reference standards the percentage of monomers was 99.6% and the percentage of aggregate was 0.4%.

TABLE 23
Results for GP HPLC Testing of Reference Standards

Relative %

| Species | Batch 3 | Batch 2/Batch 1 |
|---|---|---|
| Aggregate 1 | 0.4 | 0.4 |
| Monomer | 99.6 | 99.6 |

LOQ = 0.1%,
LOD = 0.04%

### Disulfide Bonding

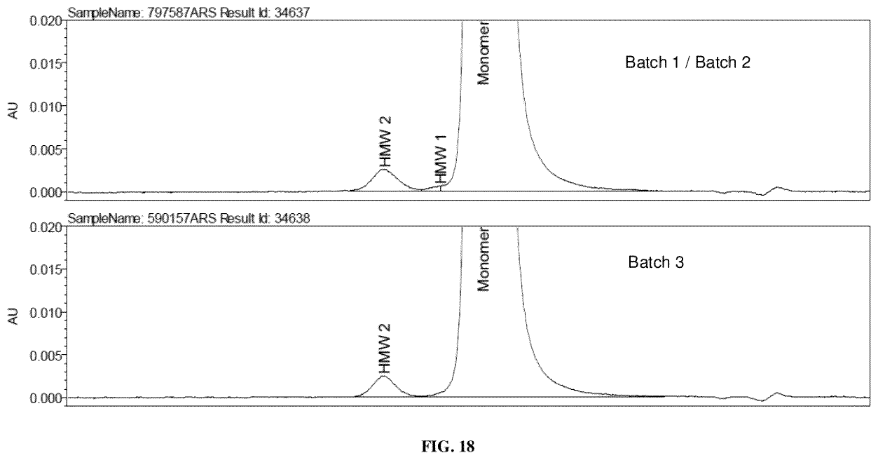

Under non-reducing conditions, as shown in FIG. 18 and Table 24, a major peak corresponding to the molecular weight of an intact Fc region was visible in the electropherogram. The purity under non-reducing conditions (% intact Fc) was 98.2%. Several minor peaks migrating before the IgG peak were visible in an expanded view of the electropherogram for primary reference standard Batch 1 and working reference standard Batch 2, corresponding most likely with reduced (single Fc domain) fragments.

TABLE 24
Results for Non-Reducing CE SDS Testing of Reference Standards

Percentage Area (%)

| Peak | Batch 3 | Batch 2/Batch 1 |
|---|---|---|
| 1 | Not detected | Not detected |
| 2 | Not detected | Not detected |
| 3 | Not detected | Not detected |
| 4 | Not detected | Not detected |
| 5 | Not detected | 0.1 (<LOQ) |
| 6 | Not detected | 0.1 (<LOQ) |
| 7 | Not detected | <0.1 (<LOD) |
| 8 | Not detected | Not detected |
| 9 | Not detected | Not detected |
| 10 (Impurity | 1.8 | 1.6 |
| peak 1) | | |
| 11 (intact Fc) | 98.2 | 98.2 |

### Mass and Mass Heterogeneity

SEC-MALS was performed to further confirm the molecular mass distribution and relative quantities of the monomeric and aggregate species observed by the GP HPLC method coupled with UV detection. The ability of the separated components to scatter light, measured using a light scatter detector, was used to estimate molecular masses.

The monomeric molecular mass determined by size exclusion chromatography multi angle light scattering (SEC-MALS) for the reference standards was comparable to the theoretical molecular mass of 53,915 Da, and consistent with the results obtained by GP HPLC (See Table 25, and FIG. 18).

TABLE 25
Results for Monomeric Mass Heterogeneity
Analysis of Reference Standards

Result

| | | |
|---|---|---|
| Condition | Batch 3 | Batch 2/Batch 1 |

High Molecular Weight 2

| | | |
|---|---|---|
| Weight average molecular | 161.0 | 141.1 |
| weight (kDa) | | |
| Apex molecular weight (kDa) | 134.7 | 121.7 |
| Relative % content | 0.4 | 0.4 |

High Molecular Weight 1

| | | |
|---|---|---|
| Weight average molecular | ND | 110.4 |
| weight (kDa) | | |
| Apex molecular weight (kDa) | ND | 93.1 |
| Relative % content | ND | <0.1 |

Monomer

| | | |
|---|---|---|
| Weight average molecular | 54.6 | 54.5 |
| weight (kDa) | | |
| Apex molecular weight (kDa) | 55.2 | 55.2 |
| Relative % content | 99.6 | 99.5 |

ND: Not detected

### Free Thiol

The free thiol content of the reference standards was determined using Ellman's reagent. To determine the presence of any free cysteine residues within the internal structure of the protein, the assay was also performed after denaturation of the antibody.

Under native conditions, the free thiol levels were below the calculated limit of quantitation (LOQ) of the method (Table 26). Under denaturation conditions, slightly higher free thiol levels were detected for primary reference standard Batch 1/working reference standard Batch 2 compared to the reference standard Batch 3. However, these levels of free thiol were considered to be low and close to the LOQ of the method.

TABLE 26
Free Thiol Assay Results for Reference Samples

| | | | | |
|---|---|---|---|---|
| Sample | Attribute | Batch 3 | Batch 2 | Batch 1 |

| | | | |
|---|---|---|---|
| Native sample | Free thiol content | <LOQ | <LOQ |
| | (mmol/mol protein) | | |
| | % free cysteine | <LOQ | <LOQ |
| Denatured | Free thiol content | <LOQ | 113.7 |
| | (mmol/mol protein) | | |
| | % free cysteine | <LOQ | 0.95 |

### Example 5: Batch Variability of Compositions Comprising FcRn Antagonist Molecule Variants

Compositions comprising a population of Fc antagonist molecules were characterized, and their constituents determined similar to Example 1.

The charge heterogeneity of the samples was assessed using strong cation exchange high performance liquid chromatography (CEX HPLC). Proteins present in the samples were separated and quantified according to their surface charge distribution, based on the interaction of charges on the surface of the protein with charged groups on the surface of the column. Proteins are positively charged in buffers with a pH value below their pI. Proteins were eluted from the column using a sodium chloride gradient (mobile phase B), with acidic species eluting first, followed by more basic species. Separated components passed through a UV detector cell and the absorbance was measured at a wavelength of 220 nm. The results are shown in Tables 26 & 27.

Peaks of eluted species were classified as acidic or basic based on their elution time relative to the main peak in the CEX profile. Peaks eluting earlier than the main peak (peak 8) were identified as acidic species (peaks 1 to 7) and peaks eluting later (peaks 9 to 12) were identified as basic species. A total of twelve (12) charged species were identified by CEX for all samples tested. Consistent with the results in Example 1, the main isoform is present at a relative percentage area of approximately 64%. Total basic and acidic isoforms account for about 16% and 19% relative percentage area, respectively. The identity of the different CEX isoform peaks is presented in FIG. 2 and Table 6.

The charge heterogeneity of the samples was also evaluated by imaged capillary isoelectric focusing (icIEF) and results are shown in Tables 27 & 28.

As in Example 1, the peaks were numbered based on their measured isoelectric point (pI) relative to the major profile peak. Peaks displaying a pI at a pH lower than that of the major peak were identified as acidic species, and peaks displaying a pI at a higher pH were identified as basic species.

Six isoforms were detected across a pI range of pI 6.7 to 7.6. The main isoform was detected with a pI of approximately 7.2 and a relative percentage area of about 65%. Total basic isoforms accounted for about 16% relative percentage area and total acidic isoforms accounted for about 18% relative percentage area. The identity of the different isoforms is presented in Table 8.

TABLE 27
icIEF & CEX Results for FcRn Antagonist Drug Substance

| | | | | | Std | Lower | Upper | Lower | Upper |
|---|---|---|---|---|---|---|---|---|---|
| Parameter | N | Min | Max | Mean | Dev | 95% TI | 95% TI | 99% TI | 99% TI |
| icIEF - % Main isoform | 144 | 60.6 | 71.0 | 65.2 | 1.921 | 59.7 | 70.7 | 59.4 | 70.9 |
| icIEF - % Acidic isoforms | 144 | 16.4 | 24.5 | 18.8 | 1.321 | 15.0 | 22.6 | 14.8 | 22.8 |
| icIEF - % isoform 1 | 134 | 1.7 | 4.6 | 3.2 | 0.574 | 1.5 | 4.8 | 1.5 | 4.9 |
| icIEF - % isoform 2 | 134 | 8.6 | 16.2 | 12.9 | 1.182 | 9.5 | 16.3 | 9.4 | 16.5 |
| icIEF - % Basic isoforms | 144 | 10.3 | 20.8 | 16.1 | 1.812 | 10.9 | 21.3 | 10.6 | 21.5 |
| icIEF - % isoform 4 | 134 | 11.1 | 15.8 | 12.7 | 0.864 | 10.2 | 15.2 | 10.1 | 15.3 |
| icIEF - % isoform 5 | 134 | 2.9 | 6.4 | 4.4 | 0.585 | 2.8 | 6.1 | 2.7 | 6.2 |
| icIEF - % isoform 6 | 131 | 0.6 | 4.0 | 1.7 | 0.857 | 0.0 | 4.2 | 0.0 | 4.3 |
| CEX - % Main species | 137 | 56.9 | 68.3 | 64.5 | 2.022 | 58.7 | 70.3 | 58.4 | 70.6 |
| CEX - % Acidic species | 137 | 13.8 | 19.6 | 16.4 | 1.079 | 13.3 | 19.5 | 13.2 | 19.7 |
| CEX - % specie 1 | 134 | 0.7 | 1.7 | 1.3 | 0.217 | 0.6 | 1.9 | 0.6 | 1.9 |
| CEX - % specie 2 | 134 | 0.9 | 1.7 | 1.3 | 0.147 | 0.9 | 1.8 | 0.9 | 1.8 |
| CEX - % specie 3 | 134 | 0.6 | 1.3 | 0.8 | 0.130 | 0.4 | 1.2 | 0.4 | 1.2 |
| CEX - % specie 4 | 134 | 0.8 | 2.0 | 1.3 | 0.229 | 0.6 | 1.9 | 0.6 | 2.0 |
| CEX - % specie 5 | 134 | 1.1 | 2.1 | 1.5 | 0.185 | 0.9 | 2.0 | 0.9 | 2.0 |
| CEX - % specie 6 | 134 | 2.1 | 3.2 | 2.6 | 0.227 | 1.9 | 3.2 | 1.9 | 3.2 |
| CEX - % specie 7 | 134 | 6.8 | 9.4 | 7.7 | 0.436 | 6.4 | 8.9 | 6.4 | 9.0 |
| CEX - % Basic species | 137 | 14.8 | 27.6 | 19.1 | 1.866 | 13.7 | 24.5 | 13.5 | 24.7 |
| CEX - % specie 9 | 134 | 7.0 | 14.0 | 11.5 | 1.132 | 8.2 | 14.7 | 8.1 | 14.9 |
| CEX - % specie 10 | 134 | 1.5 | 5.5 | 2.4 | 0.562 | 0.8 | 4.0 | 0.7 | 4.1 |
| CEX - % specie 11 | 134 | 2.9 | 7.4 | 4.1 | 0.716 | 2.0 | 6.1 | 1.9 | 6.2 |
| CEX - % specie 12 | 134 | 0.4 | 3.2 | 1.2 | 0.524 | 0.0 | 2.7 | 0.0 | 2.7 |

TABLE 28
icIEF & CEX Results for FcRn Antagonist Drug Product

| | | | | | Std | Lower | Upper | Lower | Upper |
|---|---|---|---|---|---|---|---|---|---|
| Parameter | N | Min | Max | Mean | Dev | 95% TI | 95% TI | 99% TI | 99% TI |
| icIEF - % Main isoform | 97 | 61.3 | 68.8 | 65.0 | 1.700 | 60.0 | 70.0 | 59.8 | 70.3 |
| icIEF - % Acidic isoforms | 97 | 15.2 | 23.1 | 18.4 | 1.724 | 13.3 | 23.5 | 13.0 | 23.8 |
| icIEF - % isoform 1 | 95 | 2.4 | 4.3 | 3.3 | 0.428 | 2.0 | 4.5 | 1.9 | 4.6 |
| icIEF - % isoform 2 | 95 | 11.2 | 15.8 | 13.3 | 1.061 | 10.1 | 16.4 | 10.0 | 16.6 |
| icIEF - % Basic isoforms | 97 | 13.9 | 20.1 | 16.6 | 1.445 | 12.3 | 20.8 | 12.1 | 21.1 |
| icIEF - % isoform 4 | 95 | 10.8 | 15.6 | 12.8 | 0.985 | 9.9 | 15.7 | 9.8 | 15.9 |
| icIEF - % isoform 5 | 95 | 2.6 | 5.7 | 4.1 | 0.700 | 2.0 | 6.1 | 1.9 | 6.3 |
| icIEF - % isoform 6 | 95 | 0.7 | 3.6 | 1.5 | 0.577 | 0.0 | 3.2 | 0.0 | 3.3 |
| CEX - % Main species | 94 | 59.5 | 67.9 | 64.4 | 1.874 | 58.8 | 69.9 | 58.5 | 70.2 |
| CEX - % Acidic species | 94 | 13.5 | 19.2 | 16.3 | 1.000 | 13.4 | 19.3 | 13.2 | 19.5 |
| CEX - % specie 1 | 90 | 0.4 | 1.9 | 1.2 | 0.320 | 0.2 | 2.1 | 0.2 | 2.2 |
| CEX - % specie 2 | 90 | 0.9 | 2.0 | 1.3 | 0.173 | 0.8 | 1.8 | 0.8 | 1.9 |
| CEX - % specie 3 | 90 | 0.0 | 1.8 | 0.9 | 0.357 | 0.0 | 2.0 | 0.0 | 2.1 |
| CEX - % specie 4 | 90 | 0.8 | 2.1 | 1.3 | 0.197 | 0.8 | 1.9 | 0.7 | 2.0 |
| CEX - % specie 5 | 90 | 1.0 | 1.9 | 1.5 | 0.144 | 1.0 | 1.9 | 1.0 | 1.9 |
| CEX - % specie 6 | 90 | 2.0 | 3.1 | 2.5 | 0.162 | 2.0 | 3.0 | 2.0 | 3.0 |
| CEX - % specie 7 | 90 | 6.9 | 8.7 | 7.6 | 0.282 | 6.8 | 8.5 | 6.7 | 8.5 |
| CEX - % Basic species | 94 | 16.3 | 24.1 | 19.3 | 1.585 | 14.6 | 24.0 | 14.4 | 24.2 |
| CEX - % specie 9 | 90 | 10.0 | 14.4 | 11.7 | 0.881 | 9.1 | 14.3 | 8.9 | 14.4 |
| CEX - % specie 10 | 90 | 1.4 | 4.9 | 2.4 | 0.615 | 0.6 | 4.3 | 0.5 | 4.4 |
| CEX - % specie 11 | 90 | 3.0 | 6.3 | 4.1 | 0.624 | 2.2 | 5.9 | 2.1 | 6.0 |
| CEX - % specie 12 | 90 | 0.5 | 2.6 | 1.2 | 0.529 | 0.0 | 2.7 | 0.0 | 2.8 |

Batch variation in N-glycan forms within the FcRn antagonist compositions was also analyzed. As described in Example 3, hydrophilic interaction liquid chromatography (HILIC) fluorescence chromatograms of the 2-aminobenzamide (2-AB) labeled N-glycans originating from the samples were analyzed. Peak identification was the result of mass spectrometric data interpretation and chromatographic elution together with glycan biosynthetic rules. Quantification was based on fluorescence peak areas. The N-glycan identity and relative intensity of the samples is shown in Tables 29-35.

TABLE 29
Drug Substance & Reference Standard - Glycan Forms

| | | | | | | Lower | Upper | Lower | Upper |
|---|---|---|---|---|---|---|---|---|---|
| Parameter | N | Min | Max | Mean | Std Dev | 95% TI | 95% TI | 99% TI | 99% TI |
| Glycan form - % G0-GlcNAc | Not detected (ND) - Traces* | | | | | | | | |
| Glycan form - % G0E-GlcNAc | 24 | Traces* | 0.6 | Not applicable | | | | | |
| Glycan form - % G0 | 44 | 2.5 | 4.5 | 3.3 | 0.563 | 1.6 | 5.1 | 1.4 | 5.3 |
| Glycan form - % G1-GlcNAc | 17 | ND | 1.6 | Not applicable | | | | | |
| Glycan form - % GOF | 44 | 53.8 | 64.1 | 57.8 | 2.220 | 50.1 | 1 | 64.9 | 50.1 |
| Glycan form - % Man5 | 44 | 0.7 | 1.4 | 1.0 | 0.149 | 0.5 | 1.5 | 0.5 | 1.5 |
| Glycan form - % G1F-GlcNAc/G1a | 34 | ND | 0.6 | Not applicable | | | | | |
| Glycan form - % G1a | 10 | ND | 0.5 | Not applicable | | | | | |
| Glycan form - % G1b | 38 | Traces* | 0.5 | Not applicable | | | | | |
| Glycan form - % G0FB | 34 | ND | 0.9 | Not applicable | | | | | |
| Glycan form - % G1Fa | 44 | 16.5 | 20.7 | 18.6 | 1.104 | 15.1 | 22.1 | 14.8 | 22.4 |
| Glycan form - % G1Fb | 44 | 6.1 | 7.6 | 6.8 | 0.350 | 5.7 | 7.9 | 5.6 | 8.0 |
| Glycan form - % Man6 | Not detected (ND) - Traces* | | | | | | | | |
| Glycan form - % G2 | 17 | ND | 0.6 | Not applicable | | | | | |
| Glycan form - % G2/G1F + NANA | 8 | ND | 0.5 | Not applicable | | | | | |
| Glycan form - % G1FB | 27 | ND | 1.0 | Not applicable | | | | | |
| Glycan form - % G2F | 44 | 5.6 | 9.2 | 7.7 | 0.793 | 5.2 | 10.2 | 5.0 | 10.4 |
| Glycan form - % G2FB | Not detected (ND) - Traces* | | | | | | | | |
| Glycan form - % G2F + Gal | Not detected (ND) - Traces* | | | | | | | | |
| Glycan form - % G2FS1 | 44 | 0.8 | 1.6 | 1.3 | 0.185 | 0.7 | 1.8 | 0.6 | 1.9 |
| (=G2F + NANA) | | | | | | | | | |
| Glycan form - % G2FS1 | 27 | ND | 0.9 | Not detected | | | | | |
| (=G2F + 2NANA) | | | | | | | | | |

*Traces: <0.2%

TABLE 30
Reference Standard - Glycan Forms

| | | | | | | Lower | Upper | Lower | Upper |
|---|---|---|---|---|---|---|---|---|---|
| Parameter | N | Min | Max | Mean | Std Dev | 95% TI | 95% TI | 99% TI | 99% TI |
| Glycan form - % G0-GlcNAc | Not detected (ND) - Traces* | | | | | | | | |
| Glycan form - % G0F-GlcNAc | 5 | Traces* | 0.4 | Not applicable | | | | | |
| Glycan form - % G0 | 10 | 2.5 | 3.2 | 2.9 | 0.210 | 1.9 | 3.8 | 1.7 | 4.1 |
| Glycan form - % G1-GlcNAc | 4 | ND | 0.8 | Not applicable | | | | | |
| Glycan form - % G0F | 10 | 53.8 | 63.1 | 56.8 | 2.751 | 44.3 | 68.7 | 41.0 | 71.9 |
| Glycan form - % Man5 | 10 | 0.7 | 1.1 | 0.9 | 0.133 | 0.3 | 1.5 | 0.2 | 1.6 |
| Glycan form - % G1F-GlcNAc/G1a | 8 | ND | 0.6 | Not applicable | | | | | |
| Glycan form - % G1a | 2 | ND | 0.5 | Not applicable | | | | | |
| Glycan form - % G1b | 8 | Traces* | 0.4 | Not applicable | | | | | |
| Glycan form - % G0FB | 7 | ND | 0.8 | Not applicable | | | | | |
| Glycan form - % G1Fa | 10 | 17.3 | 20.4 | 19.6 | 0.858 | 15.7 | 23.4 | 14.7 | 24.4 |
| Glycan form - % G1Fb | 10 | 6.2 | 7.6 | 7.1 | 0.414 | 5.3 | 9.0 | 4.8 | 9.5 |
| Glycan form - % Man6 | Not detected (ND) - Traces* | | | | | | | | |
| Glycan form - % G2 | 4 | ND | 0.6 | Not applicable | | | | | |
| Glycan form - % G2/G1F + NANA | 2 | ND | 0.5 | Not applicable | | | | | |
| Glycan form - % G1FB | 6 | ND | 0.9 | Not applicable | | | | | |
| Glycan form - % G2F | 10 | 6.2 | 9.2 | 8.4 | 0.800 | 4.8 | 11.9 | 3.9 | 12.8 |
| Glycan form - % G2FB | Not detected (ND) - Traces* | | | | | | | | |
| Glycan form - % G2F + Gal | Not detected (ND) - Traces* | | | | | | | | |
| Glycan form - % G2FS1 | 10 | 0.9 | 1.6 | 1.4 | 0.217 | 0.4 | 2.3 | 0.1 | 2.6 |
| (=G2F + NANA) | | | | | | | | | |
| Glycan form - % G2FS1 | 6 | ND | 0.9 | Not detected | | | | | |
| (=G2F + 2NANA) | | | | | | | | | |

*Traces: <0.2%

TABLE 31
Drug Substance & Reference Standard - Glycan Forms (November 2020)

| | | | | | | G1F- | | | | | | |
|---|---|---|---|---|---|---|---|---|---|---|---|---|
| | G0F- | | G1- | | | GlcNAc/ | | | | | | G2F + |
| Batch | GlcNAc | G0 | GlcNAc | G0F | Man5 | G1a | G1b | G1Fa | G1Fb | G2 | G2F | NANA |
| Batch A | 0.4 | 3.5 | 0.4 | 64.1 | 1.0 | 0.5 | 0.4 | 16.7 | 6.1 | 0.5 | 5.6 | 0.8 |
| Batch B | 0.3 | 2.8 | 0.5 | 60.6 | 0.9 | 0.5 | 0.3 | 18.5 | 6.5 | 0.5 | 7.5 | 1.0 |
| Batch C | 0.6 | 3.0 | 0.9 | 58.1 | 1.0 | 0.6 | 0.4 | 19.3 | 6.5 | 0.6 | 8.0 | 1.0 |
| Batch D | 0.4 | 2.8 | 0.7 | 59.2 | 0.8 | 0.5 | 0.4 | 19.0 | 6.7 | 0.5 | 7.8 | 1.2 |
| Batch E | 0.6 | 3.1 | 1.6 | 57.0 | 0.8 | 0.6 | 0.4 | 19.9 | 6.6 | 0.6 | 7.9 | 0.9 |
| Batch F | 0.4 | 2.8 | 0.6 | 56.6 | 0.9 | 0.6 | 0.4 | 20.7 | 7.0 | 0.5 | 8.6 | 1.1 |
| Batch G | 0.4 | 2.7 | 1.4 | 56.8 | 0.7 | 0.6 | 0.4 | 20.1 | 6.8 | 0.6 | 8.4 | 1.2 |
| Batch H | 0.4 | 3.2 | 0.6 | 63.1 | 0.7 | 0.5 | 0.3 | 17.3 | 6.2 | 0.5 | 6.2 | 0.9 |
| Batch 2 | 0.4 | 2.7 | 0.8 | 57.1 | 0.8 | 0.6 | 0.4 | 20.0 | 6.9 | 0.6 | 8.4 | 1.2 |
| Batch 1 | 0.4 | 2.7 | 0.7 | 57.4 | 0.8 | 0.6 | 0.4 | 19.9 | 6.9 | 0.6 | 8.4 | 1.2 |

TABLE 32
Drug Substance & Reference Standard - Glycan Forms (June 2021)

| | | | | | | G1F- | | | | | | | |
|---|---|---|---|---|---|---|---|---|---|---|---|---|---|
| | G0F- | | G1- | | | GlcNAc/ | | | | | | | G2F + |
| Batch | GlcNAc | G0 | GlcNAc | G0F | Man5 | G1a | G1b | G0FB | G1Fa | G1Fb | G2 | G2F | NANA |
| Batch D | 0.3 | 3.3 | 0.4 | 61.5 | 1.1 | 0.4 | 0.4 | 0.8 | 17.1 | 6.4 | 0.5 | 6.7 | 1.0 |
| Batch E | 0.3 | 3.0 | 0.4 | 60.1 | 1.1 | 0.4 | 0.4 | 0.8 | 17.8 | 6.5 | 0.5 | 7.4 | 1.2 |
| Batch F | 0.3 | 3.7 | 0.4 | 59.5 | 1.2 | 0.5 | 0.4 | 0.8 | 17.8 | 6.5 | 0.5 | 7.4 | 1.1 |
| Batch K | 0.4 | 2.6 | 0.9 | 59.2 | 1.2 | 0.4 | 0.4 | 0.9 | 18.0 | 6.6 | 0.5 | 7.8 | 1.1 |
| Batch L | 0.4 | 2.8 | 0.8 | 57.7 | 1.2 | 0.5 | 0.4 | 0.9 | 18.8 | 6.8 | 0.5 | 8.1 | 1.1 |
| Batch M | 0.3 | 2.5 | 0.3 | 56.4 | 1.1 | 0.4 | 0.3 | 0.7 | 20.0 | 7.2 | 0.5 | 8.9 | 1.2 |
| Batch 2 | 0.3 | 2.5 | 0.4 | 57.8 | 1.1 | 0.4 | 0.3 | 0.7 | 19.2 | 7.1 | 0.5 | 8.5 | 1.2 |

TABLE 33
Drug Substance & Reference Standard - Glycan Forms (February 2022)

| | | | | | | G1F- | | | | | | | | | |
|---|---|---|---|---|---|---|---|---|---|---|---|---|---|---|---|
| | G0F- | | G1- | | | GlcNAc/ | | | | | | | | G2F + | G2F + |
| Batch | GlcNAc | G0 | GlcNAc | G0F | Man5 | G1a | G1b | G0FB | G1Fa | G1Fb | G2 | G1FB | G2F | NANA | 2NANA |
| Batch 2 | Traces | 2.9 | ND | 56.3 | 1.0 | 0.3 | Traces | 0.6 | 19.7 | 7.1 | ND | 0.8 | 8.6 | 1.4 | 0.8 |
| Batch N | Traces | 3.7 | ND | 57.4 | 1.4 | 0.3 | Traces | 0.9 | 17.8 | 7.2 | ND | 1.0 | 7.5 | 1.3 | 0.7 |
| Batch O | Traces | 4.2 | ND | 59.7 | 1.2 | 0.4 | 0.3 | 0.8 | 16.5 | 6.5 | ND | 0.8 | 6.9 | 1.3 | 0.9 |
| Batch P | Traces | 2.9 | ND | 55.5 | 1.1 | 0.4 | Traces | 0.7 | 19.7 | 7.4 | ND | 0.8 | 8.3 | 1.5 | 0.9 |
| Batch Q | Traces | 4.5 | ND | 58.1 | 1.1 | 0.4 | 0.3 | 0.6 | 17.8 | 6.6 | ND | 0.8 | 7.1 | 1.3 | 0.7 |
| Batch R | Traces | 3.8 | ND | 57.3 | 1.1 | 0.4 | 0.3 | 0.6 | 18.4 | 6.9 | ND | 0.7 | 7.7 | 1.4 | 0.7 |
| Batch S | Traces | 3.6 | ND | 58.3 | 1.0 | 0.3 | Traces | 0.6 | 18.1 | 6.7 | ND | 0.7 | 7.5 | 1.4 | 0.8 |
| Batch T | Traces | 3.5 | ND | 59.8 | 1.0 | 0.3 | Traces | 0.6 | 17.6 | 6.8 | ND | 0.7 | 6.9 | 1.2 | 0.7 |
| Batch U | Traces | 3.5 | ND | 58.5 | 1.1 | 0.3 | Traces | 0.6 | 18.2 | 6.7 | ND | 0.7 | 7.5 | 1.4 | 0.8 |

Traces: <0.2;
ND: not detected

TABLE 34
Drug Substance & Reference Standard - Glycan Forms (March 2022)

| | | | | | | G1F- | | | | | | | | | | |
|---|---|---|---|---|---|---|---|---|---|---|---|---|---|---|---|---|
| | G0F- | | G1- | | | GlcNAc/ | | | | | | | | | G2F + | G2F + |
| Batch | GlcNAc | G0 | GlcNAc | G0F | Man5 | G1a | G1a | G1b | G0FB | G1Fa | G1Fb | G2 | G1FB | G2F | NANA | 2NANA |
| Batch 1 | Traces | 3.0 | ND | 53.8 | 0.9 | ND | 0.5 | 0.4 | 0.7 | 19.9 | 7.5 | ND | 0.8 | 9.2 | 1.5 | 0.8 |
| Batch P | 0.3 | 3.1 | ND | 53.9 | 0.9 | ND | 0.5 | 0.4 | 0.8 | 20.0 | 7.5 | ND | 0.9 | 8.6 | 1.5 | 0.9 |
| Batch V | 0.3 | 3.4 | ND | 59.5 | 0.9 | ND | 0.4 | 0.4 | 0.9 | 17.5 | 6.7 | ND | 0.9 | 6.7 | 1.2 | 0.7 |
| Batch W | 0.3 | 3.4 | ND | 57.3 | 1.0 | ND | 0.4 | 0.4 | 0.8 | 18.5 | 6.9 | ND | 0.8 | 7.5 | 1.2 | 0.7 |
| Batch X | 0.3 | 4.5 | ND | 58.9 | 1.2 | ND | 0.5 | 0.4 | 0.8 | 17.2 | 6.5 | ND | 0.8 | 6.7 | 1.1 | 0.6 |
| Batch Y | Traces | 3.6 | ND | 55.2 | 1.0 | ND | 0.5 | 0.4 | 0.8 | 19.1 | 7.3 | ND | 0.8 | 8.2 | 1.4 | 0.8 |
| Batch Z | 0.3 | 3.4 | ND | 57.4 | 1.1 | ND | 0.4 | 0.4 | 0.8 | 18.1 | 6.9 | ND | 0.8 | 7.5 | 1.3 | 0.8 |
| Batch | Traces | 3.1 | ND | 54.7 | 1.0 | ND | 0.5 | 0.4 | 0.7 | 19.7 | 7.2 | ND | 0.8 | 8.6 | 1.5 | 0.9 |
| AA | | | | | | | | | | | | | | | | |
| Batch Q | 0.3 | 4.5 | ND | 56.0 | 1.0 | ND | 0.5 | 0.5 | 0.8 | 18.2 | 6.9 | ND | 0.8 | 7.6 | 1.3 | 0.7 |
| Batch I | 0.3 | 3.5 | ND | 58.3 | 1.1 | ND | 0.4 | 0.4 | 0.9 | 17.3 | 6.6 | ND | 0.9 | 7.4 | 1.4 | 0.8 |

Traces: <0.2;
ND: not detected

TABLE 35
Drug Substance & Reference Standard - Glycan Forms (August 2022)

| | | | | | | | | G1F- | | |
|---|---|---|---|---|---|---|---|---|---|---|
| | Man. | | G0F- | | G1- | | | GlcNAc/ | | |
| Batch | Site | Formulation | GlcNAc | G0 | GlcNAc | G0F | Man5 | G1a | G1a | G1b |
| Batch 2 | Slough, | IV-20 | Traces | 3.0 | ND | 55.9 | 0.9 | 0.4 | ND | 0.4 |
| | UK | | | | | | | | | |
| Batch P | Tuas, | SC-180 | Traces | 2.8 | ND | 54.0 | 0.8 | 0.4 | ND | 0.4 |
| | SNG | | | | | | | | | |
| Batch K | Tuas, | SC-180 | Traces | 3.8 | ND | 59.6 | 0.9 | 0.4 | ND | 0.4 |
| | SNG | | | | | | | | | |
| Batch L | Tuas, | SC-180 | Traces | 3.7 | ND | 58.9 | 0.9 | 0.4 | ND | 0.4 |
| | SNG | | | | | | | | | |
| Batch M | Tuas, | SC-180 | Traces | 4.2 | ND | 57.3 | 0.9 | 0.4 | ND | 0.4 |
| | SNG | | | | | | | | | |
| Batch Q | Tuas, | SC-180 | Traces | 4.1 | ND | 56.3 | 0.9 | 0.5 | ND | 0.5 |
| | SNG | | | | | | | | | |
| Batch R | Tuas, | SC-180 | Traces | 3.5 | ND | 55.4 | 0.9 | 0.4 | ND | 0.4 |
| | SNG | | | | | | | | | |
| Batch J | Tuas, | SC-180 | Traces | 4.2 | ND | 59.2 | 0.9 | 0.4 | ND | 0.4 |
| | SNG | | | | | | | | | |

| | | | | | G2/ | | | | |
|---|---|---|---|---|---|---|---|---|---|
| | | | | | G1F + | | | G2F + | G2F + |
| Batch | G0FB | G1Fa | G1Fb | G2 | NANA | G1FB | G2F | NANA | 2NANA |
| Batch 2 | 0.7 | 19.4 | 7.1 | ND | 0.4 | 0.5 | 8.6 | 1.5 | 0.8 |
| Batch P | 0.8 | 20.4 | 7.6 | ND | 0.5 | 0.5 | 8.8 | 1.6 | 0.9 |
| Batch K | 0.8 | 17.4 | 6.5 | ND | 0.4 | 0.5 | 6.7 | 1.3 | 0.7 |
| Batch L | 0.7 | 17.9 | 6.5 | ND | 0.4 | 0.4 | 7.2 | 1.3 | 0.7 |
| Batch M | 0.8 | 18.2 | 6.6 | ND | 0.4 | 0.5 | 7.5 | 1.4 | 0.8 |
| Batch Q | 0.8 | 18.5 | 7.0 | ND | 0.4 | 0.5 | 7.6 | 1.4 | 0.8 |
| Batch R | 0.8 | 19.2 | 7.2 | ND | 0.4 | 0.5 | 8.4 | 1.5 | 0.9 |
| Batch J | 0.9 | 17.2 | 6.6 | ND | 0.4 | 0.5 | 6.5 | 1.3 | 0.8 |

Traces: <0.2;
ND: not detected

The invention is not to be limited in scope by the specific embodiments described herein. Indeed, various modifications of the invention in addition to those described will become apparent to those skilled in the art from the foregoing description and accompanying figures. Such modifications are intended to fall within the scope of the appended claims.

1. A composition comprising a population of FcRn antagonist molecules, wherein at least a portion of the FcRn antagonist molecules in the population consist of a variant Fc region comprising a dimer of a first Fc domain and a second Fc domain, wherein the amino acid sequence of the first and second Fc domain consists of SEQ ID NO: 1, provided that the population is not a homogeneous population of homodimeric FcRn antagonist molecules in which the amino acid sequence of both the first and the second Fc domain consists of SEQ ID NO: 2, 3, 20, or 21.

2. The composition of claim 1, wherein each FcRn antagonist molecule in the population consists of a variant Fc region comprising a dimer of a first Fc domain and a second Fc domain, wherein the amino acid sequence of the first and second Fc domain consists of SEQ ID NO: 1.

3. The composition of claim 1, wherein the amino acid sequences of the first and the second Fc domains consist of SEQ ID NO: 3 and SEQ ID NO: 12, respectively.

4. The composition of claim 1, wherein the amino acid sequences of the first and the second Fc domains consist of SEQ ID NO: 3 and SEQ ID NO: 9, respectively.

5. The composition of claim 1, wherein the amino acid sequences of the first and the second Fc domains consist of SEQ ID NO: 2 and SEQ ID NO: 3, respectively.

6. The composition of claim 1, wherein the amino acid sequences of the first and the second Fc domains consist of SEQ ID NO: 3 and SEQ ID NO: 6, respectively.

7. The composition of claim 1, wherein the amino acid sequence of the first Fc domain consists of any one of SEQ ID NOs: 2-22, and the amino acid sequence of the second Fc domain consists of any one of SEQ ID NOs: 2-22.

8. The composition of claim 1, wherein the amino acid sequence of both the first and the second Fc domain consists of any one of SEQ ID NOs: 5-19 or 22.

9.-23. (canceled)

24. The composition of claim 1, wherein the population comprises:
(a) a first subpopulation of FcRn antagonist molecules, wherein the amino acid sequences of both the first and the second Fc domains of the FcRn antagonist molecules in the first subpopulation consist of SEQ ID NO: 3; and
(b) at least one of:
(i) a second subpopulation of FcRn antagonist molecules, wherein the amino acid sequences of the first and the second Fc domains of the FcRn antagonist molecules in the second subpopulation consist of SEQ ID NO: 3 and SEQ ID NO: 12, respectively;
(ii) a third subpopulation of FcRn antagonist molecules, wherein the amino acid sequences of the first and the second Fc domains of the FcRn antagonist molecules in the third subpopulation consist of SEQ ID NO: 3 and SEQ ID NO: 9, respectively;
(iii) a fourth subpopulation of FcRn antagonist molecules, wherein the amino acid sequences of both the first and the second Fc domains of the FcRn antagonist molecules in the fourth subpopulation consist of SEQ ID NO: 3, and wherein two asparagine residues in each FcRn antagonist molecule in the fourth subpopulation are deaminated;
(iv) a fifth subpopulation of FcRn antagonist molecules, wherein the amino acid sequences of both the first and the second Fc domains of the FcRn antagonist molecules in the fifth subpopulation consist of SEQ ID NO: 3, and wherein one asparagine residue in each FcRn antagonist molecule in the fifth subpopulation is deaminated;
(v) a sixth subpopulation of FcRn antagonist molecules, wherein the amino acid sequences of the first and the second Fc domains of the FcRn antagonist molecules in the sixth subpopulation consist of SEQ ID NO: 2 and SEQ ID NO: 3, respectively;
(vi) a seventh subpopulation of FcRn antagonist molecules, wherein the amino acid sequences of both the first and the second Fc domains of the FcRn antagonist molecules in the seventh subpopulation consist of SEQ ID NO: 3, and wherein one methionine residue or one tryptophan residue in each FcRn antagonist molecule in the seventh subpopulation is oxidized;
(vii) an eighth subpopulation of FcRn antagonist molecules, wherein the amino acid sequences of both the first and the second Fc domains of the FcRn antagonist molecules in the eighth subpopulation consist of SEQ ID NO: 2;
(viii) a ninth subpopulation of FcRn antagonist molecules, wherein the amino acid sequences of the first and the second Fc domains of the FcRn antagonist molecules in the ninth subpopulation consist of SEQ ID NO: 3 and SEQ ID NO: 6, respectively;
(ix) a tenth subpopulation of FcRn antagonist molecules, wherein the amino acid sequences of the first and the second Fc domains of the FcRn antagonist molecules in the tenth subpopulation consist of SEQ ID NO: 2 and SEQ ID NO: 3, respectively, and wherein one methionine residue or one tryptophan residue in each FcRn antagonist molecule in the tenth subpopulation is oxidized; and
(x) an eleventh subpopulation of FcRn antagonist molecules, wherein the amino acid sequences of both the first and the second Fc domains of the FcRn antagonist molecules in the eleventh subpopulation consist of SEQ ID NO: 3, and wherein two amino acid residues, independently selected from a methionine residue and a tryptophan residue, in each FcRn antagonist molecule in the eleventh subpopulation are oxidized.

25. The composition of claim 24, wherein the population comprises 2, 3, 4, 5, 6, 7, 8, 9, 10, or all of the subpopulations set forth in (b).

26.-27. (canceled)

28. The composition of claim 24, wherein at least one of:
(a) the first subpopulation is at least 55% of the population;
(b) the second subpopulation is no more than 2.5% of the population;
(c) the third subpopulation is no more than 2.5% of the population;
(d) the fourth subpopulation is no more than 4% of the population;
(e) the fifth subpopulation is no more than 10% of the population;
(f) the sixth subpopulation is no more than 20% of the population;
(g) the seventh subpopulation is no more than 6% of the population;
(h) the eighth subpopulation is no more than 8% of the population;
(i) the ninth subpopulation is no more than 3.5% of the population;
(j) the tenth subpopulation is no more than 1% of the population; and
(k) the eleventh subpopulation is no more than 1% of the population.

29.-38. (canceled)

39. The composition of claim 1, wherein;
(a) at least 97% of the Fc domains in the population comprise an N-glycan at EU position 297;
(b) at least 50% of the Fc domains in the population comprise a G0F N-glycan at EU position 297;
(c) at least 20% of the Fc domains in the population comprise a G1F N-glycan at EU position 297;
(d) at least 5% of the Fc domains in the population comprise a G2F N-glycan at EU position 297;
(e) at least 2% of the Fc domains in the population comprise a G0 N-glycan at EU position 297;
(f) at least 40% of the population comprise a first Fc domain comprising a G0F N-glycan at EU position 297 and a second Fc domain comprising a G0F N-glycan at EU position 297;
(g) at least 20% of the population comprise a first Fc domain comprising a G0F N-glycan at EU position 297 and a second Fc domain comprising a G1F N-glycan at EU position 297;
(h) at least 10% of the population comprise either a first Fc domain comprising a G1F N-glycan at EU position 297 and a second Fc domain comprising a G1F N-glycan at EU position 297, or a first Fc domain comprising G0F N-glycan at EU position 297 and a second Fc domain comprising a G2F N-glycan at EU position 297;
(i) at least 5% of the population comprise a first Fc domain comprising a G1F N-glycan at EU position 297 and a second Fc domain comprising a G2F N-glycan at EU position 297;
(j) at least 2% of the population comprise a first Fc domain comprising a G2F N-glycan at EU position 297 and a second Fc domain comprising a G2F N-glycan at EU position 297; or
(k) at least 4% of the population comprise a first Fc domain comprising a G0F N-glycan at EU position 297 and a second Fc domain comprising a G0 N-glycan at EU position 297.

40.-49. (canceled)

50. A composition comprising an FcRn antagonist molecule consisting of a variant Fc region comprising a dimer of a first Fc domain and a second Fc domain, wherein the amino acid sequence of the first and second Fc domain consists of SEQ ID NO: 1, and wherein at least one Fc domain comprises a G0F N-glycan at EU position 297, a G1F N-glycan at EU position 297, a G2F N-glycan at EU position 297, or a G0 N-glycan at EU position 297.

51. The composition of claim 50, wherein:
(a) the first Fc domain comprises a G0F N-glycan at EU position 297, and the second Fc domain comprises a G0F N-glycan at EU position 297, a G1F N-glycan at EU position 297, or a G2F N-glycan at EU position 297;
(b) the first Fc domain comprises a G1F N-glycan at EU position 297, and the second Fc domain comprises a G1F N-glycan at EU position 297;
(c) the first Fc domain comprises a G2F N-glycan at EU position 297, and the second Fc domain comprises a G2F N-glycan at EU position 297;
(d) the first Fc domain comprises a G0F N-glycan at EU position 297, and the second Fc domain comprises a G0 N-glycan at EU position 297;
(e) the first Fc domain comprises a G0 N-glycan at EU position 297, and the second Fc domain comprises a G0 N-glycan at EU position 297;
(f) the first Fc domain comprises a G1F N-glycan at EU position 297, and the second Fc domain comprises a G2F+NANA N-glycan at EU position 297; or
(g) the first Fc domain comprises a G2F N-glycan at EU position 297, and the second Fc domain comprises a G2F+2×NANA N-glycan at EU position 297.

52.-59. (canceled)

60. The composition of claim 50, wherein;
(a) the amino acid sequences of the first and the second Fc domains consist of SEQ ID NO: 3 and SEQ ID NO: 12, respectively;
(b) the amino acid sequences of the first and the second Fc domains consist of SEQ ID NO: 3 and SEQ ID NO: 9, respectively;
(c) the amino acid sequences of the first and the second Fc domains consist of SEQ ID NO: 2 and SEQ ID NO: 3, respectively;
(d) the amino acid sequences of the first and the second Fc domains consist of SEQ ID NO: 3 and SEQ ID NO: 6, respectively;
(e) the amino acid sequence of the first Fc domain consists of any one of SEQ ID NOs: 2-22, and the amino acid sequence of the second Fc domain consists of any one of SEQ ID NOs: 2-22;
(f) the amino acid sequence of both the first and the second Fc domain consists of SEQ ID NO: 2;
(g) the amino acid sequence of both the first and the second Fc domain consists of SEQ ID NO: 3;
(h) the amino acid sequence of both the first and the second Fc domain consists of SEQ ID NO: 4;
(i) the amino acid sequence of both the first and the second Fc domain consists of SEQ ID NO: 5;
(j) the amino acid sequence of both the first and the second Fc domain consists of SEQ ID NO: 6;
(k) the amino acid sequence of both the first and the second Fc domain consists of SEQ ID NO: 7;
(l) the amino acid sequence of both the first and the second Fc domain consists of SEQ ID NO: 8;
(m) the amino acid sequence of both the first and the second Fc domain consists of SEQ ID NO: 9;
(n) the amino acid sequence of both the first and the second Fc domain consists of SEQ ID NO: 10;
(o) the amino acid sequence of both the first and the second Fc domain consists of SEQ ID NO: 11;
(p) the amino acid sequence of both the first and the second Fc domain consists of SEQ ID NO: 12;
(g) the amino acid sequence of both the first and the second Fc domain consists of SEQ ID NO: 13;
(r) the amino acid sequence of both the first and the second Fc domain consists of SEQ ID NO: 14;
(s) the amino acid sequence of both the first and the second Fc domain consists of SEQ ID NO: 15;
(t) the amino acid sequence of both the first and the second Fc domain consists of SEQ ID NO: 16;
(u) the amino acid sequence of both the first and the second Fc domain consists of SEQ ID NO: 17;
(v) the amino acid sequence of both the first and the second Fc domain consists of SEQ ID NO: 18;
(w) the amino acid sequence of both the first and the second Fc domain consists of SEQ ID NO: 19;
(x) the amino acid sequence of both the first and the second Fc domain consists of SEQ ID NO: 20;
(y) the amino acid sequence of both the first and the second Fc domain consists of SEQ ID NO: 21; or
(z) the amino acid sequence of both the first and the second Fc domain consists of SEQ ID NO: 22.

61.-85. (canceled)

86. The composition of claim 1, wherein at least 85% of the Fc domains in the population lack an amino acid at EU position 441.

87.-88. (canceled)

89. The composition of claim 1, wherein:
(a) at least 95%, optionally 95% to 99%, of the Fc domains in the population have aspartate, lysine, threonine, histidine, threonine, and cysteine, at EU positions 221, 222, 223, 224, 225, and 226, respectively;
(b) no more than 1% of the Fc domains in the population lack an amino acid at EU position 221, and have lysine, threonine, histidine, threonine, and cysteine at EU positions 222, 223, 224, 225, and 226, respectively;
(c) no more than 1% of the Fc domains in the population lack amino acids at EU positions 221 and 222, and have threonine, histidine, threonine, and cysteine at EU positions 223, 224, 225, and 226, respectively;
(d) no more than 2% of the Fc domains in the population lack amino acids at EU positions 221-224, and have threonine, and cysteine at EU positions 225 and 226, respectively; and/or
(e) no more than 1% of the Fc domains in the population lack amino acids at EU positions 221, 222, 223, 224, 225, and 226.

90.-93. (canceled)

94. The composition of claim 1, wherein;
(a) no more than 1% of the Fc domains in the population have isomerization of the aspartate at EU position 280 or 401;
(b) no more than 10% of the Fc domains in the population have deamidation of the asparagine at EU position 384, 389, or 390;
(c) no more than 3% of the Fc domains in the population have deamidation of the asparagine at EU position 315;
(d) no more than 3% of the Fc domains in the population have deamidation of the asparagine at EU position 361;
(e) no more than 1% of the Fc domains in the population have deamidation of the asparagine at EU position 276 or 286;
(f) no more than 5% of the Fc domains have oxidization of the methionine at EU position 428;
(g) no more than 1% of the Fc domains have amidation of the proline at EU position 445; and/or
(h) no more than 1% of the Fc domains have oxidization of the tryptophan at EU position 277.

95.-101. (canceled)

102. The composition of claim 1, wherein no more than 0.5% of the FcRn antagonist molecules in the population are aggregated.

103. The composition of claim 1, wherein at least 95% of the dimers in the population are linked by at least one disulfide bond.

104. The composition of claim 1, wherein the average molecular weight of non-aggregated FcRn antagonist molecules in the population is 54 to 56 kDa.

105. The composition of claim 1, wherein the percentage of free thiol groups in the population is no more than 1%.

106. The composition of claim 1, wherein:
(a) at least 35% of the Fc domains in the population comprise galactose;
(b) at least 90% of the Fc domains in the population comprise fucose; and/or
(c) at most 1.5% of the Fc domains in the population comprise sialic acid.

107.-108. (canceled)

109. The composition of claim 1, which is an aqueous solution comprising about 25 mM sodium phosphate, about 100 mM sodium chloride, and about 150 mM L-arginine, and about 0.02% (w/v) polysorbate 80, wherein the composition has a pH of about 6.7.

110. The composition of claim 109, comprising 20 mg/ml of the population of FcRn antagonist molecules.

111.-112. (canceled)

113. The composition of claim 1, which is an aqueous solution comprising about 20 mM L-histidine, about 100 mM sodium chloride, about 60 mM sucrose, about 10 mM L-methionine, and about 0.04% (w/v) polysorbate 20, wherein the composition has a pH of about 6.0.

114. The composition of claim 113, comprising about 180 mg/ml of the population of FcRn antagonist molecules.

115.-116. (canceled)

117. An FcRn antagonist molecule consisting of a variant Fc region comprising a homodimer of a first Fc domain and a second Fc domain, wherein the amino acid sequence of both the first and the second Fc domain consists of any one of SEQ ID NOs: 5-20 and 22.

118. A polynucleotide encoding the FcRn antagonist molecule of claim 117.

119. A vector comprising the polynucleotide of claim 118.

120. A cell comprising the polynucleotide of claim 118.

121. A method of making an FcRn antagonist molecule, the method comprising culturing the cell of claim 120 under conditions such that the polynucleotide is expressed and the FcRn antagonist molecule is produced.

122. The method of claim 121, further comprising isolating the FcRn antagonist molecule from the cell.

123. A method comprising mixing the composition of claim 1 with one or more pharmaceutically acceptable excipients.

124. A method of reducing the level of serum IgG autoantibodies in a subject, the method comprising administering to the subject the composition of claim 1.

125. A method of treating an autoimmune disease in a subject, the method comprising administering to the subject the composition of claim 1.

126.-129. (canceled)


SEQUENCE LISTING (36 sequences)

SEQ ID NO: 1 - protein, 227 aa, synthetic construct
  VARIANT 1: note = D or absent
  VARIANT 2: note = K or absent
  VARIANT 3: note = T or absent
  VARIANT 4: note = H or absent
  VARIANT 5: note = T or absent
  VARIANT 6: note = C or absent
  VARIANT 226: note = G or absent
  VARIANT 227: note = K or absent
XXXXXXPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPXX

SEQ ID NO: 2 - protein, 227 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 3 - protein, 226 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 4 - protein, 225 aa, synthetic construct
DKTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 5 - protein, 226 aa, synthetic construct
KTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 6 - protein, 225 aa, synthetic construct
KTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 7 - protein, 224 aa, synthetic construct
KTHTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 8 - protein, 225 aa, synthetic construct
THTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 9 - protein, 224 aa, synthetic construct
THTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 10 - protein, 223 aa, synthetic construct
THTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 11 - protein, 223 aa, synthetic construct
TCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 12 - protein, 222 aa, synthetic construct
TCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 13 - protein, 221 aa, synthetic construct
TCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 14 - protein, 221 aa, synthetic construct
PPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 15 - protein, 220 aa, synthetic construct
PPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 16 - protein, 219 aa, synthetic construct
PPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 17 - protein, 224 aa, synthetic construct
HTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 18 - protein, 223 aa, synthetic construct
HTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 19 - protein, 222 aa, synthetic construct
HTCPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 20 - protein, 221 aa, synthetic construct
CPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPG

SEQ ID NO: 21 - protein, 222 aa, synthetic construct
CPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSPGK

SEQ ID NO: 22 - protein, 220 aa, synthetic construct
CPPCPAPELLGGPSVFLFPPKPKDTLYITREPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALKFHYTQKSLSLSP

SEQ ID NO: 23 - protein, 15 aa, synthetic construct
EPKSCDKTHTCPPCP

SEQ ID NO: 24 - protein, 22 aa, synthetic construct
GFYPSDIAVEWESNGQPENNYK

SEQ ID NO: 25 - protein, 16 aa, synthetic construct
VVSVLTVLHQDWLNGK

SEQ ID NO: 26 - protein, 10 aa, synthetic construct
NQVSLTCLVK

SEQ ID NO: 27 - protein, 24 aa, synthetic construct
TCPPCPAPELLGGPSVFLFPPKPK

SEQ ID NO: 28 - protein, 28 aa, synthetic construct
DKTHTCPPCPAPELLGGPSVFLFPPKPK

SEQ ID NO: 29 - protein, 26 aa, synthetic construct
THTCPPCPAPELLGGPSVFLFPPKPK

SEQ ID NO: 30 - protein, 8 aa, synthetic construct
SLSLSPGK

SEQ ID NO: 31 - protein, 6 aa, synthetic construct
SLSLSP

SEQ ID NO: 32 - protein, 7 aa, synthetic construct
SLSLSPG

SEQ ID NO: 33 - protein, 17 aa, synthetic construct
TTPPVLDSDGSFFLYSK

SEQ ID NO: 34 - protein, 14 aa, synthetic construct
FNWYVDGVEVHNAK

SEQ ID NO: 35 - protein, 17 aa, synthetic construct
WQQGNVFSCSVMHEALK

SEQ ID NO: 36 - protein, 9 aa, synthetic construct
EEQYNSTYR